SEQUENCE LISTING (71 sequences)

SEQ ID NO: 1 - protein, 107 aa, Homo sapiens
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 2 - DNA, 324 bp, Homo sapiens
atgggagtgcaggtggagactattagccccggagatggcagaacattccccaaaagaggacagacttgcgtcgtgcattatactggaatgctggaagacggcaagaaggtggacagcagccgggaccgaaacaagcccttcaagttcatgctggggaagcaggaagtgatccggggctgggaggaaggagtcgcacagatgtcagtgggacagagggccaaactgactattagcccagactacgcttatggagcaaccggccaccccgggatcattccccctcatgctacactggtcttcgatgtggagctgctgaagctggaa

SEQ ID NO: 3 - protein, 8 aa, Artificial Sequence
  Description of Artificial Sequence: Synthetic peptide
SGGGSGVD

SEQ ID NO: 4 - DNA, 24 bp, Artificial Sequence
  Description of Artificial Sequence: Synthetic oligonucleotide
agcggaggaggatccggagtggac

SEQ ID NO: 5 - protein, 18 aa, Thosea asigna virus
EGRGSLLTCGDVEENPGP

SEQ ID NO: 6 - DNA, 54 bp, Thosea asigna virus
gaaggccgagggagcctgctgacatgtggcgatgtggaggaaaacccaggacca

SEQ ID NO: 7 - protein, 21 aa, Artificial Sequence
  Description of Artificial Sequence: Synthetic peptide
MEFGLSWLFLVAILKGVQCSR

SEQ ID NO: 8 - DNA, 63 bp, Artificial Sequence
  Description of Artificial Sequence: Synthetic oligonucleotide
atggagtttggactttcttggttgtttttggtggcaattctgaagggtgtccagtgtagcagg

SEQ ID NO: 9 - protein, 8 aa, Artificial Sequence
  Description of Artificial Sequence: Synthetic peptide
GGGSGGGG

SEQ ID NO: 10 - DNA, 24 bp, Artificial Sequence
  Description of Artificial Sequence: Synthetic oligonucleotide
ggcggaggaagcggaggtgggggc

SEQ ID NO: 11 - protein, 16 aa, Homo sapiens
ELPTQGTFSNVSTNVS

SEQ ID NO: 12 - DNA, 48 bp, Homo sapiens
gaacttcctactcaggggactttctcaaacgttagcacaaacgtaagt

SEQ ID NO: 13 - protein, 42 aa, Homo sapiens
PAPRPPTPAPTIASQPLSLRPEACRPAAGGAVHTRGLDFACD

SEQ ID NO: 14 - DNA, 126 bp, Homo sapiens
cccgccccaagaccccccacacctgcgccgaccattgcttctcaacccctgagtttgagacccgaggcctgccggccagctgccggcggggccgtgcatacaagaggactcgatttcgcttgcgac

SEQ ID NO: 15 - protein, 37 aa, Homo sapiens
IYIWAPLAGTCGVLLLSLVITLYCNHRNRRRVCKCPR

SEQ ID NO: 16 - DNA, 111 bp, Homo sapiens
atctatatctgggcacctctcgctggcacctgtggagtccttctgctcagcctggttattactctgtactgtaatcaccggaatcgccgccgcgtttgtaagtgtcccagg

SEQ ID NO: 17 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - DNA, 336 bp, Homo sapiens
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaagctcttccacctcgt

SEQ ID NO: 19 - protein, 19 aa, Porcine teschovirus 1
ATNFSLLKQAGDVEENPGP

SEQ ID NO: 20 - DNA, 57 bp, Porcine teschovirus 1
gcaacgaatttttccctgctgaaacaggcaggggacgtagaggaaaatcctggtcct

SEQ ID NO: 21 - protein, 172 aa, Homo sapiens
MAAGGPGAGSAAPVSSTSSLPLAALNMRVRRRLSLFLNVRTQVAADWTALAEEMDFEYLEIRQLETQADPTGRLLDAWQGRPGASVGRLLDLLTKLGRDDVLLELGPSIEEDCQKYILKQQQEEAEKPLQVAAVDSSVPRTAELAGITTLDDPLGHMPERFDAFICYCPSDI

SEQ ID NO: 22 - DNA, 516 bp, Homo sapiens
atggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgatctgcttaccaagctgggccgcgacgacgtgctgctggagctgggacccagcattgaggaggattgccaaaagtatatcttgaagcagcagcaggaggaggctgagaagcctttacaggtggccgctgtagacagcagtgtcccacggacagcagagctggcgggcatcaccacacttgatgaccccctggggcatatgcctgagcgtttcgatgccttcatctgctattgccccagcgacatc

SEQ ID NO: 23 - protein, 62 aa, Homo sapiens
KKVAKKPTNKAPHPKQEPQEINFPDDLPGSNTAAPVQETLHGCQPVTQEDGKESRISVQERQ

SEQ ID NO: 24 - DNA, 186 bp, Homo sapiens
aaaaaggtggccaagaagccaaccaataaggccccccaccccaagcaggagccccaggagatcaattttcccgacgatcttcctggctccaacactgctgctccagtgcaggagactttacatggatgccaaccggtcacccaggaggatggcaaagagagtcgcatctcagtgcaggagagacag

SEQ ID NO: 25 - protein, 6 aa, Artificial Sequence
  Description of Artificial Sequence: Synthetic peptide
MLEMLE

SEQ ID NO: 26 - DNA, 18 bp, Artificial Sequence
  Description of Artificial Sequence: Synthetic oligonucleotide
atgctcgagatgctggag

SEQ ID NO: 27 - protein, 108 aa, Homo sapiens
MGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 28 - DNA, 327 bp, Homo sapiens
atgggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatga

SEQ ID NO: 29 - protein, 296 aa, Homo sapiens
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRLIPIKYKAMKKEFPSILRFITVCDYTNPCTKSWFWTRLAKALSLP

SEQ ID NO: 30 - DNA, 891 bp, Homo sapiens
atggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgagctgcttaccaagctgggccgcgacgacgtgctgctggagctgggacccagcattgaggaggattgccaaaagtatatcttgaagcagcagcaggaggaggctgagaagcctttacaggtggccgctgtagacagcagtgtcccacggacagcagagctggcgggcatcaccacacttgatgaccccctggggcatatgcctgagcgtttcgatgccttcatctgctattgccccagcgacatccagtttgtgcaggagatgatccggcaactggaacagacaaactatcgactgaagttgtgtgtgtctgaccgcgatgtcctgcctggcacctgtgtctggtctattgctagtgagctcatcgaaaagaggtgccgccggatggtggtggttgtctctgatgattacctgcagagcaaggaatgtgacttccagaccaaatttgcactcagcctctctccaggtgcccatcagaagcgactgatccccatcaagtacaaggcaatgaagaaagagttccccagcatcctgaggttcatcactgtctgcgactacaccaacccctgcaccaaatcttggttctggactcgccttgccaaggccttgtccctgccctga

SEQ ID NO: 31 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 32 - DNA, 834 bp, Homo sapiens
atggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagagccctggtggtgatccccatcatcttcgggatcctgtttgccatcctcttggtgctggtctttatcaaaaaggtggccaagaagccaaccaataaggccccccaccccaagcaggaaccccaggagatcaattttcccgacgatcttcctggctccaacactgctgctccagtgcaggagactttacatggatgccaaccggtcacccaggaggatggcaaagagagtcgcatctcagtgcaggagagacagtga

SEQ ID NO: 33 - protein, 194 aa, Homo sapiens
MKWKALFTAAILQAQLPITASSLPHPTQQSPEKKVLGPGGCTCRHNRFCNEAQSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - DNA, 585 bp, Homo sapiens
atgaagtggaaggcgcttttcaccgcggccatcctgcaggcacagttgccgattacagcctccagcctcccccacccaactcagcagagccctgagaagaaagtcctgggtcccggaggctgcacctgcagacacaacagattctgcaatgaggcacagagctttggcctgctggatcccaaactctgctacctgctggatggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 35 - protein, 3 aa, Artificial Sequence
  Description of Artificial Sequence: Synthetic peptide
MLE

SEQ ID NO: 36 - DNA, 9 bp, Artificial Sequence
  Description of Artificial Sequence: Synthetic oligonucleotide
atgctcgag

SEQ ID NO: 37 - protein, 15 aa, Artificial Sequence
  Description of Artificial Sequence: Synthetic peptide
GGGGSGGGGSGGGGS

SEQ ID NO: 38 - DNA, 45 bp, Artificial Sequence
  Description of Artificial Sequence: Synthetic oligonucleotide
ggcggtggaggctccggtggaggcggctctggaggaggaggttca

SEQ ID NO: 39 - protein, 333 aa, Homo sapiens
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 40 - DNA, 1002 bp, Homo sapiens
atgccccctcctagactgctgtttttcctgctctttctcaccccaatggaagttagacctgaggaaccactggtcgttaaagtggaagaaggtgataatgctgtcctccaatgccttaaagggaccagcgacggaccaacgcagcaactgacttggagccgggagtcccctctcaagccgtttctcaagctgtcacttggcctgccaggtcttggtattcacatgcgcccccttgccatttggctcttcatattcaatgtgtctcaacaaatgggtggattctacctttgccagcccggccccccttctgagaaagcttggcagcctggatggaccgtcaatgttgaaggctccggtgagctgtttagatggaatgtgagcgaccttggcggactcggttgcggactgaaaaataggagctctgaaggaccctcttctccctccggtaagttgatgtcacctaagctgtacgtgtgggccaaggaccgccccgaaatctgggagggcgagcctccatgcctgccgcctcgcgattcactgaaccagtctctgtcccaggatctcactatggcgcccggatctactctttggctgtcttgcggcgttcccccagatagcgtgtcaagaggacctctgagctggacccacgtacaccctaagggccctaagagcttgttgagcctggaactgaaggacgacagacccgcacgcgatatgtgggtaatggagaccggccttctgctccctcgcgctaccgcacaggatgcagggaaatactactgtcatagagggaatctgactatgagctttcatctcgaaattacagcacggcccgttctttggcattggctcctccggactggaggctggaaggtgtctgccgtaacactcgcttacttgattttttgcctgtgtagcctggttgggatcctgcatcttcagcgagcccttgtattgcgccgaaaaagaaaacgaatgactgaccctacacgacgattctga

SEQ ID NO: 41 - protein, 107 aa, Homo sapiens; identical to SEQ ID NO: 1

SEQ ID NO: 42 - DNA, 321 bp, Homo sapiens
ggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaa

SEQ ID NO: 43 - protein, 106 aa, Homo sapiens
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKL

SEQ ID NO: 44 - DNA, 318 bp, Homo sapiens
ggcgtccaagtcgaaaccattagtcccggcgatggcagaacatttcctaaaaggggacaaacatgtgtcgtccattatacaggcatgttggaggacggcaaaaaggtggacagtagtagagatcgcaataaacctttcaaattcatgttgggaaaacaagaagtcattaggggatgggaggagggcgtggctcaaatgtccgtcggccaacgcgctaagctcaccatcagccccgactacgcatacggcgctaccggacatcccggaattattccccctcacgctaccttggtgtttgacgtcgaactgttgaagctc

SEQ ID NO: 45 - protein, 107 aa, Homo sapiens
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 46 - DNA, 321 bp, Homo sapiens
ggcgtgcaagtggaaactataagcccgggagacggccgcacatttcccaagagaggccagacctgcgtggtgcactatacaggaatgctggaggacgggaagaaattcgatagctcccgggatcgaaataagcctttcaaattcatgctgggcaagcaagaagtcatcagaggctgggaagaaggcgtcgcccagatgtccgtgggtcagcgcgccaagctgacaattagtccagattacgcctatggcgcaacaggccatcccggcatcatccccccacatgccacactcgtctttgatgtcgagctcctgaaactggag

SEQ ID NO: 47 - protein, 296 aa, Homo sapiens; identical to SEQ ID NO: 29

SEQ ID NO: 48 - DNA, 888 bp, Homo sapiens
atggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgagctgcttaccaagctgggccgcgacgacgtgctgctggagctgggacccagcattgaggaggattgccaaaagtatatcttgaagcagcagcaggaggaggctgagaagcctttacaggtggccgctgtagacagcagtgtcccacggacagcagagctggcgggcatcaccacacttgatgaccccctggggcatatgcctgagcgtttcgatgccttcatctgctattgccccagcgacatccagtttgtgcaggagatgatccggcaactggaacagacaaactatcgactgaagttgtgtgtgtctgaccgcgatgtcctgcctggcacctgtgtctggtctattgctagtgagctcatcgaaaagaggtgccgccggatggtggtggttgtctctgatgattacctgcagagcaaggaatgtgacttccagaccaaatttgcactcagcctctctccaggtgcccatcagaagcgactgatccccatcaagtacaaggcaatgaagaaagagttccccagcatcctgaggttcatcactgtctgcgactacaccaacccctgcaccaaatcttggttctggactcgccttgccaaggccttgtccctgccc

SEQ ID NO: 49 - protein, 106 aa, Artificial Sequence
  Description of Artificial Sequence: Synthetic polypeptide
DIQLTQSPSTLSASMGDRVTITCSASSSVRFIHWYQQKPGKAPKRLIYDTSKLASGVPSRFSGSGSGTDFTLTISSLQPEDFATYYCQQWGSSPFTFGQGTKVEIK

SEQ ID NO: 50 - DNA, 318 bp, Artificial Sequence
  Description of Artificial Sequence: Synthetic polynucleotide
gacatccaactgacgcaaagcccatctacactcagcgctagcatgggggacagggtcacaatcacgtgctctgcctcaagttccgttaggtttatccattggtatcagcagaaacctggaaaggccccaaaaagactgatctatgataccagcaagctggcttccggagtgccctcaaggttctcaggatctggcagtgggaccgatttcaccctgacaattagcagccttcagccagaggatttcgcaacctattactgtcagcaatgggggtccagcccattcactttcggccaaggaacaaaggtggagataaaa

SEQ ID NO: 51 - protein, 112 aa, Artificial Sequence
  Description of Artificial Sequence: Synthetic polypeptide
EVQLVEYGGGLVQPGGSLRLSCAASGFNIKDYYIHWVRQAPGKGLEWVAWIDPENGDTEFVPKFQGRATMSADTSKNTAYLQMNSLRAEDTAVYYCKTGGFWGQGTLVTVSS

SEQ ID NO: 52 - DNA, 336 bp, Artificial Sequence
  Description of Artificial Sequence: Synthetic polynucleotide
gaggtgcagctcgtggagtatggcgggggcctggtgcagcctgggggtagtctgaggctctcctgcgctgcctctggctttaacattaaagactactacatacattgggtgcggcaggccccaggcaaagggctcgaatgggtggcctggattgaccctgagaatggtgacactgagtttgtccccaagtttcagggcagagccaccatgagcgctgacacaagcaaaaacactgcttatctccaaatgaatagcctgcgagctgaagatacagcagtctattactgcaagacgggaggattctggggccagggaactctggtgacagttagttcc

SEQ ID NO: 53 - protein, 106 aa, Artificial Sequence
  Description of Artificial Sequence: Synthetic polypeptide
DIQLTQSPSSLSASVGDRVTITCSASSSVRFIHWYQQKPGKAPKRLIYDTSKLASGVPSRFSGSGSGTSYTLTISSLQPEDFATYYCQQWSSSPFTFGQGTKVEIK

SEQ ID NO: 54 - DNA, 318 bp, Artificial Sequence
  Description of Artificial Sequence: Synthetic polynucleotide
gacatccagctgacacaaagtcccagtagcctgtcagccagtgtcggcgatagggtgacaattacatgctccgcaagtagtagcgtcagattcatacactggtaccagcagaagcctgggaaggccccaaagaggcttatctacgataccagtaaactcgcctctggagttcctagccggttttctggatctggcagcggaactagctacaccctcacaatctccagtctgcaaccagaggactttgcaacctactactgccagcaatggagcagctcccctttcacctttgggcagggtactaaggtggagatcaag

SEQ ID NO: 55 - protein, 112 aa, Artificial Sequence
  Description of Artificial Sequence: Synthetic polypeptide
EVQLVESGGGLVQPGGSLRLSCAASGFNIKDYYIHWVRQAPGKGLEWIGWIDPENGDTEFVPKFQGKATMSADTSKNTAYLQMNSLRAEDTAVYYCKTGGFWGQGTLVTVSS

SEQ ID NO: 56 - DNA, 336 bp, Artificial Sequence
  Description of Artificial Sequence: Synthetic polynucleotide
gaggtgcagcttgtagagagcgggggaggcctcgtacagccagggggctctctgcgcctgtcatgtgcagcttcaggattcaatataaaggactattacattcactgggtacggcaagctcccggtaagggcctggaatggatcggttggatcgaccctgaaaacggagatacagaatttgtgcccaagttccagggaaaggctaccatgtctgccgatacttctaagaatacagcataccttcagatgaattctctccgcgccgaggacacagccgtgtattattgtaaaacgggagggttctggggtcagggtacccttgtgactgtgtcttcc

SEQ ID NO: 57 - protein, 139 aa, Artificial Sequence
  Description of Artificial Sequence: Synthetic polypeptide
GLQCVAAPRWVLSQVQLQQWGAGLLKPSETLSLTCAVYGGSFSGNYWSWIRQPPGKGLEWIGEINHSGSTNYNPSLKSRVTISVDTSKNQFSLKLSSVTAADTAVYYCARGGSYNYFDYWGQGTLVTVSSASTKGPSVK

SEQ ID NO: 58 - DNA, 417 bp, Artificial Sequence
  Description of Artificial Sequence: Synthetic polynucleotide
ggtctgcagtgtgtggcagcacccagatgggtcctgtcccaggtgcagctacagcagtggggcgcaggactgttgaagccttcggagaccctgtccctcacctgcgctgtctatggtgggtccttcagtggtaactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtgtattactgtgcgagaggggggagctacaactactttgactactggggccagggaaccctggtcaccgtctcctcagcctccaccaagggcccatcggtcaag

SEQ ID NO: 59 - protein, 239 aa, Artificial Sequence
  Description of Artificial Sequence: Synthetic polypeptide
MRLPAQLLGLLMLWVSGSSGDIVMTQSPLSLPVTPGEPASISCRSSQSLLHSNGYNYLVWYLQKPGQSPQLLIYLGSIRASGVPDRFSGSGSGTDFTLKISRVEAEDVGVYYCMQPLQTPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - DNA, 968 bp, Artificial Sequence
  Description of Artificial Sequence: Synthetic polynucleotide
aaagatcaggactcctcagttcaccttctcacaatgaggctccctgctcagctcctggggctgctaatgctctgggtctctggatccagtggggatattgtgatgactcagtctccactctccctgcccgtcacccctggagagccggcctccatctcctgcaggtctagtcagagcctcctacatagtaatggatacaactatttggtttggtacctgcagaagccaggacagtctccacagctcctgatctatttgggttctattcgggcctccggggtccctgacaggttcagtggcagtggatcaggcacagattttacactgaaaatcagcagagtggaggctgaggatgttggggtttattactgcatgcaacctctacaaactccgatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagaagtgcccccacctgctcctcagttccagcctgaccccctcccatcctttggcctctgaccctttttccacaggggacctacccctattgcggtcctccagctcatctttcacctcacccccctcctcctccttggctttaattatgctaatgttggaggagaatgaataaataaagtgaatctttgcaaaaaaaaaaaaaaaaaaaaa

SEQ ID NO: 61 - protein, 149 aa, Artificial Sequence
  Description of Artificial Sequence: Synthetic polypeptide
QLQESGPGLVKPSQTLSLTCTVSGGSISSGGYYWSWIRQHPGKGLEWIGYIYYSGSTYYNPSLKSRVTISVDTSKNQFSLKLSSGTAADTAVYYCARDHITMVRGVPKGMDVWGQGTTVTVSSASTKGPSVFPLAPSSKSTSGGTAALG

SEQ ID NO: 62 - DNA, 447 bp, Artificial Sequence
  Description of Artificial Sequence: Synthetic polynucleotide
cagctgcaggagtcgggcccaggactggtgaagccttcacagaccctgtccctcacctgcactgtctctggtggctccatcagcagtggtggttactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgggactgccgcggacacggccgtgtattactgtgcgagagaccacattactatggttcggggagtccccaagggcatggacgtctggggccaagggaccacggtcaccgtctcctcagcctccaccaagggcccatcggtcttccccctggcaccctcctccaagagcacctctgggggcacagcggccctgggc

SEQ ID NO: 63 - protein, 147 aa, Artificial Sequence
  Description of Artificial Sequence: Synthetic polypeptide
QLTQSPSSLSASVGDRVTITCRASQSISRHLNWYQQKPGKAPKFLIYVASSLQSGVPSRFSGSGSGTDFTLTISSLQPEDFATYFCQQSYSIPRTFGQGTKVEIKRTVAAPSVFIFPPSDEQLKSGTASVVCLLNNFYPREAKVQWK

SEQ ID NO: 64 - DNA, 441 bp, Artificial Sequence
  Description of Artificial Sequence: Synthetic polynucleotide
cagctgactcagtctccatcctccctgtctgcatctgtaggagacagagtcaccatcacttgccgggcaagtcagagcattagcaggcatttaaattggtatcagcagaaaccagggaaagcccctaagttcctgatctatgttgcatccagtttgcaaagtggggtcccatcaagattcagtggcagtggatctgggacagatttcactctcaccatcagcagtctgcaacctgaagattttgcaacttacttctgtcaacagagttacagtatcccccggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaag

SEQ ID NO: 65 - protein, 239 aa, Artificial Sequence
  Description of Artificial Sequence: Synthetic polypeptide
DIQLTQSPSSLSASVGDRVTITCSASSSVRFIHWYQQKPGKAPKRLIYDTSKLASGVPSRFSGSGSGTDFTLTISSLQPEDFATYYCQQWSSSPFTFGQGTKVEIKGSTSGGGSGGGSGGGGSSEVQLVESGGGLVQPGGSLRLSCAASGFNIKDYYIHWVRQAPGKGLEWVAWIDPENGDTEFVPKFQGRATISADTSKNTAYLQMNSLRAEDTAVYYCKTGGFWGQGTLVTVSSAAG

SEQ ID NO: 66 - skipped

SEQ ID NO: 67 - protein, 239 aa, Artificial Sequence
  Description of Artificial Sequence: Synthetic polypeptide
DIQLIQSPSSLSASVGDRVTITCSASSSVRFIHWYQQKPGKAPKRLIYDTSKLASGVPSRFSGSGSGTDFTLTISSLQPEDFATYYCQQWSSSPFTFGQGTKVEIKGSTSGGGSGGGSGGGGSSEVQLVESGGGLVQPGGSLRLSCAASGFNIKDYYIHWVRQAPGKGLEWVAWIDPENGDTEFVPKFQGRATISADTSKNTVYLQMNSLRAKDTAVYYCKTGGFWGQGTLVTVSSAAG

SEQ ID NO: 68 - skipped

SEQ ID NO: 69 - protein, 238 aa, Artificial Sequence
  Description of Artificial Sequence: Synthetic polypeptide
DIQLTQSPSSLSASVGDRVTITCSASSSVRFIHWYQQKPGKAPKRLIYDTSKLASGVPSRFSGSGSGTDFTLTISSLQPEDFATYYCQQWGSSPFTFGQGTKVEIKGSTSGGGSGGGSGGGGSEVQLVESGGGLVQPGGSLRLSCAASGFNIKDYYIHWVRQAPGKGLEWVAWIDPEYGDSEFVPKFQGRATMSADTSKNTAYLQMNSLRAEDTAVYYCKTGGFWGRGTLVTVSSAAG

SEQ ID NO: 70 - skipped

SEQ ID NO: 71 - protein, 239 aa, Artificial Sequence
  Description of Artificial Sequence: Synthetic polypeptide
DIQLTQSPSTLSASMGDRVTITCSASSSVRFIHWYQQKPGKAPKRLIYDTSKLASGVPSRFSGSGSGTDFTLTISSLQPEDFATYYCQQWGSSPFTFGQGTKVEIKGSTSGGGSGGGSGGGGSSEVQLVEYGGGLVQPGGSLRLSCAASGFNIKDYYIHWVRQAPGKGLEWVAWIDPENGDTEFVPKFQGRATMSADTSKNTAYLQMNSLRAEDTAVYYCKTGGFWGQGTLVTVSSAAG

(19) United States
(12) Patent Application Publication
(10) Pub. No.: US 2022/0062338 A1
(43) Pub. Date: Mar. 3, 2022

(54) PSCA CAR-T CELLS

(71) Applicant: BELLICUM PHARMACEUTICALS, INC., Houston, TX (US)

(72) Inventors: Paul WOODARD, Danville, CA (US); Aaron Edward FOSTER, Houston, TX (US); David Michael SPENCER, Frisco, TX (US); Olivia GARDNER, San Francisco, CA (US); William GROSSMAN, Hayward, CA (US); Matthew Robert COLLINSON-PAUTZ, Houston, TX (US); Annemarie MOSELEY, Dallas, TX (US)

(21) Appl. No.: 17/312,901

(22) PCT Filed: Dec. 12, 2019
(86) PCT No.: PCT/US2019/065901

§ 371 Date: Jun. 10, 2021

Related U.S. Application Data
(60) Provisional application No. 62/779,394, filed on Dec. 13, 2018.

(51) Int. Cl. A61K 35/17; A61K 31/4545; C12N 5/0783; C07K 14/725; C07K 14/705; C12N 9/90; C07K 16/30; A61P 35/00

(52) U.S. Cl. CPC A61K 35/17; A61K 31/4545; C12N 5/0636; C07K 14/7051; C07K 14/70578; C07K 2319/02; C07K 16/3069; A61P 35/00; C07K 2319/03; C07K 2319/33; C07K 2317/622; C12N 9/90

(57) ABSTRACT

The technology relates generally to the field of immunology and relates in part to T cell compositions and methods for treating diseases and disorders associated with the presence of tumor cells that express prostate stem cell antigen.

### RELATED APPLICATION

This application claims priority to U.S. Provisional Application No. 62/779,394, filed Dec. 13, 2018, the disclosures of which is hereby incorporated by reference in their entirety.

### SEQUENCE LISTING

The instant application contains a Sequence Listing which has been submitted electronically in ASCII format and is hereby incorporated by reference in its entirety. Said ASCII copy, created on Dec. 4, 2019, is named 14562-068-228_SL.txt and is 61,765 bytes in size.

### FIELD

The technology relates generally to the field of immunology and relates in part to T cell compositions and methods for treating diseases and disorders associated with the presence of tumor cells that express prostate stem cell antigen.

### BACKGROUND

Prostate stem cell antigen (PSCA) is a glycosylphosphatidylinosital-anchored cell surface protein with 123 amino acids. While PSCA has low expression in normal epithelial cells of prostate, urinary bladder, kidney, skin, esophagus, stomach, ocular and placenta, PSCA is upregulated in cancers of the pancreas, prostate, stomach, ovary, and bladder (Abate-Daga D 2014 Hum Gene Ther 25: 1003-1012). The expression of PSCA is positively correlated with advanced clinical stage and metastasis in prostate cancers (Saeki N 2010 Cancer Res 16: 3533-3538).

PSCA represents a potential target for anticancer immunotherapy. The safety and clinical activity of PSCA-directed monoclonal antibodies (AGS-1C4D4 or AGS-PSCA) was evaluated in Phase I and II clinical trials for the treatment of advanced pancreatic (Wolpin B M 2013 Ann Oncol 24: 1792) and prostate cancers (Antonarakis E S 2012 Cancer Chemother Pharmacol 69: 763-771, Morris M J 2012 Ann Oncol 23: 2714-2719). In about 200 subjects, treatment with an anti-PSCA antibody showed low toxicity at doses up to 48 mg/kg and modest efficacy. This favorable safety profile was attributed to the limited expression of PSCA on normal tissue (Abate-Daga D 2014 Hum Gene Ther 25: 1003-1012), which may help to reduce on-target, off-tumor side effects. Together, these data provide clinical evidence that PSCA is a relevant target of interest for therapeutic intervention.

Inducible PSCA CAR-T cells may be a useful type of immunotherapy for the treatment of PSCA-expressing solid tumor malignancies. Preclinical data show enhanced T cell proliferation, persistence and in vivo antitumor activity compared to traditional CAR-T therapies (Foster A E 2017 Mol Ther 25: 2176-2188, Mata M 2017 Cancer Discov 7: 1306-1319). To date, meaningful clinical activity of traditional CAR-T cell immunotherapy has been limited to hematologic malignancies whereas efficacy in solid tumor indications may be limited by generally low CAR-T cell persistence and therefore a lack of sustained antitumor activity over time. The addition of an inducible “on” switch to anti-PSCA CAR-T cells provides a controlled activation and proliferation signal, thereby not only improving the persistence of modified T cells but also prolonging the potential for antitumor efficacy.

Certain embodiments are described further in the following description, examples, claims and drawings.

### SUMMARY OF DISCLOSURE

In one aspect, provided herein is a method for treating a human subject, wherein the subject has been diagnosed with a disease associated with the presence of one or more solid tumors expressing prostate stem cell antigen (PSCA), comprising: administering to the subject 0.3×10⁶ cells/kg to about 9×10⁶ cells/kg of modified T cells comprising: (i) a first polynucleotide encoding an inducible MyD88/CD40 polypeptide and (ii) a second polynucleotide encoding a prostate stem cell antigen chimeric antigen receptor (PSCA-CAR) polypeptide.

In some embodiments, the method for treating a human subject provided herein further comprises administering an unmodified polyclonal T cells in an amount between 0.1×10⁶ cells/kg to 30×10⁶ cells/kg. In certain embodiments, the subject is administered an infusion of a heterogeneous T cell population comprising the modified and the unmodified T cells, wherein about 20% to about 90% of the T cells are modified T cells. In some embodiments, the subject is administered an infusion of a heterogeneous T cell population comprising the modified and the unmodified T cells, wherein about 20% to about 80% of the T cells are modified T cells. In some embodiments, the subject is administered an infusion of a heterogeneous T cell population comprising the modified and the unmodified T cells, wherein about 20% to about 70% of the T cells are modified T cells. In some embodiments, the subject is administered an infusion of a heterogeneous T cell population comprising the modified and the unmodified T cells, wherein about 20% to about 60% of the T cells are modified T cells. In some embodiments, the subject is administered an infusion of a heterogeneous T cell population comprising the modified and the unmodified T cells, wherein about 20% to about 50% of the T cells are modified T cells.

In certain embodiments, the T cells are administered to the subject in a diluent suitable for infusion into the subject comprising about 20×10⁶ to 120×10⁶ cells per milliliter.

In some embodiments, the method for treating a human subject provided herein further comprises administering to the subject a plurality of doses of rimiducid. In certain embodiments, rimiducid is administered to the subject once per day. In other embodiments, rimiducid is administered to the subject three times per week. In yet other embodiments, rimiducid is administered to the subject twice per week. In some embodiments, rimiducid is administered to the subject once per week. In some embodiments, rimiducid is administered to the subject once every other week. In other embodiments, rimiducid is administered to the subject twice per month.

In certain embodiments, each administration of rimiducid to the subject is about 0.4 mg/kg subject body weight. In specific embodiments, rimiducid is administered to the subject once per week, wherein each administration of rimiducid to the subject is about 0.4 mg/kg subject body weight. In other embodiments, rimiducid is administered to the subject once per day, wherein each administration of rimiducid to the subject is about 0.4 mg/kg subject body weight. In yet other embodiments, rimiducid is administered to the subject three times per week, wherein each administration of rimiducid to the subject is about 0.4 mg/kg subject body weight. In some embodiments, rimiducid is administered to the subject twice per week, wherein each administration of rimiducid to the subject is about 0.4 mg/kg subject body weight. In some embodiments, rimiducid is administered to the subject once every other week, wherein each administration of rimiducid to the subject is about 0.4 mg/kg subject body weight.

In some embodiments of any one of the methods for treating a human subject provided herein, the solid tumor is pancreatic cancer, gastric cancer or prostate cancer.

In some embodiments of any one of the methods for treating a human subject provided herein, the inducible MyD88/CD40 polypeptide comprises
i) two multimeric ligand binding regions;
ii) a MyD88 polypeptide or a truncated MyD88 polypeptide lacking a TIR domain; and
iii) a CD40 cytoplasmic polypeptide region lacking a CD40 extracellular domain.

In certain embodiments, each multimeric ligand binding region comprises an FKBP12 variant polypeptide. In some embodiments, each FKBP12 variant polypeptide comprises an amino acid substitution at position 36 that binds with higher affinity to the multimeric ligand than the wild type FKBP12 polypeptide. In specific embodiments, the amino acid substitution at position 36 is selected from the group consisting of valine, isoleucine, leucine, and alanine. In some embodiments, each multimeric ligand binding region is an FKB12v36 region.

In some embodiments, the truncated MyD88 polypeptide has the amino acid sequence of SEQ ID NO: 21, or a functional fragment thereof.

In certain embodiments, the CD40 cytoplasmic polypeptide comprises the amino acid sequence of SEQ ID NO: 23, or a functional fragment thereof.

In some embodiments of any one of the methods provided herein, the PSCA-CAR polypeptide comprises an antigen recognition moiety, a transmembrane region, and a T cell activation molecule. In certain embodiments, the antigen recognition moiety is derived from a PSCA antibody selected from the group consisting of 1G8, 2A2, 2H9, 3C5, 3E6, 3G3, 4A10, and A11, wherein 1G8, 2A2, 2H9, 3C5, 3E6, 3G3, and 4A10 are produced by the hybridomas designated HB-12612, HB-12613, HB-12614, HB-12616, HB-12618, HB-12615, and HB-12617, respectively, as deposited with the American Type Culture Collection.

In some embodiments, the antigen recognition moiety comprises the complementarity determining regions (CDRs) of the heavy chain variable domain and the light chain variable domain of an amino acid sequence selected from the group consisting of SEQ ID NOs: 65, 67, 69, and 71.

In other embodiments, the antigen recognition moiety comprises a variable heavy chain amino acid sequence and a variable light chain amino acid sequence selected from the group consisting of SEQ ID NOs: 51 and 49; SEQ ID NOs: 55 and 53; SEQ ID NOs: 57 and 59; and SEQ ID NOs: 61 and 63.

In yet other embodiments, the antigen recognition moiety is a single-chain variable (scFV) comprising a variable light chain amino acid sequence of SEQ ID NO: 49 and a variable heavy chain amino acid sequence of SEQ ID NO: 51.

In some embodiments, the antigen recognition moiety is a scFV comprising a variable light chain amino acid sequence of SEQ ID NO:53 and a variable heavy chain amino acid sequence of SEQ ID NO:55.

In other embodiments, the antigen recognition moiety is a scFV comprising a variable light chain amino acid sequence of SEQ ID NO: 57 and a variable heavy chain amino acid sequence of SEQ ID NO: 59.

In yet other embodiments, the antigen recognition moiety is a scFV comprising a variable light chain amino acid sequence of SEQ ID NO: 61 and a variable heavy chain amino acid sequence of SEQ ID NO: 63.

In some embodiments, the antigen recognition moiety is a scFV comprising a variable light chain amino acid sequence of SEQ ID NO: 65 and a variable heavy chain amino acid sequence of SEQ ID NO: 67.

In other embodiments, the antigen recognition moiety is a scFV comprising a variable light chain amino acid sequence of SEQ ID NO: 69 and a variable heavy chain amino acid sequence of SEQ ID NO: 71.

In another aspect, provided herein is a heterogeneous T cell population comprising modified and unmodified T cells, wherein the modified T cells comprise a) a first polynucleotide encoding an inducible MyD88/CD40 polypeptide and b) a second polynucleotide encoding a PSCA-CAR polypeptide.

In some embodiments, about 20% to about 90% of the T cells are modified T cells. In some embodiments, about 20% to about 80% of the T cells are modified T cells. In some embodiments, about 20% to about 70% of the T cells are modified T cells. In certain embodiments, about 20% to about 60% of the T cells are modified T cells. In some embodiments, about 20% to about 50% of the T cells are modified T cells.

In some embodiments, the T cell population is suspended in freezing medium. In certain embodiments, the T cell population provided herein is a cryopreserved T cell population.

In some embodiments, the T cell population provided herein comprises about 20×10⁶ to 150×10⁶ cells per milliliter in a diluent suitable for infusion into a subject. In other embodiments, the T cell population provided herein comprises about 20×10⁶ to 120×10⁶ cells per milliliter in a diluent suitable for infusion into a subject.

In some embodiments, the T cell population provided herein comprises modified T cells comprising a first polynucleotide encoding an inducible MyD88/CD40 polypeptide, and the inducible MyD88/CD40 polypeptide comprises
i) two multimeric ligand binding regions;
ii) a MyD88 polypeptide or a truncated MyD88 polypeptide lacking a TIR domain; and
iii) a CD40 cytoplasmic polypeptide region lacking a CD40 extracellular domain.

In some embodiments, each multimeric ligand binding region comprises an FKBP12 variant polypeptide. In other embodiments, each FKBP12 variant polypeptide binds with higher affinity to the multimeric ligand than the wild type FKBP12 polypeptide. In certain embodiments, each FKBP12 variant polypeptide comprises an amino acid substitution at position 36 that binds with higher affinity to the multimeric ligand than the wild type FKBP12 polypeptide. In some embodiments, the amino acid substitution at position 36 is selected from the group consisting of valine, isoleucine, leucine, and alanine. In some embodiments, each multimeric ligand binding region is an FKB12v36 region.

In some embodiments, the T cell population provided herein comprises modified T cells comprising a first polynucleotide encoding an inducible MyD88/CD40 polypeptide, and the inducible MyD88/CD40 polypeptide comprises
i) two multimeric ligand binding regions;
ii) a MyD88 polypeptide or a truncated MyD88 polypeptide lacking a TIR domain; and
iii) a CD40 cytoplasmic polypeptide region lacking a CD40 extracellular domain,
   and the truncated MyD88 polypeptide has the amino acid sequence of SEQ ID NO: 21, or a functional fragment thereof. In specific embodiments, the truncated MyD88 polypeptide consists of the amino acid sequence of SEQ ID NO: 21.

In some embodiments, the T cell population provided herein comprises modified T cells comprising a first polynucleotide encoding an inducible MyD88/CD40 polypeptide, and the inducible MyD88/CD40 polypeptide comprises
i) two multimeric ligand binding regions;
ii) a MyD88 polypeptide or a truncated MyD88 polypeptide lacking a TIR domain; and
iii) a CD40 cytoplasmic polypeptide region lacking a CD40 extracellular domain,
   and the CD40 cytoplasmic polypeptide comprises the amino acid sequence of SEQ ID NO: 23, or a functional fragment thereof. In some embodiments, the CD40 cytoplasmic polypeptide consists of the amino acid sequence of SEQ ID NO: 23, or a functional fragment thereof.

In some embodiments, provided herein is a heterogeneous T cell population comprising modified and unmodified T cells, wherein the modified T cells comprise a) a first polynucleotide encoding an inducible MyD88/CD40 polypeptide and b) a second polynucleotide encoding a PSCA-CAR polypeptide, and the PSCA-CAR polypeptide comprises an antigen recognition moiety, a transmembrane region, and a T cell activation molecule. In certain embodiments, the antigen recognition moiety is derived from a PSCA antibody selected from the group consisting of 1G8, 2A2, 2H9, 3C5, 3E6, 3G3, 4A10, and A11, wherein 1G8, 2A2, 2H9, 3C5, 3E6, 3G3, and 4A10 are produced by the hybridomas designated HB-12612, HB-12613, HB-12614, HB-12616, HB-12618, HB-12615, and HB-12617, respectively, as deposited with the American Type Culture Collection.

In some embodiments, the antigen recognition moiety comprises the complementarity determining regions (CDRs) of the heavy chain variable domain and the light chain variable domain of an amino acid sequence selected from the group consisting of SEQ ID NOs: 65, 67, 69, and 71. In other embodiments, the antigen recognition moiety comprises a variable heavy chain amino acid sequence and a variable light chain amino acid sequence selected from the group consisting of SEQ ID NOs: 51 and 49; SEQ ID NOs: 55 and 53; SEQ ID NOs: 57 and 59; and SEQ ID NOs: 61 and 63. In some embodiments, the antigen recognition moiety is a scFV comprising a variable light chain amino acid sequence of SEQ ID NO: 49 and a variable heavy chain amino acid sequence of SEQ ID NO: 51. In some embodiments, the antigen recognition moiety is a scFV comprising a variable light chain amino acid sequence of SEQ ID NO:53 and a variable heavy chain amino acid sequence of SEQ ID NO:55. In some embodiments, the antigen recognition moiety is a scFV comprising a variable light chain amino acid sequence of SEQ ID NO: 57 and a variable heavy chain amino acid sequence of SEQ ID NO: 59. In some embodiments, the antigen recognition moiety is a scFV comprising a variable light chain amino acid sequence of SEQ ID NO: 61 and a variable heavy chain amino acid sequence of SEQ ID NO: 63. In some embodiments, the antigen recognition moiety is a scFV comprising a variable light chain amino acid sequence of SEQ ID NO: 65 and a variable heavy chain amino acid sequence of SEQ ID NO: 67. In some embodiments, the antigen recognition moiety is a scFV comprising a variable light chain amino acid sequence of SEQ ID NO: 69 and a variable heavy chain amino acid sequence of SEQ ID NO: 71.

### BRIEF DESCRIPTION OF THE DRAWINGS

The drawings illustrate certain embodiments of the technology and are not limiting. For clarity and ease of illustration, the drawings are not made to scale and, in some instances, various aspects may be shown exaggerated or enlarged to facilitate an understanding of particular embodiments.

FIG. 1 shows PSCA expression in tumor tissues.

FIG. 2 shows a retroviral vector construct that includes an inducible MyD88/CD40 component and an anti-PSCA CAR component. Q, CD34.

FIG. 3 shows an illustration of inducible MyD88/CD40. MyD88 and CD40 signaling pathways are co-opted to generate inducible costimulation. IFN, interferon; iMC, inducible MyD88/CD40; TIR, toll/interleukin-1 receptor; TLR, toll-like receptor.

FIG. 4 shows an illustration of a T cell engineered with inducible costimulation (i.e., an inducible MyD88/CD40 PSCA CAR-T cell). Characteristics of the cell include: autologous T cell product candidate; antigen-specific activation through PSCA-CD3ζ CAR; and rimiducid-dependent costimulation through inducible MyD88/CD40 domain. CAR, chimeric antigen receptor; iMC, inducible MyD88/CD40; PSCA, prostate stem cell antigen; Rim, rimiducid; TCR, T cell receptor.

FIGS. 5A-5B show dose-dependent tumor killing by inducible MyD88/CD40 PSCA CAR-T cells in HPAC-bearing NSG mice. FIG. 5A: tumor volume measured over time following injection of non-transduced cells or inducible MyD88/CD40 PSCA CAR-T cells at the indicated doses. Red arrows designate rimiducid injections for applicable dose groups. FIG. 5B: survival following injection of non-transduced cells or inducible MyD88/CD40 PSCA CAR-T cells at the indicated doses. CID=rimiducid; NT=non-transduced T cells.

FIG. 6 shows data supporting a prostate stem cell antigen (PSCA) target rationale. GPI, glycosylphosphatidylinositol; hACTB, human beta-actin; LoQ, limit of quantification.

FIG. 7 shows an illustration of a cell dosing study design (i.e., using inducible MyD88/CD40 PSCA CAR-T cells). Objectives for Part 1 of the study included: safety, tolerability, MTD and/or RDE for use in Part 2. MTD, maximum tolerated dose; PSCA, prostate stem cell antigen; RDE, recommended dose expansion.

FIG. 8 provides a table showing patient demographics and clinical characteristics. * Cytoxan 1 g/m² by IV infusion on Day −3; † PSCA (copies per 10⁶ copies hACTB) was measured at screening. CTX, cyclophosphamide; hACTB, human beta-actin; LD, lymphodepletion; PSCA, prostate stem cell antigen; Rim, rimiducid.

FIG. 9 shows a safety summary. No DLTs, neurotoxicity, or events of cytokine release syndrome were observed. Pyrexia was the only treatment-related AE reported by >1 patient (n=2) (grade 1-2 on Day 0 following infusion of inducible MyD88/CD40 PSCA CAR-T cells). Both events resolved within 24-36 hours with supportive care. AE=adverse event; DLT=dose-limiting toxicity.

FIG. 10 shows inducible MyD88/CD40 PSCA CAR-T cell expansion and persistence. Observations included: limited evidence of lymphodepletion (LD) with cyclophosphamide (CTX)-only regimen (79%±25% of cells remained); rapid cell expansion by Day 4, but no persistence without rimiducid; and with single-dose rimiducid, cell expansion of 3- to 20-fold within 7 days in 4 patients, and cell persistence of >3 weeks in 3 patients. * Vector copy number <limit of quantification. CTX, cyclophosphamide; LD, lymphodepletion; PSCA, prostate stem cell antigen; Rim, rimiducid

FIG. 11 shows peripheral cytokine profiles over time. Rim, rimiducid.

FIG. 12 shows evidence of anti-tumor activity in patients treated with inducible MyD88/CD40 PSCA CAR-T cells. Scans were performed at week 4 (month 1), week 8 (month 2), and every 8 weeks (every 2 months) thereafter until progression. CA19-9, cancer antigen 19-9; CR, complete response; PD, progressive disease; PR, partial response; PSCA, prostate stem cell antigen; SD, stable disease.

FIG. 13 shows evidence of anti-tumor activity in patients treated with inducible MyD88/CD40 PSCA CAR-T cells. PSCA, prostate stem cell antigen.

FIG. 14 shows time to next treatment after administration of inducible MyD88/CD40 PSCA CAR-T cells. PD, progressive disease; pseudo PD, pseudoprogression, progressive disease; SD, stable disease.

### DETAILED DESCRIPTION

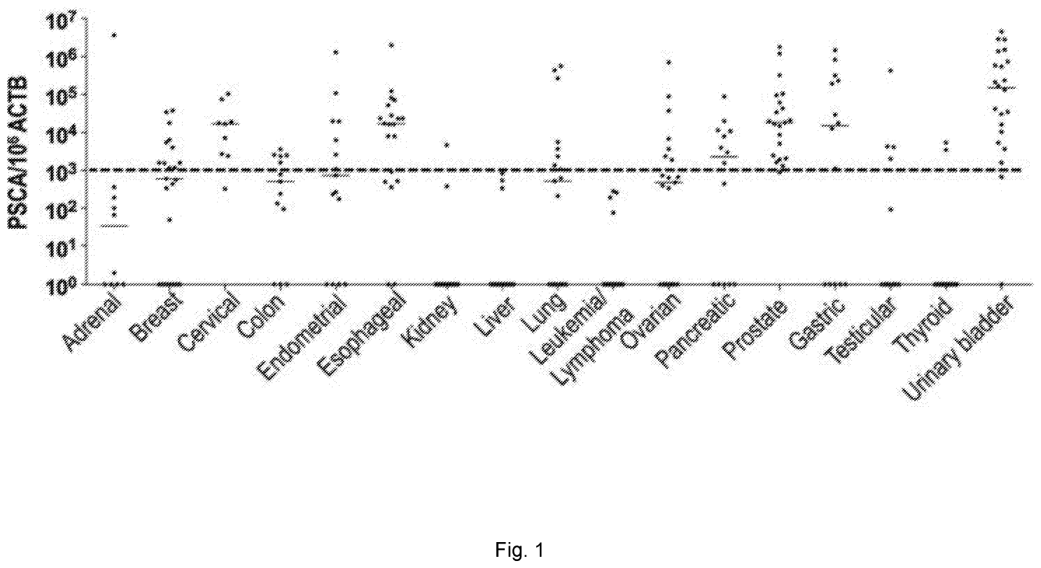

Prostate stem cell antigen (PSCA) is a glycosylphosphatidylinosital-anchored cell surface protein with 123 amino acids. While PSCA has low expression in normal epithelial cells of prostate, urinary bladder, kidney, skin, esophagus, stomach, ocular and placenta, PSCA is upregulated in cancers of the pancreas, prostate, stomach, ovary, and bladder (FIG. 1; Abate-Daga D 2014 Hum Gene Ther 25: 1003-1012.). The expression of PSCA is positively correlated with advanced clinical stage and metastasis in prostate cancers (Saeki N 2010 Cancer Res 16: 3533-3538).

PSCA-Expressing Solid Tumors with Significant Unmet Medical Need

Pancreatic Cancer

Pancreatic adenocarcinoma is the fourth most common cause of cancer-related death in the United States (Siegel R L 2017 CA Cancer J Clin 67: 7-30). Because survival is poor, the population distribution of those who die of pancreatic cancer is similar to that who are diagnosed with the disease. It is estimated that 55,440 people will be diagnosed with and approximately 44,330 people will die of pancreatic adenocarcinoma in 2018 (SEER Pancreatic Cancer 2018 Accessed 14 May 2018 from https://seer.cancer.gov/statfacts/html/prost.html). Radiographic imaging, liver function testing, and levels of circulating tumor biomarkers (CA 19-9) are the primary means used for diagnosis and staging.

Surgical resection is the only potentially curative therapy for managing resectable and borderline resectable pancreatic cancer. However, the disease is often difficult to detect in these early stages, and many patients are not diagnosed until the disease has already progressed to an unresectable state, becoming either locally advanced or with distant metastases. For subjects with previously untreated, locally advanced or metastatic disease and good performance status, combination chemotherapy remains the standard of care. Although randomized clinical trials have shown a survival advantage for both FOLFIRINOX and gemcitabine in combination with nab-paclitaxel as first-line therapy, median overall survival remains less than 12 months and treatment-related severe toxicity (notably myelosuppression and peripheral neuropathy) is common. These data highlight the need to develop safe and effective second-line therapies beyond current standards of care; the latter are limited to single-agent chemotherapy or palliative care (Conroy T 2011 N Engl J Med 364: 1817-1825, Von Hoff D D 2013 N Engl J Med 369: 1691-1703).

Prostate Cancer

Prostate cancer is the most common, noncutaneous cancer in men in the United States (Litwin M S 2017 JAMA 317: 2532-2542). In 2018, approximately 165,000 men will be diagnosed with prostate cancer, which is the second leading cause of cancer death in men with an estimated annual death rate of 29,000 (Siegel R L 2017 CA Cancer J Clin 67: 7-30). Diagnosis is typically made by ultrasound guided needle biopsy. The histological pattern is scored using the Gleason system.

For men with metastatic prostate cancer, androgen deprivation therapy (ADT) with or without docetaxel is the first line of treatment (Sweeney C J 2015 N Engl J Med 373:737-746., James N D 2016 Lancet 387: 1163-1177, Litwin M S 2017 JAMA 317: 2532-2542). In patients whose metastatic prostate cancer becomes unresponsive to ADT, other agents that block the androgen pathway (abiraterone, enzalutamide) may slow disease progression, improve survival, and improve quality of life (de Bono J S 2011 N Engl J Med 364: 1995-2005, Scher H 2012 N Engl J Med 367: 1187-1197, Ryan C J 2013 N Engl J Med 368:138-148., Beer T M 2014 N Engl J Med 371: 424-433). Other agents such as sipuleucel-T, an autologous cellular therapy (Kantoff P W 2010, Schellhammer P F 2013 Urology 81: 1297-1302), and cabazitaxel (de Bono J S 2011 N Engl J Med 364: 1995-2005), a taxane, may be incorporated into the treatment. Other agents such as denosomab, zoledronic acid, or 223Radium may be used for protection from bone-metastases and skeletal-related events (Beer T M 2014 N Engl J Med 371: 424-433, Parker C 2013 N Engl J Med 369: 1659-1660). While improvements have been made with the advent of these therapies, survival is limited for patients with recurrent metastatic prostate cancer. Overall survival rates for patients with recurrent disease following chemotherapy ranges from 14.0 to 18.4 months.

Gastric Cancer

Gastric cancer is the fourth most common cancer and second leading cause of cancer-related death worldwide. There are more than 950,000 new cases per year. In 2012 an estimated 720,000 patients died from gastric cancer (Ferlay J 2015 Int J Cancer 136: E359-E386). Gastric cancer is separated into gastric adenocarcinoma and gastroesophageal junction adenocarcinomas. Histologically, gastric cancer is separated into two main types, diffuse and intestinal. H. pylori infection is the most important cause of distal gastric cancer (Bornschein J 2010 Dig Dis 28: 609-614).

Therapy options for gastric cancer depend on the treatment setting and include surgery, radiotherapy, chemotherapy, and more recently targeted therapy with monoclonal antibodies as well as immunotherapy. For patients with metastatic gastric cancer, combination chemotherapy, specifically 5-fluorouracil plus platinum, has been established as the standard frontline treatment (Kim N K 1993 Cancer 71: 3813-3818, Ohtsu S 2003 J Clin Oncol 21: 54-59, Van Cutsem E 2006 J Clin Oncol 24: 4991-4997). While primary therapy objectives are survival prolongation, symptom palliation, and quality of life improvement, all patients eventually experience disease progression with or without clinically significant treatment-related toxicity. For those patients who go on to receive second- and later lines of treatment, results from clinical trials in Asia have demonstrated improved survival benefit for chemotherapy compared to best supportive care (Takashima A 2014 Gastric Cancer 17: 522-528). Despite these efficacy benefits, use of chemotherapy after the first-line is often limited to patients with good performance status. This highlights the importance of developing additional, later-line therapy options that not only improve treatment outcomes and prolong survival but also maintain quality of life of all patients with advanced gastric cancer.

PSCA-Specific CAR-T Cells

A PSCA-specific CAR-T cell (e.g., an inducible MyD88/CD40 PSCA CAR-T cell) is a PSCA-directed, genetically modified, T cell that binds to PSCA-expressing cells.

T cells bearing first generation chimeric antigen receptors (CARs), including a tumor antigen-specific, single-chain variable fragment (scFv) domain and the T cell receptor (TCR)-associated CD3ζ intracellular signaling molecule, fail to persist or expand in vivo (Kershaw, M. H. et al. (2006) Clinical cancer research: an official journal of the American Association for Cancer Research 12: 6106-6115; Pule, M. A. et al. (2008) Nature Med. 14: 1264-1270; Till, B. G. et al. (2012) Blood 119: 3940-3950) as tumor cells often lack the requisite costimulatory molecules necessary for complete T cell activation (Inman, B. A., et al. (2007) Current cancer drug targets 7: 15-30). Second generation CAR-T cells that incorporate potent intracellular costimulatory domains, like CD28 or 4-1BB (Carpenito, C. et al. (2009) Proc. Natl. Acad. Sci. USA 106: 3360-3365; Song, D. G. et al. (2012) Blood 119: 696-706), show improved survival and in vivo expansion following adoptive transfer (Kalos, M. et al. (2011) Science translational medicine 3:95ra73; Porter, D. L., et al. (2011) The New Eng. J. Med. 365: 725-733; Brentjens, R. J. et al. (2013) Science translational medicine 5:177ra138; Kochenderfer, J. N. et al. (2015) J. Clin. Oncol. 33(6): 540-549). T cells expressing CARs have shown long-term efficacy for the treatment of some types of cancer, however, toxicity associated with excessive T cell activation, such as cytokine release syndrome (CRS) remain a concern. Additionally, CAR-T cell efficacy has been more limited in solid tumors due to poor CAR-T cell survival, activation and proliferation, presumably due to the more profound inhibitory effects of the tumor microenvironment (Jena, B., et al. (2014) Current hematologic malignancy reports 9: 50-56; Dotti, G., et al. (2014) Immunological reviews 257: 107-126). Thus, strategies that allow controlled expansion and survival of tumor-targeted T cells would maximize therapeutic potency while minimizing toxicities.

Antitumor activity of CAR-T cells is associated with robust CAR-T cell expansion post-infusion that is often associated with toxicity (i.e., severe cytokine-release syndrome and neurotoxicity), while patients with poor CAR-T proliferation and persistence show reduced rates of durable remissions. In the Examples presented herein, it is demonstrated that controllable signaling from an inducible chimeric stimulating polypeptide, e.g., inducible MyD88/CD40 (iMC), can enhance CAR-T survival, proliferative capacity and antitumor activity.

T cells (also referred to as T lymphocytes) belong to a group of white blood cells referred to as lymphocytes. Lymphocytes generally are involved in cell-mediated immunity. The “T” in “T cells” refers to cells derived from or whose maturation is influenced by the thymus. T cells can be distinguished from other lymphocytes types such as B cells and Natural Killer (NK) cells by the presence of cell surface proteins known as T cell receptors. The term “activated T cells” as used herein, refers to T cells that have been stimulated to produce an immune response (e.g., clonal expansion of activated T cells) by recognition of an antigenic determinant, such as, for example, presented in the context of a Class II major histo-compatibility (MHC) marker. T cells are activated by the presence of an antigenic determinant, cytokines and/or lymphokines and cluster of differentiation cell surface proteins (e.g., CD3, CD4, CD8, the like and combinations thereof). Cells that express a cluster of differential protein often are said to be “positive” for expression of that protein on the surface of T cells (e.g., cells positive for CD3, CD4, or CD8 expression are referred to as CD3⁺, CD4⁺, or CD8⁺). CD3 and CD4 proteins are cell surface receptors or co-receptors that may be directly and/or indirectly involved in signal transduction in T cells.

T cells express receptors on their surfaces (i.e., T cell receptors) that recognize antigens presented on the surface of cells. During a normal immune response, binding of these antigens to the T cell receptor, in the context of MHC antigen presentation, initiates intracellular changes leading to T cell activation. Chimeric antigen receptors (CARs) are artificial receptors designed to convey antigen specificity to T cells without the requirement for MHC antigen presentation. They include an antigen-specific component, a transmembrane component, and an intracellular component selected to activate the T cell and provide specific immunity. Chimeric antigen receptor-expressing T cells may be used in various therapies, including cancer therapies.

By “chimeric antigen receptor” or “CAR” is meant, for example, a chimeric polypeptide that comprises a polypeptide sequence that recognizes a target antigen (an antigen-recognition domain, antigen recognition region, antigen recognition moiety, or antigen binding region) linked to a transmembrane polypeptide and intracellular domain polypeptide selected to activate the T cell and provide specific immunity. An antigen recognition domain may be any polypeptide or fragment thereof, such as, for example, an antibody fragment variable domain, either naturally-derived, or synthetic, which binds to an antigen. Examples of antigen recognition moieties include, but are not limited to, polypeptides derived from antibodies, such as, for example, single chain variable fragments (scFv), Fab, Fab′, F(ab′)2, and Fv fragments; polypeptides derived from T Cell receptors, such as, for example, TCR variable domains; polypeptides derived from Pattern Recognition Receptors, and any ligand or receptor fragment that binds to the extracellular cognate protein.

By “T cell activation molecule” is meant a polypeptide that, when incorporated into a T cell expressing a chimeric antigen receptor, enhances activation of the T cell. Examples include, but are not limited to, ITAM-containing, Signal 1 conferring molecules such as, for example, CD3 ζ (CD3 zeta; CD247) polypeptide, and Fc receptor gamma, such as, for example, Fc epsilon receptor gamma (FcεR1γ) subunit (Haynes, N. M., et al. J. Immunol. 166:182-7 (2001)). The intracellular domain may comprise at least one polypeptide which causes activation of the T cell, such as, for example, but not limited to, CD3 ζ.

In some embodiments, the basic components of a chimeric antigen receptor (CAR) include the following. The variable heavy (VH) and light (VL) chains for a tumor-specific monoclonal antibody are fused in-frame with the CD3 ζ chain (ζ) from the T cell receptor complex. The VH and VL are generally connected together using a flexible glycine-serine linker, and then attached to the transmembrane domain by a spacer (e.g., CD8a stalk or CH₂CH₃) to extend the scFv away from the cell surface so that it can interact with tumor antigens.

In some embodiments, PSCA-specific CAR-T cells target PSCA with a first-generation CAR construct containing a traditional CD3ζ cytoplasmic signaling domain together with a single-chain variable region domain (scFV) of a humanized PSCA antibody. In some embodiments, the anti-PSCA CAR is engineered to express an inducible dual co-stimulatory domain comprised of two FK binding proteins (FKBP) in-frame with the signaling domains from MyD88 and CD40 (inducible MyD88/CD40 or iMC).

In some embodiments, the single-chain variable region domain (scFV) may be derived from a PSCA antibody selected from the group consisting of 1G8, 2A2, 2H9, 3C5, 3E6, 3G3 and 4A10 produced by the hybridomas designated HB-12612, HB-12613, HB-12614, HB-12616, HB-12618, HB-12615, and HB-12617, respectively, as deposited with the American Type Culture Collection, and those discussed in, for example, U.S. Pat. No. 7,527,786, by Reiter, R. E., et al., issued May 5, 2009, published as US 2003-0113810 A1 on Jun. 19, 2003, which are all hereby incorporated by reference in their entireties.

In some embodiments, the single-chain variable region domain (scFV) may be derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, PSCA-specific CAR-T cells target PSCA with a first-generation CAR construct containing a traditional CD3ζ cytoplasmic signaling domain together with a single-chain variable region domain (scFV) containing the variable heavy chain and variable light chain A11 VL (SEQ ID NO: 49), and A11 VH (SEQ ID NO: 51), respectively. In some embodiments, the anti-PSCA CAR containing the variable heavy chain and variable light chain A11 VL (SEQ ID NO: 49), and A11 VH (SEQ ID NO: 51) is engineered to express an inducible dual co-stimulatory domain comprised of two FK binding proteins (FKBP) in-frame with the signaling domains from MyD88 and CD40 (inducible MyD88/CD40 or iMC).

In some embodiments, PSCA-specific CAR-T cells target PSCA with a first-generation CAR construct containing a traditional CD3ζ cytoplasmic signaling domain together with a single-chain variable region domain (scFV) containing the variable heavy chain and variable light chain bm2B3 VL (SEQ ID NO:53) and bm2B3 VH (SEQ ID NO:55), respectively. In some embodiments, the anti-PSCA CAR containing the variable heavy chain and variable light chain bm2B3 VL (SEQ ID NO:53) and bm2B3 VH (SEQ ID NO:55) is engineered to express an inducible dual co-stimulatory domain comprised of two FK binding proteins (FKBP) in-frame with the signaling domains from MyD88 and CD40 (inducible MyD88/CD40 or iMC).

In some embodiments, PSCA-specific CAR-T cells target PSCA with a first-generation CAR construct containing a traditional CD3ζ cytoplasmic signaling domain together with a single-chain variable region domain (scFV) containing the variable heavy chain and variable light chain HA1-4.121 VH (SEQ ID NO: 57) and HLA1-4.121 VL (SEQ ID NO: 59), respectively. In some embodiments, the anti-PSCA CAR containing the variable heavy chain and variable light chain HA1-4.121 VH (SEQ ID NO: 57) and HLA1-4.121 VL (SEQ ID NO: 59) is engineered to express an inducible dual co-stimulatory domain comprised of two FK binding proteins (FKBP) in-frame with the signaling domains from MyD88 and CD40 (inducible MyD88/CD40 or iMC).

In some embodiments, PSCA-specific CAR-T cells target PSCA with a first-generation CAR construct containing a traditional CD3ζ cytoplasmic signaling domain together with a single-chain variable region domain (scFV) containing the variable heavy chain and variable light chain H1-1.10 VH (SEQ ID NO: 61) and H1-1.10 VL (SEQ ID NO: 63), respectively. In some embodiments, the anti-PSCA CAR containing the variable heavy chain and variable light chain H1-1.10 VH (SEQ ID NO: 61) and H1-1.10 VL (SEQ ID NO: 63) is engineered to express an inducible dual co-stimulatory domain comprised of two FK binding proteins (FKBP) in-frame with the signaling domains from MyD88 and CD40 (inducible MyD88/CD40 or iMC).

In some embodiments, PSCA-specific CAR-T cells target PSCA with a first-generation CAR construct containing a traditional CD3ζ cytoplasmic signaling domain together with a single-chain variable region domain (scFV) containing the variable heavy chain and variable light chain 1G8 (2B3) (SEQ ID NO: 65) and 1G8 (2B3-05) (SEQ ID NO: 67), respectively. In some embodiments, the anti-PSCA CAR containing the variable heavy chain and variable light chain 1G8 (2B3) (SEQ ID NO: 65) and 1G8 (2B3-05) (SEQ ID NO: 67) is engineered to express an inducible dual co-stimulatory domain comprised of two FK binding proteins (FKBP) in-frame with the signaling domains from MyD88 and CD40 (inducible MyD88/CD40 or iMC).

In some embodiments, PSCA-specific CAR-T cells target PSCA with a first-generation CAR construct containing a traditional CD3ζ cytoplasmic signaling domain together with a single-chain variable region domain (scFV) containing the variable heavy chain and variable light chain 1G8 (2B3-A2) (SEQ ID NO: 69) and 1G8 (2B3-A11) (SEQ ID NO: 71), respectively. In some embodiments, the anti-PSCA CAR containing the variable heavy chain and variable light chain 1G8 (2B3-A2) (SEQ ID NO: 69) and 1G8 (2B3-A11) (SEQ ID NO: 71) is engineered to express an inducible dual co-stimulatory domain comprised of two FK binding proteins (FKBP) in-frame with the signaling domains from MyD88 and CD40 (inducible MyD88/CD40 or iMC).

Costimulation

In some embodiments, provided herein are compositions and methods comprising a CAR-T cell population comprising an inducible chimeric stimulating polypeptide. An inducible chimeric stimulating polypeptide may comprise one or more costimulatory signaling regions such as CD27, ICOS, RANK, TRANCE, CD28, 4-1BB, OX40, DAP10, MyD88, or CD40 or, for example, the cytoplasmic regions thereof. An inducible chimeric stimulating polypeptide may comprise one or more suitable costimulatory signaling regions that activate the signaling pathways activated by CD27, ICOS, RANK, TRANCE, CD28, 4-1BB, OX40, DAP10, MyD88, or CD40. Inducible chimeric stimulating polypeptides may include any polypeptide that activates the NF-κB pathway, Akt pathway, and/or p38 pathway of tumor necrosis factor receptor (TNFR) family (i.e., CD40, RANK/TRANCE-R, OX40, 4-1BB) and CD28 family members (CD28, ICOS). More than one costimulating polypeptide or costimulating polypeptide cytoplasmic region may be expressed in the modified T cells discussed herein.

Costimulation Provided by MyD88 and CD40

In some embodiments, a CAR T cell population described herein comprise an inducible chimeric stimulating polypeptide. The inducible chimeric stimulating polypeptide can comprise one or more costimulatory signaling regions that activate the signaling pathways activated by CD27, ICOS, RANK, TRANCE, CD28, 4-1BB, OX40, DAP10, MyD88, or CD40.

One of the principal functions of second generation CARs is the ability to produce IL-2 that supports T cell survival and growth through activation of the nuclear factor of activated T cells (NFAT) transcription factor by CD3ζ (signal 1) and NF-κB (signal 2) by CD28 or 4-1BB.32. Other molecules that similarly activate NF-κB may also be paired with the CD3ζ chain within a CAR molecule. One approach employs a T cell costimulating molecule that was originally developed as an adjuvant for a dendritic cell (DC) vaccine (Narayanan et al. (2011) J Clin Invest 121:1524-1534; Kemnade et al. (2012) Mol Ther 20(7):1462-1471). For full activation or licensing of DCs, Toll-like receptor (TLR) signaling is usually involved. In TLR signaling, the cytoplasmic TLR/IL-1 domains (referred to as TIR domains) of TLRs dimerize which leads to recruitment and association of cytosolic adaptor proteins such as, for example, the myeloid differentiation primary response protein (MyD88; see SEQ ID NO: 29 or SEQ ID NO: 47 for full length amino acid sequence and SEQ ID NO: 30 or SEQ ID NO: 48 for a nucleotide sequence encoding it).

In some embodiments, the CAR T cell population described herein comprises an inducible chimeric stimulating polypeptide comprising one or more costimulatory signaling regions that activate the signaling pathways activated by MyD88, CD40 and/or a MyD88-CD40 fusion chimeric polypeptide.

MyD88 is a universal adaptor molecule for TLRs and a critical signaling component of the innate immune system, triggering an alert for foreign invaders, priming immune cell recruitment and activation. MyD88 is a cytosolic adapter protein that plays a central role in the innate and adaptive immune response. This protein functions as an essential signal transducer in the interleukin-1 and Toll-like receptor signaling pathways. These pathways regulate that activation of numerous proinflammatory genes. The encoded protein consists of an N-terminal death domain and a C-terminal Toll-interleukin1 receptor domain. MyD88 TIR domain is able to heterodimerize with TLRs and homodimerize with other MyD88 proteins. This in turn results in recruitment and activation of IRAK family kinases through interaction of the death domains (DD) at the amino terminus of MyD88 and IRAK kinases which thereby initiates a signaling pathway that leads to activation of JNK, p38 MAPK (mitogen-activated protein kinase) and NF-κB, a transcription factor that induces expression of cytokine- and chemokine-encoding genes (as well as other genes). MyD88 acts acts via IRAK1, IRAK2, IRF7 and TRAF6, leading to NF-κB activation, cytokine secretion and the inflammatory response. It also activates IRF1 resulting in its rapid migration into the nucleus to mediate an efficient induction of IFN-beta, NOS2/INOS, and IL12A genes. MyD88-mediated signaling in intestinal epithelial cells is crucial for maintenance of gut homeostasis and controls the expression of the antimicrobial lectin REG3G in the small intestine. TLR signaling also upregulates expression of CD40, a member of the tumor necrosis factor receptor (TNFR) family, which interacts with CD40 ligand (CD154 or CD40L) on antigen-primed CD4⁺ T cells.

CD40 is an important part of the adaptive immune response, aiding to activate APCs through engagement with its cognate CD40L, in turn polarizing a stronger CTL response. The CD40/CD154 signaling system is an important component in T cell function and B cell-T cell interactions. CD40 signaling proceeds through formation of CD40 homodimers and interactions with TNFR-associated factors (TRAFs), carried out by recruitment of TRAFs to the cytoplasmic domain of CD40, which leads to T cell activation involving several secondary signals such as the NF-κB, JNK and AKT pathways.

Apart from survival and growth advantages, MyD88 or MyD88-CD40 fusion chimeric polypeptide-based costimulation may also provide additional functions to CAR-modified T cells. MyD88 signaling is critical for both Th1 and Th17 responses and acts via IL-1 to render CD4⁺ T cells refractory to regulatory T cell (Treg)-driven inhibition (see, e.g., Schenten et al. (2014) Immunity 40:78-90). In addition, CD40 signaling in CD8⁺ T cells via Ras, P13K and protein kinase C, results in NF-κB-dependent induction of cytotoxic mediators granzyme and perforin that lyse CD4⁺CD25⁺ Treg cells (Martin et al. (2010) J Immunol 184:5510-5518). Thus, MyD88 and CD40 co-activation may render CAR-T cells resistant to the immunosuppressive effects of Treg cells, a function that could be critically important in the treatment of solid tumors and other types of cancers.

MyD88 and CD40 together in immune cells, including T cells, can act downstream on transcription factors to upregulate proinflammtory cytokines, Type I IFNs, and promote proliferation and survival. Along with signaling input from CD3ζ from a CAR, MyD88/CD40 makes for a potent and pleotropic costimulatory molecule. In some embodiments, the technology herein provides for CAR T cells comprising an inducible chimeric stimulating polypeptide comprising one or more costimulatory signaling regions that activate the signaling pathways activated by MyD88, CD40 and/or MyD88-CD40 fusion chimeric polypeptide. Examples of suitable costimulatory signaling regions include, but are not limited to, IRAK-4, IRAK-1, TRAF6, TRAF2, TRAF3, TRAF5, Act, JAK3, or any functional fragments thereof.

One approach to costimulation of CAR-T cells is to express a fusion protein (referred to as MC) of the signaling elements of MyD88. Survival and growth of such cells can be enhanced through activation of the NFAT transcription factor by CD3ζ, which is part of the chimeric antigen receptor (signal 1), and NF-κB (signal 2) by MyD88 and CD40. The activation of CAR-T cells expressing MC is observed with a cytoplasmic MyD88/CD40 chimeric fusion protein, lacking a membrane targeting region, and with a chimeric fusion protein comprising MyD88/CD40 and a membrane targeting region, such as, for example, a myristoylation region. CAR-T cells may co-express an inducible chimeric signaling polypeptide comprising a multimeric ligand binding region, such as, for example, FKBP12v36, and a MyD88 polypeptide or truncated MyD8 polypeptide, or a MyD88-CD40 or truncated MyD88-CD40 polypeptide (iMC). Cells that express both iMC and a first generation CAR allowed complete T cell activation that required both iMC and tumor recognition through the CAR, resulting in IL-2 production, CD25 receptor upregulation and T cell expansion, and the therapeutic efficacy was controlled by AP1903 in vivo. In some embodiments, CAR-T cells comprise a nucleic acid that encodes a first polynucleotide encoding the inducible chimeric signaling polypeptide and a second polynucleotide encoding the CAR. In some embodiments, the first polynucleotide is positioned 5′ of the second polynucleotide. In some embodiments, the first polynucleotide is positioned 3′ of the second polynucleotide. In some embodiments, a third polynucleotide encoding a linker polypeptide is positioned between the first and second polynucleotides. In some embodiments, the linker polypeptide is a 2A polypeptide, which may separate the polypeptides encoded by the first and second polynucleotides during, or after translation.

By MyD88, or MyD88 polypeptide, is meant the polypeptide product of the myeloid differentiation primary response gene 88, for example, but not limited to the human version, cited as NCBI Gene ID 4615. One example of a MyD88 polypeptide is presented as SEQ ID NO: 47. Another example of a MyD88 polypeptide is presented as SEQ ID NO: 29. By “truncated,” is meant that the protein is not full length and may lack, for example, a domain. For example, a truncated MyD88 is not full length and may, for example, be missing the TIR domain. In some embodiments, the truncated MyD88 polypeptide is encoded by the nucleic acid sequence of SEQ ID NO: 22, and comprises the amino acid sequence of SEQ ID NO: 21. By a nucleic acid sequence coding for “truncated MyD88” is meant the nucleic acid sequence coding for the truncated MyD88 peptide, the term may also refer to the nucleic acid sequence including the portion coding for any amino acids added as an artifact of cloning, including any amino acids coded for by the linkers. It is understood that where a method or construct refers to a truncated MyD88 polypeptide, the method may also be used, or the construct designed to refer to another MyD88 polypeptide, such as a full length MyD88 polypeptide. Where a method or construct refers to a full length MyD88 polypeptide, the method may also be used, or the construct designed to refer to a truncated MyD88 polypeptide. Functionally equivalent” or “a functional fragment” of a MyD88 polypeptide refers, for example, to a truncated MyD88 polypeptide whether lacking the TIR domain or not that is capable of amplifying the cell-mediated tumor killing response when expressed in cells, for example, T cells, NK cells, or NK-T cells, such as, for example, the T cell-mediated, NK cell-mediated, or NK-T cell-mediated response, by, for example, activating the NFκB pathway. Truncated MyD88 polypeptides may, for example, comprise amino acid residues 1-172 of the full length MyD88 amino acid sequence, for example, residues 1-172 of SEQ ID NO: 29 or SEQ ID NO: 47. In some embodiments, Truncated MyD88 polypeptides may, for example, comprise amino acid residues 1-151 or 1-155 of the full length MyD88 amino acid sequence, for example, residues 1-151 or 1-155 of SEQ ID NO: 29 or SEQ ID NO: 47. In some embodiments, truncated MyD88 polypeptides may, for example, comprise amino acid residues 1-152, 153, 154, 155, 156, 157, 158, 159, 160, 161, 162, 163, 164, 165, 166, 167, 168, 169, 170, or 171 of the full length MyD88 amino acid sequence; an example of a full length MyD88 amino acid sequence is provided as SEQ ID NO: 29 or SEQ ID NO: 47. In some embodiments, the truncated MyD88 amino acid sequence does not include contiguous amino acid residues 173-296 of the full length MyD88 amino acid sequence. In some embodiments, the truncated MyD88 amino acid sequence does not include contiguous amino acid residues 152-296 of the full length MyD88 amino acid sequence. In some embodiments, the truncated MyD88 amino acid sequence does not include contiguous amino acid residues 156-296 of the full length MyD88 amino acid sequence. In some embodiments, the truncated MyD88 amino acid sequence does not include contiguous amino acid residues 152, 153, 154, 155, 156, 157, 158, 159, 160, 161, 162, 163, 164, 165, 166, 167, 168, 169, 170, 171, or 172-296 of the full length MyD88 amino acid sequence. By “full length MyD88 amino acid sequence” is meant a full length MyD88 amino acid sequence that corresponds to, for example, SEQ ID NO: 29 or SEQ ID NO: 47. In the embodiments provided herein, a cytoplasmic CD40 polypeptide lacking the extracellular domain, may be located either upstream or downstream from the MyD88 or truncated MyD88 polypeptide portion.

The term “chimeric stimulating polypeptide” is interchangeable with “chimeric stimulating molecule,” “costimulatory polypeptide,” “costimulatory molecule,” “chimeric costimulating molecule,” and “chimeric costimulating polypeptide,” and grammatical variations thereof.

The terms “chimeric,” “fusion” and “chimeric fusion” are used interchangeably herein with reference to a polypeptide containing two or more proteins (or a portion(s) of one or more of the two or more proteins) that have been joined to create a chimeric polypeptide. The two or more proteins (or portions thereof) may be directly joined to each other, wherein a terminal amino acid residue of one protein (or portion thereof) is directly bonded to a terminal amino acid residue of another protein (or portion thereof), or may be joined through one or more intervening elements (e.g., one or more amino acids that are not part of either protein, such as a linker or adapter, or a non-amino acid polymer). For example, a polypeptide that is produced from nucleic acid encoding a fusion of a multimerizing protein (or portion thereof) and another protein (e.g., a DNA-binding protein, transcription activation protein, pro-apoptotic protein or protein component of an immune cell activation pathway), or portion thereof, may be referred to as a chimeric, fusion or chimeric fusion polypeptide.

Costimulation in T cells that express chimeric antigen receptors by MyD88 and CD40 polypeptides, and by chimeric signaling polypeptides comprising costimulatory polypeptide cytoplasmic signaling regions is discussed in U.S. Patent Application Publication No. US2016/0058857 and in International Patent Application Publication No. as WO2018/208849, each of which is incorporated by reference herein in its entirety for all purposes.

Non-limiting examples of chimeric polypeptides useful for inducing cell activation, and related methods for inducing CAR-T cell activation including, for example, expression constructs, methods for constructing vectors, and assays for activity or function, may also be found in the following patents and patent applications: U.S. Patent Application Publication No. US2014/0286987; International Patent Application Publication No. WO2014/151960; U.S. Patent Application Publication No. US2016/0046700; International Patent Application Publication No. WO2015/123527; U.S. Patent Application Publication No. US2004/0209836; U.S. Pat. No. 7,404,950; International Patent Application Publication No. WO2004/073641; U.S. Patent Application Publication No. US2011/0033388; U.S. Pat. No. 8,691,210; International Patent Application Publication No. WO2008/049113; U.S. Patent Application Publication No. US2014/0087468; U.S. Pat. No. 9,315,559; International Patent Application Publication No. WO2010/33949; U.S. Patent Application Publication No. US2011/0287038; International Patent Application Publication No. WO2011/130566; U.S. Patent Application Publication No. US2016/0175359; International Patent Application Publication No. WO2016/036746; International Patent Application Publication No. WO2016/100241; U.S. Patent Application Publication No. US2017/0166877; International Patent Application Publication No. WO2017/106185; and International Patent Application Publication No. WO2018/208849, each of which is incorporated by reference herein in its entirety, including all text, tables and drawings, for all purposes.

An inducible chimeric stimulating polypeptide (e.g., inducible MyD88/CD40 (iMC)) may comprise one or more ligand binding regions including, for example, one or more FKBP regions. In some embodiments, an inducible chimeric stimulating polypeptide (e.g., inducible MyD88/CD40 (iMC)) comprises one or more FKBP12 polypeptides. In some embodiments, an inducible chimeric stimulating polypeptide (e.g., inducible MyD88/CD40 (iMC)) comprises one or more suitable FKBP12 variant polypeptides, including variant polypeptides that bind to AP1903, or other synthetic homodimerizers such as, for example, AP20187 or AP2015. Variant polypeptides may include, for example, an FKBP region that has an amino acid substitution at position 36 selected from the group consisting of valine, leucine, isoleuceine and alanine (Clackson T, et al. Proc Natl Acad Sci USA (1998) 95:10437-10442). In some embodiments, an inducible chimeric stimulating polypeptide (e.g., inducible MyD88/CD40 (iMC)) comprises one or more FKBP12v36 polypeptides. In some embodiments, an inducible chimeric stimulating polypeptide (e.g., inducible MyD88/CD40 (iMC)) comprises two FKBP12v36 polypeptides.

AP1903, also known as rimiducid, (CAS Index Name: 2-Piperidinecarboxylic acid, 1-[(2S)-1-oxo-2-(3,4,5-trimethoxyphenyl)butyl]-, 1,2-ethanediylbis[imino(2-oxo-2,1-ethanediyl)oxy-3,1-phenylene[(1R)-3-(3,4-dimethoxyphenyl)propylidene]]ester, [2S-[1(R*),2R*[S*[S*[1(R*),2]]]]]-(9Cl) CAS Registry Number: 195514-63-7; Molecular Formula: C78H98N4O20 Molecular Weight: 1411.65), is a synthetic molecule that has proven safe in healthy volunteers (luliucci J D, et al. (2001) J Clin Pharmacol. 41:870-879).

As used herein, the term “pharmaceutically or pharmacologically acceptable” refers to molecular entities and compositions that do not produce adverse, allergic, or other untoward reactions when administered to an animal or a human.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells presented herein, its use in therapeutic compositions is contemplated. Supplementary active ingredients also can be incorporated into the compositions. In some embodiments, the subject is a mammal.

Engineering Expression Constructs

Expression constructs that express the present chimeric antigen receptors and inducible chimeric stimulating polypeptides are provided herein.

As used herein, the term “cDNA” is intended to refer to DNA prepared using messenger RNA (mRNA) as template. The advantage of using a cDNA, as opposed to genomic DNA or DNA polymerized from a genomic, non- or partially processed RNA template, is that the cDNA primarily contains coding sequences of the corresponding protein. There are times when the full or partial genomic sequence is used, such as where the non-coding regions are required for optimal expression or where non-coding regions such as introns are to be targeted in an antisense strategy.

As used herein, the term “polypeptide” is defined as a chain of amino acid residues, usually having a defined sequence. As used herein the term polypeptide may be interchangeable with the term “proteins”.

As used herein, the term “expression construct” or “transgene” is defined as any type of genetic construct containing a nucleic acid coding for gene products in which part or all of the nucleic acid encoding sequence is capable of being transcribed can be inserted into the vector. The transcript is translated into a protein, but it need not be. In certain embodiments, expression includes both transcription of a gene and translation of mRNA into a gene product. In other embodiments, expression only includes transcription of the nucleic acid encoding genes of interest. The term “therapeutic construct” may also be used to refer to the expression construct or transgene. The expression construct or transgene may be used, for example, as a therapy to treat hyperproliferative diseases or disorders, such as cancer, thus the expression construct or transgene is a therapeutic construct or a prophylactic construct. As used herein with reference to a disease, disorder or condition, the terms “treatment”, “treat”, “treated”, or “treating” refer to prophylaxis and/or therapy.

As used herein, the term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well and are discussed infra.

In certain examples, a polynucleotide coding for the chimeric antigen receptor, is included in the same vector, such as, for example, a viral or plasmid vector, as a polynucleotide coding for a second polypeptide. This second polypeptide may be, for example, a chimeric signaling polypeptide, an inducible costimulatory polypeptide, an inducible chimeric stimulating polypeptide, as discussed herein, or a marker polypeptide. In these examples, the construct may be designed with one promoter operably linked to a nucleic acid comprising a polynucleotide coding for the two polypeptides, linked by a 2A polypeptide. In this example, the first and second polypeptides are separated during translation, resulting in two polypeptides, or, in examples including a leaky 2A, either one, or two polypeptides. In other examples, the two polypeptides may be expressed separately from the same vector, where each nucleic acid comprising a polynucleotide coding for one of the polypeptides is operably linked to a separate promoter. In yet other examples, one promoter may be operably linked to the two polynucleotides, directing the production of two separate RNA transcripts, and thus two polypeptides; in one example, the promoter may be bi-directional, and the coding regions may be in opposite directions 5′-3′. Therefore, the expression constructs discussed herein may comprise at least one, or at least two promoters.

In some embodiments, a nucleic acid construct is contained within a viral vector. In certain embodiments, the viral vector is a retroviral vector. In certain embodiments, the viral vector is an adenoviral vector or a lentiviral vector. It is understood that in some embodiments, a cell is contacted with the viral vector ex vivo, and in some embodiments, the cell is contacted with the viral vector in vivo. Thus, an expression construct may be inserted into a vector, for example a viral vector or plasmid. The steps of the methods provided may be performed using any suitable method; these methods include, without limitation, methods of transducing, transforming, or otherwise providing nucleic acid to the cell, described herein.

As used herein, the term “gene” is defined as a functional protein-, polypeptide-, or peptide-encoding unit. As will be understood, this functional term includes genomic sequences, cDNA sequences, and smaller engineered gene segments that express, or are adapted to express, proteins, polypeptides, domains, peptides, fusion proteins and/or mutants.

As used herein, the term “polynucleotide” is defined as a chain of nucleotides. Furthermore, nucleic acids are polymers of nucleotides. Thus, nucleic acids and polynucleotides as used herein are interchangeable. Nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PORT™, and the like, and by synthetic means. Furthermore, polynucleotides include mutations of the polynucleotides, include but are not limited to, mutation of the nucleotides, or nucleosides by methods well known in the art. A nucleic acid may comprise one or more polynucleotides.

“Function-conservative variants” are proteins or enzymes in which a given amino acid residue has been changed without altering overall conformation and function of the protein or enzyme, including, but not limited to, replacement of an amino acid with one having similar properties, including polar or non-polar character, size, shape and charge. Conservative amino acid substitutions for many of the commonly known non-genetically encoded amino acids are well known in the art. Conservative substitutions for other non-encoded amino acids can be determined based on their physical properties as compared to the properties of the genetically encoded amino acids.

Amino acids other than those indicated as conserved may differ in a protein or enzyme so that the percent protein or amino acid sequence similarity between any two proteins of similar function may vary and can be, for example, at least 70%, at least 80%, at least 90%, and at least 95%, as determined according to an alignment scheme. As referred to herein, “sequence similarity” means the extent to which nucleotide or protein sequences are related. The extent of similarity between two sequences can be based on percent sequence identity and/or conservation. “Sequence identity” herein means the extent to which two nucleotide or amino acid sequences are invariant. “Sequence alignment” means the process of lining up two or more sequences to achieve maximal levels of identity (and, in the case of amino acid sequences, conservation) for the purpose of assessing the degree of similarity. Numerous methods for aligning sequences and assessing similarity/identity are known in the art such as, for example, the Cluster Method, wherein similarity is based on the MEGALIGN algorithm, as well as BLASTN, BLASTP, and FASTA. When using any of these programs, the settings may be selected that result in the highest sequence similarity.

As used herein, the term “promoter” is defined as a DNA sequence recognized by the synthetic machinery of the cell, or introduced synthetic machinery, required to initiate the specific transcription of a gene. In some embodiments, the promoter is a developmentally regulated promoter. As used herein, the term “under transcriptional control,” “operably linked,” or “operatively linked” is defined as the promoter is in the correct location and orientation in relation to the nucleic acid to control RNA polymerase initiation and expression of the gene. In some examples, one or more polypeptides are said to be “operatively linked.” In general, the term “operably linked” is meant to indicate that the promoter sequence is functionally linked to a second sequence, wherein the promoter sequence initiates and mediates transcription of the DNA corresponding to the second sequence.

The particular promoter employed to control the expression of a polynucleotide sequence of interest is not believed to be important, so long as it is capable of directing the expression of the polynucleotide in the targeted cell. Thus, where a human cell is targeted the polynucleotide sequence-coding region may, for example, be placed adjacent to and under the control of a promoter that is capable of being expressed in a human cell. Generally speaking, such a promoter might include either a human or viral promoter. Promoters may be selected that are appropriate for the vector used to express the CARs and other polypeptides provided herein.

In various embodiments, where, for example, the expression vector is a retrovirus, an example of an appropriate promoter is the Murine Moloney leukemia virus promoter. In other embodiments, the promoter may be, for example, may be the (CMV) immediate early gene promoter, the SV40 early promoter, the Rous sarcoma virus long terminal repeat, ß-actin, rat insulin promoter and glyceraldehyde-3-phosphate dehydrogenase can be used to obtain high-level expression of the coding sequence of interest. The use of other viral or mammalian cellular or bacterial phage promoters which are well known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized.

Promoters, and other regulatory elements, are selected such that they are functional in the desired cells or tissue. In addition, this list of promoters should not be construed to be exhaustive or limiting; other promoters that are used in conjunction with the promoters and methods disclosed herein.

The nucleic acids discussed herein may comprise one or more polynucleotides. In some embodiments, one or more polynucleotides may be described as being positioned, or “is” “5′” or “3′” of another polynucleotide, or positioned in “5′ to 3′ order”. The reference 5′ to 3′ in these contexts is understood to refer to the direction of the coding regions of the polynucleotides in the nucleic acid, for example, where a first polynucleotide is positioned 5′ of a second polynucleotide and connected with a third polynucleotide encoding a non-cleave able linker polypeptide, the translation product would result in the polypeptide encoded by the first polynucleotide positioned at the amino terminal end of a larger polypeptide comprising the translation products of the first, third, and second polynucleotides.

In some embodiments, two or more polypeptides, such as, for example, a chimeric antigen receptor, an inducible chimeric stimulating polypeptide (e.g., inducible MyD88/CD40 (iMC)), and/or a further polypeptide, may be expressed in a cell using two separate vectors. The cells may be co-transfected or co-transformed with the vectors, or the vectors may be introduced to the cells at different times.

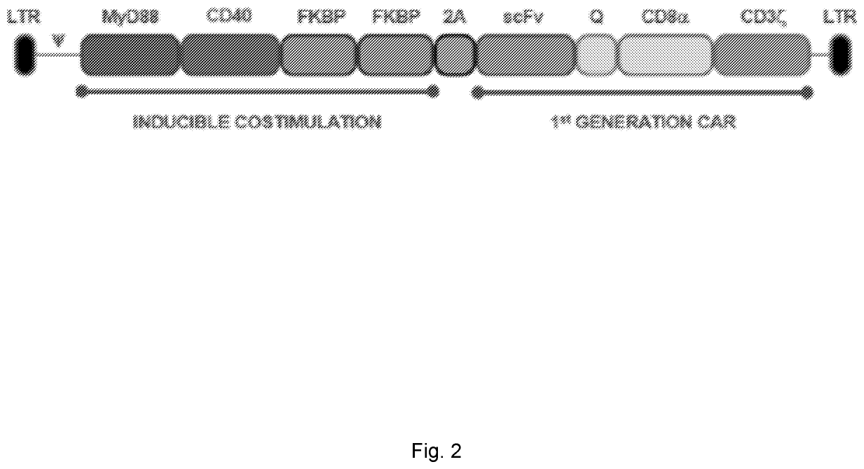

The polypeptides may vary in their order, from the amino terminus to the carboxy terminus. For example, the order of the vector components presented in FIG. 2 and discussed in Example 1 may vary. In some embodiments, the order of the components of the inducible chimeric stimulating polypeptide may vary. In some embodiments, the order of the components of the chimeric antigen receptor polypeptide may vary. The order of the various domains may be assayed using methods such as, for example, those discussed herein, to obtain the optimal expression and activity.

In some embodiments, where an expression construct encodes a MyD88 polypeptide, the polypeptide may be a portion of the full-length MyD88 polypeptide. By MyD88, or MyD88 polypeptide, is meant the polypeptide product of the myeloid differentiation primary response gene 88, for example, but not limited to the human version, cited as NCBI Gene ID 4615. In some embodiments, an expression construct encodes a portion of the MyD88 polypeptide lacking the TIR domain. In some embodiments, the expression construct encodes a portion of the MyD88 polypeptide containing the DD (death domain) or the DD and intermediary domains. By “truncated,” is meant that the protein is not full length and may lack, for example, a domain. For example, a truncated MyD88 is not full length and may, for example, be missing the TIR domain. In some embodiments, the truncated MyD88 polypeptide has an amino acid sequence of SEQ ID NO: 21, or a functionally equivalent fragment thereof. In some embodiments, the truncated MyD88 polypeptide is encoded by the nucleotide sequences of SEQ ID NO: 22, or a functionally equivalent fragment thereof. A functionally equivalent portion of the MyD88 polypeptide has substantially the same ability to stimulate intracellular signaling as the polypeptide of SEQ ID NO: 21, with at least 50%, 60%, 70%, 80%, 90%, or 95% of the activity of the polypeptide of SEQ ID NO: 21. In some embodiments, the expression construct encodes a portion of a MyD88 polypeptide lacking the TIR domain. By a nucleic acid sequence coding for “truncated MyD88” is meant the nucleic acid sequence coding for a truncated MyD88 polypeptide, the term may also refer to the nucleic acid sequence including the portion coding for any amino acids added as an artifact of cloning, including any amino acids coded for by the linkers.

It is understood that where a method or construct refers to a truncated MyD88 polypeptide, the method may also be used, or the construct designed to refer to another MyD88 polypeptide, such as a full length MyD88 polypeptide. Where a method or construct refers to a full length MyD88 polypeptide, the method may also be used, or the construct designed to refer to a truncated MyD88 polypeptide. In the methods herein, in which a chimeric polypeptide comprises a MyD88 polypeptide (or portion thereof) and a CD40 polypeptide (or portion thereof), the MyD88 polypeptide of the chimeric polypeptide may be located either upstream or downstream from the CD40 polypeptide. In certain embodiments, the MyD88 polypeptide (or portion thereof) is located upstream of the CD40 polypeptide (or portion thereof). As used herein, the term “functionally equivalent,” as it relates to MyD88, or a portion thereof, for example, refers to a MyD88 polypeptide that stimulates a cell-signaling response or a nucleic acid encoding such a MyD88 polypeptide. “Functionally equivalent” refers, for example, to a MyD88 polypeptide that is lacking a TIR domain but is capable of stimulating a cell-signaling response.

Selectable Markers

In certain embodiments, the expression constructs contain nucleic acid constructs whose expression is identified in vitro or in vivo by including a marker in the expression construct. Such markers would confer an identifiable change to the cell permitting easy identification of cells containing the expression construct. Usually the inclusion of a drug selection marker aids in cloning and in the selection of transformants. For example, genes that confer resistance to neomycin, puromycin, hygromycin, DHFR, GPT, zeocin and histidinol are useful selectable markers. Alternatively, enzymes such as Herpes Simplex Virus thymidine kinase (tk) are employed. Immunologic surface markers containing the extracellular, non-signaling domains or various proteins (e.g. CD34, CD19, LNGFR) also can be employed, permitting a straightforward method for magnetic or fluorescence antibody-mediated sorting. The selectable marker employed is not believed to be important, so long as it is capable of being expressed simultaneously with the nucleic acid encoding a gene product. Further examples of selectable markers include, for example, reporters such as GFP, EGFP, β-gal or chloramphenicol acetyltransferase (CAT). In certain embodiments, the marker protein, such as, for example, CD19 is used for selection of the cells for transfusion, such as, for example, in immunomagnetic selection. As discussed herein, a CD19 marker is distinguished from an anti-CD19 antibody, or, for example, a scFv, TCR, or other antigen recognition moiety that binds to CD19.

In certain embodiments, a marker polypeptide is linked to an inducible chimeric stimulating polypeptide. For example, a marker polypeptide may be linked to an inducible chimeric stimulating polypeptide via a polypeptide sequence, such as, for example, a cleavable 2A-like sequence.

The CAR-T cells provided herein may express a cell surface transgene marker, present on an expression vector that expresses the CAR, or, in some embodiments, present on an expression vector that encodes a protein other than the CAR, such as, for example an inducible chimeric stimulating polypeptide, that is co-expressed with the CAR.

In one embodiment, the cell surface transgene marker is a truncated CD19 (ΔCD19) polypeptide (Di Stasi et al. (2011) supra), that comprises a human CD19 truncated at amino acid 333 to remove most of the intracytoplasmic domain. The extracellular CD19 domain can still be recognised (e.g. in flow cytometry, FACS or MACS) but the potential to trigger intracellular signalling is minimised. CD19 is normally expressed by B cells, rather than by T cells, so selection of CD19+ T cells permits the genetically-modified T cells to be separated from unmodified donor T cells.

In some embodiments, a marker polypeptide may be included in the polypeptide, for example, the CAR encoded by the expression vector to aid in sorting cells. In some embodiments, the expression vectors used to express the chimeric antigen receptors or inducible chimeric stimulating polypeptides provided herein further comprise a polynucleotide that encodes the 16 amino acid CD34 minimal epitope. In some embodiments, the CD34 minimal epitope is incorporated at the amino terminal position of the CD8 stalk.

Linker Polypeptides

Linker polypeptides include, for example, cleavable and non-cleavable linker polypeptides. Non-cleavable polypeptides may include, for example, any polypeptide that may be operably linked between the MyD88-CD40 chimeric polypeptide, the MyD88 polypeptide, the CD40 polypeptide, or the costimulatory polypeptide cytoplasmic signaling region and the CD3ζ portion of the chimeric antigen receptor. Linker polypeptides include those for example, consisting of about 2 to about 30 amino acids, (e.g., furin cleavage site, (GGGGS)ₙ). In some embodiments, the linker polypeptide consists of about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids. In some embodiments, the linker polypeptide consists of about 18 to 22 amino acids. In some embodiments, the linker polypeptide consists of 20 amino acids. In some embodiments, cleavable linkers include linkers that are cleaved by an enzyme exogenous to the modified cells in the population, for example, an enzyme encoded by a polynucleotide that is introduced into the cells by transfection or transduction, either at the same time or a different time as the polynucleotide that encodes the linker. In some embodiments, cleavable linkers include linkers that are cleaved by an enzyme endogenous to the modified cells in the population, including, for example, enzymes that are naturally expressed in the cell, and enzymes encoded by polynucleotides native to the cell, such as, for example, lysozyme.

2A Peptide Bond-Skipping Sequences

2A-like sequences, or “peptide bond-skipping” 2A sequences, are derived from, for example, many different viruses, including, for example, from Thosea asigna. These sequences are sometimes also known as “peptide skipping sequences.” When this type of sequence is placed within a cistron, between two polypeptides that are intended to be separated, the ribosome appears to skip a peptide bond, in the case of Thosea asigna sequence; the bond between the Gly and Pro amino acids at the carboxy terminal “P-G-P” is omitted. This may, leave two to three polypeptides, for example, an inducible chimeric stimulating polypeptide and a chimeric antigen receptor, or, for example, a marker polypeptide and an inducible chimeric stimulating polypeptide. When this sequence is used, the polypeptide that is encoded 5′ of the 2A sequence may end up with additional amino acids at the carboxy terminus, including the Gly residue and any upstream residues in the 2A sequence. The peptide that is encoded 3′ of the 2A sequence may end up with additional amino acids at the amino terminus, including the Pro residue and any downstream residues following the 2A sequence. In some embodiments, the cleavable linker is a 2A polypeptide derived from porcine teschovirus-1 (P2A). In some embodiments, the 2A cotranslational sequence is a 2A-like sequence. In some embodiments, the 2A cotranslational sequence is T2A (thosea asigna virus 2A), F2A (foot and mouth disease virus 2A), P2A (porcine teschovirus-1 2A), BmCPV 2A (cytoplasmic polyhedrosis virus 2A) BmIFV 2A (flacherie virus of B. mori 2A), or E2A (equine rhinitis A virus 2A). In some embodiments, the 2A cotranslational sequence is T2A-GSG, F2A-GSG, P2A-GSG, or E2A-GSG. In some embodiments, the 2A cotranslational sequence is selected from the group consisting of T2A, P2A and F2A. By “cleavable linker” is meant that the linker is cleaved by any means, including, for example, non-enzymatic means, such as peptide skipping, or enzymatic means. (Donnelly, M L 2001, J. Gen. Virol. 82:1013-25).

The 2A-like sequences are sometimes “leaky” in that some of the polypeptides are not separated during translation, and instead, remain as one long polypeptide following translation. One theory as to the cause of the leaky linker, is that the short 2A sequence occasionally may not fold into the required structure that promotes ribosome skipping (a “2A fold”). In these instances, ribosomes may not miss the proline peptide bond, which then results in a fusion protein. To reduce the level of leakiness, and thus reduce the number of fusion proteins that form, a GSG (or similar) linker may be added to the amino terminal side of the 2A polypeptide; the GSG linker blocks secondary structures of newly-translated polypeptides from spontaneously folding and disrupting the ‘2A fold’.

In certain embodiments, a 2A linker includes the amino acid sequence of SEQ ID NO: 19. In certain embodiments, the 2A linker further includes a GSG amino acid sequence at the amino terminus of the polypeptide, in other embodiments, the 2A linker includes a GSGPR amino acid sequence at the amino terminus of the polypeptide. Thus, by a “2A” sequence, the term may refer to a 2A sequence in an example described herein or may also refer to a 2A sequence as listed herein further comprising a GSG or GSGPR sequence at the amino terminus of the linker.

In some embodiments, the linker, for example, the 2A linker, is cleaved in about 10, 20, 30, 40, 50, 60, 70, 75, 80, 85, 90, 95, 98, or 99% of the chimeric antigen receptors, that is, the chimeric antigen receptor portion is separated from the chimeric MyD88 and CD40, the MyD88 polypeptide, the CD40 polypeptide, or the costimulatory polypeptide cytoplasmic signaling region, such as, CD28, OX40, 4-1BB or the like. In other embodiments the 2A linker is cleaved in about 75, 80, 85, 90, 95, 98, or 99% of the chimeric antigen receptors. In some embodiments, the 2A linker is cleaved in about 80-99% of the chimeric antigen receptors. In some embodiments, the 2A linker is cleaved in about 90% of the chimeric antigen receptors. In some embodiments, a constitutive active chimeric antigen receptor polypeptide is present in the modified cells, where the 2A linker is not cleaved, that is, the chimeric antigen receptor portion is linked to the chimeric MyD88 and CD40, the MyD88 polypeptide, the CD40 polypeptide, or the costimulatory polypeptide cytoplasmic signaling region, such as, CD28, OX40, 4-1BB or the like, representing about 1, 2, 5, 10, 15, 20, 25, 30, 40, 50, 60, 70, 80, or 90% of the chimeric antigen receptor polypeptide. In other embodiments the 2A linker is not cleaved in about 5, 10, 15, 20, or 25% of the chimeric antigen receptors. In some embodiments, the 2A linker is not cleaved in about 5-20% of the chimeric antigen receptors. In some embodiments, the 2A linker is not cleaved in about 10% of the chimeric antigen receptors.

Chimeric Antigen Receptors

Antigen Recognition Moieties

An “antigen recognition moiety” may be any polypeptide or fragment thereof, such as, for example, an antibody fragment variable domain, either naturally derived, or synthetic, which binds to an antigen. Examples of antigen recognition moieties include, but are not limited to, polypeptides derived from antibodies, such as, for example, single chain variable fragments (scFv), Fab, Fab′, F(ab′)₂, and Fv fragments; polypeptides derived from T Cell receptors, such as, for example, TCR variable domains; secreted factors (e.g., cytokines, growth factors) that can be artificially fused to signaling domains (e.g., “zytokines”), and any ligand or receptor fragment (e.g., CD27, NKG2D) that binds to the extracellular cognate protein. Combinatorial libraries could also be used to identify peptides binding with high affinity to tumor-associated targets. Moreover, “universal” CARs can be made by fusing aviden to the signaling domains in combination with biotinylated tumor-targeting antibodies or by using Fc gamma receptor/CD16 to bind to IgG-targeted tumors.

Transmembrane Regions

A chimeric protein herein may include a single-pass or multiple pass transmembrane sequence (e.g., at the N-terminus or C-terminus of the chimeric protein). Single pass transmembrane regions are found in certain CD molecules, tyrosine kinase receptors, serine/threonine kinase receptors, TGFβ, BMP, activin and phosphatases. Single pass transmembrane regions often include a signal peptide region and a transmembrane region of about 20 to about 25 amino acids, many of which are hydrophobic amino acids and can form an alpha helix. A short track of positively charged amino acids often follows the transmembrane span to anchor the protein in the membrane. Multiple pass proteins include ion pumps, ion channels, and transporters, and include two or more helices that span the membrane multiple times. All or substantially all of a multiple pass protein sometimes is incorporated in a chimeric protein. Sequences for single pass and multiple pass transmembrane regions are known and can be selected for incorporation into a chimeric protein molecule.

In some embodiments, the transmembrane domain is fused to the extracellular domain of a CAR. In one embodiment, the transmembrane domain that naturally is associated with one of the domains in a CAR is used. In other embodiments, a transmembrane domain that is not naturally associated with one of the domains in the CAR is used. In some instances, the transmembrane domain can be selected or modified by amino acid substitution (e.g., typically charged to a hydrophobic residue) to avoid binding of such domains to the transmembrane domains of the same or different surface membrane proteins to minimize interactions with other members of the receptor complex.

Transmembrane domains may, for example, be derived from the alpha, beta, or zeta chain of the T cell receptor, CD3-ε, CD3 ζ, CD4, CD5, CD8, CD8α, CD9, CD16, CD22, CD28, CD33, CD38, CD64, CD80, CD86, CD134, CD137, or CD154. Or, in some examples, the transmembrane domain may be synthesized de novo, comprising mostly hydrophobic residues, such as, for example, leucine and valine. In certain embodiments a short polypeptide linker may form the linkage between the transmembrane domain and the intracellular domain of a chimeric antigen receptor. A chimeric antigen receptors may further comprise a stalk, that is, an extracellular region of amino acids between the extracellular domain and the transmembrane domain. For example, the stalk may be a sequence of amino acids naturally associated with the selected transmembrane domain. In some embodiments, the chimeric antigen receptor comprises a CD8 transmembrane domain, in certain embodiments, the chimeric antigen receptor comprises a CD8 transmembrane domain, and additional amino acids on the extracellular portion of the transmembrane domain, in certain embodiments, the chimeric antigen receptor comprises a CD8 transmembrane domain and a CD8 stalk. A chimeric antigen receptor may further comprise a region of amino acids between the transmembrane domain and the cytoplasmic domain, which are naturally associated with the polypeptide from which the transmembrane domain is derived.

In some embodiments, a chimeric antigen receptor is an anti-PSCA chimeric antigen receptor. An anti-PSCA chimeric antigen receptor comprises an antigen recognition moiety that binds to PSCA. Examples of antigen recognition moiety polypeptides, and variable heavy and light chain amino acid sequences that bind to PSCA are provided in, for example, U.S. Pat. No. 8,013,128, by Gudas, J., issued Sep. 6, 2011, published as US 2009-0181034 A1 on Jul. 16, 2009; U.S. Pat. No. 8,206,932, by Gudas, J., issued Jun. 26, 2012, published as US 2009-0202548 A1 on Aug. 13, 2009; U.S. Pat. No. 7,541,442, issued Jun. 2, 2009, published as US 2006-0147375 A1 on Jul. 6, 2006; and U.S. Pat. No. 8,940,298, by Wu, A. W., et al., issued on Jan. 27, 2015, published as US 2010-0297004 on Nov. 25, 2010, which are all hereby incorporated by reference in their entireties. Examples of antigen recognition moiety polypeptides may be derived from a PSCA antibody selected from the group consisting of 1G8, 2A2, 2H9, 3C5, 3E6, 3G3 and 4A10 produced by the hybridomas designated HB-12612, HB-12613, HB-12614, HB-12616, HB-12618, HB-12615, and HB-12617, respectively, as deposited with the American Type Culture Collection, and those discussed in, for example, U.S. Pat. No. 7,527,786, by Reiter, R. E., et al., issued May 5, 2009, published as US 2003-0113810 A1 on Jun. 19, 2003, which are all hereby incorporated by reference in their entireties. By “derived from” is meant that antigen recognition moiety comprises polypeptides comprising the variable heavy chain polypeptide and the variable light chain polypeptide of the PSCA antibody produced by the designated hybridoma, or polypeptides having 70, 80, 90, 95, 97, 98, or 99 percent homology thereto, where the antigen recognition moiety binds to PSCA.

In some embodiments, the antigen recognition moiety polypeptide is a single-chain variable region domain (scFV) from a PSCA antibody selected from the group consisting of 1G8, 2A2, 2H9, 3C5, 3E6, 3G3 and 4A10 produced by the hybridomas designated HB-12612, HB-12613, HB-12614, HB-12616, HB-12618, HB-12615, and HB-12617, respectively, as deposited with the American Type Culture Collection, and those discussed in, for example, U.S. Pat. No. 7,527,786, by Reiter, R. E., et al., issued May 5, 2009, published as US 2003-0113810 A1 on Jun. 19, 2003, which are all hereby incorporated by reference in their entireties. In some embodiments, the single-chain variable region domain (scFV) is derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, PSCA-specific CAR-T cells target PSCA with a first-generation CAR construct containing a traditional CD3ζ cytoplasmic signaling domain together with a single-chain variable region domain (scFV) containing the variable heavy chain and variable light chain A11 VL (SEQ ID NO: 49), and A11 VH (SEQ ID NO: 51), respectively. In some embodiments, the anti-PSCA CAR containing the variable heavy chain and variable light chain A11 VL (SEQ ID NO: 49), and A11 VH (SEQ ID NO: 51) is engineered to express an inducible dual co-stimulatory domain comprised of two FK binding proteins (FKBP) in-frame with the signaling domains from MyD88 and CD40 (inducible MyD88/CD40 or iMC).

In some embodiments, PSCA-specific CAR-T cells target PSCA with a first-generation CAR construct containing a traditional CD3ζ cytoplasmic signaling domain together with a single-chain variable region domain (scFV) containing the variable heavy chain and variable light chain bm2B3 VL (SEQ ID NO:53) and bm2B3 VH (SEQ ID NO:55), respectively. In some embodiments, the anti-PSCA CAR containing the variable heavy chain and variable light chain bm2B3 VL (SEQ ID NO:53) and bm2B3 VH (SEQ ID NO:55) is engineered to express an inducible dual co-stimulatory domain comprised of two FK binding proteins (FKBP) in-frame with the signaling domains from MyD88 and CD40 (inducible MyD88/CD40 or iMC).

In some embodiments, PSCA-specific CAR-T cells target PSCA with a first-generation CAR construct containing a traditional CD3ζ cytoplasmic signaling domain together with a single-chain variable region domain (scFV) containing the variable heavy chain and variable light chain HA1-4.121 VH (SEQ ID NO: 57) and HLA1-4.121 VL (SEQ ID NO: 59), respectively. In some embodiments, the anti-PSCA CAR containing the variable heavy chain and variable light chain HA1-4.121 VH (SEQ ID NO: 57) and HLA1-4.121 VL (SEQ ID NO: 59) is engineered to express an inducible dual co-stimulatory domain comprised of two FK binding proteins (FKBP) in-frame with the signaling domains from MyD88 and CD40 (inducible MyD88/CD40 or iMC).

In some embodiments, PSCA-specific CAR-T cells target PSCA with a first-generation CAR construct containing a traditional CD3ζ cytoplasmic signaling domain together with a single-chain variable region domain (scFV) containing the variable heavy chain and variable light chain H1-1.10 VH (SEQ ID NO: 61) and H1-1.10 VL (SEQ ID NO: 63), respectively. In some embodiments, the anti-PSCA CAR containing the variable heavy chain and variable light chain H1-1.10 VH (SEQ ID NO: 61) and H1-1.10 VL (SEQ ID NO: 63) is engineered to express an inducible dual co-stimulatory domain comprised of two FK binding proteins (FKBP) in-frame with the signaling domains from MyD88 and CD40 (inducible MyD88/CD40 or iMC).

In some embodiments, PSCA-specific CAR-T cells target PSCA with a first-generation CAR construct containing a traditional CD3ζ cytoplasmic signaling domain together with a single-chain variable region domain (scFV) containing the variable heavy chain and variable light chain 1G8 (2B3) (SEQ ID NO: 65) and 1G8 (2B3-05) (SEQ ID NO: 67), respectively. In some embodiments, the anti-PSCA CAR containing the variable heavy chain and variable light chain 1G8 (2B3) (SEQ ID NO: 65) and 1G8 (2B3-05) (SEQ ID NO: 67) is engineered to express an inducible dual co-stimulatory domain comprised of two FK binding proteins (FKBP) in-frame with the signaling domains from MyD88 and CD40 (inducible MyD88/CD40 or iMC).

In some embodiments, PSCA-specific CAR-T cells target PSCA with a first-generation CAR construct containing a traditional CD3ζ cytoplasmic signaling domain together with a single-chain variable region domain (scFV) containing the variable heavy chain and variable light chain 1G8 (2B3-A2) (SEQ ID NO: 69) and 1G8 (2B3-A11) (SEQ ID NO: 71), respectively. In some embodiments, the anti-PSCA CAR containing the variable heavy chain and variable light chain 1G8 (2B3-A2) (SEQ ID NO: 69) and 1G8 (2B3-A11) (SEQ ID NO: 71) is engineered to express an inducible dual co-stimulatory domain comprised of two FK binding proteins (FKBP) in-frame with the signaling domains from MyD88 and CD40 (inducible MyD88/CD40 or iMC).

Methods of Gene Transfer/Genetic Modification of T Cells

In order to mediate the effect of the transgene expression in a cell, it will be necessary to transfer the expression constructs into a cell. Such transfer may employ viral or non-viral methods of gene transfer. This section provides a discussion of methods and compositions of gene transfer.

A transformed cell comprising an expression vector is generated by introducing into the cell the expression vector. Suitable methods for polynucleotide delivery for transformation of an organelle, a cell, a tissue or an organism for use with the current methods include virtually any method by which a polynucleotide (e.g., DNA) can be introduced into an organelle, a cell, a tissue or an organism.

The terms “cell,” “cell line,” and “cell culture” as used herein may be used interchangeably. All of these terms also include their progeny, which are any and all subsequent generations. It is understood that all progeny may not be identical due to deliberate or inadvertent mutations. As used herein, the term “ex vivo” refers to “outside” the body. The terms “ex vivo” and “in vitro” can be used interchangeably herein.

The term “transfection” and “transduction” are interchangeable and refer to the process by which an exogenous nucleic acid sequence is introduced into a eukaryotic host cell. Transfection (or transduction) can be achieved by any one of a number of means including electroporation, microinjection, gene gun delivery, retroviral infection, lipofection, superfection and the like.

Any appropriate method may be used to transfect or transform the cells, for example, the T cells, or to administer the nucleotide sequences or compositions of the present methods. Certain non-limiting examples are presented herein. In some embodiments, the viral vector is an SFG-based viral vector, as discussed in Tey et al. (2007) Biol Blood Marrow Transpl 13:913-24 and by Di Stasi et al. (2011) N Engl J Med 365:1673-83 (2011).

T cells that are genetically modified as disclosed herein are useful for administering to subjects who can benefit from donor lymphocyte administration. These subjects will typically be humans, so the methods herein will typically be performed using human T cells.

The modified cells may be obtained from a donor, or may be cells obtained from the patient, for example, the cells may be autologous, syngeneic, or allogeneic. The cells may, for example, be used in regeneration, for example, to replace the function of diseased cells. The cells may also be modified to express a heterologous gene so that biological agents may be delivered to specific microenvironments such as, for example, diseased bone marrow or metastatic deposits. By “therapeutic cell” is meant a cell used for cell therapy, that is, a cell administered to a subject to treat or prevent a condition or disease.

By “obtained or prepared” as, for example, in the case of cells, is meant that the cells or cell culture are isolated, purified, or partially purified from the source, where the source may be, for example, umbilical cord blood, bone marrow, or peripheral blood. The terms may also apply to the case where the original source, or a cell culture, has been cultured and the cells have replicated, and where the progeny cells are now derived from the original source.

Peripheral blood: The term “peripheral blood” as used herein, refers to cellular components of blood (e.g., red blood cells, white blood cells and platelets), which are obtained or prepared from the circulating pool of blood and not sequestered within the lymphatic system, spleen, liver or bone marrow.

Umbilical cord blood: Umbilical cord blood is distinct from peripheral blood and blood sequestered within the lymphatic system, spleen, liver or bone marrow. The terms “umbilical cord blood”, “umbilical blood” or “cord blood”, which can be used interchangeably, refers to blood that remains in the placenta and in the attached umbilical cord after child birth. Cord blood often contains stem cells including hematopoietic cells.

The term “allogeneic” as used herein, refers to HLA or MHC loci that are antigenically distinct between the host and donor cells. Thus, cells or tissue transferred from the same species can be antigenically distinct. Syngeneic mice can differ at one or more loci (congenics) and allogeneic mice can have the same background. The term “autologous” means a cell, nucleic acid, protein, polypeptide, or the like derived from the same individual to which it is later administered. The modified cells of the present methods may, for example, be autologous cells, such as, for example, autologous T cells.

Donor T cells are generally cultured (usually under activating conditions e.g. using anti-CD3 and/or anti-CD28 antibodies, optionally with IL-2) prior to being genetically modified. This step provides higher yields of T cells at the end of the modification process.

The sample may be subjected to allodepletion in some embodiments or may not be subjected to allodepletion. In examples provided herein, the samples are not subject to allodepletion, and are thus alloreplete, as discussed in Zhou et al. (2015) Blood 125:4103-13. These populations provide a more robust T cell repertoire for providing the therapeutic advantages of the donor cells.

The T cells can be transduced using a viral vector encoding polynucleotides of the present application. Suitable transduction techniques may involve fibronectin fragment CH-296. As an alternative to transduction using a viral vector, cells can be transfected with any suitable method known in the art such as with DNA encoding an inducible chimeric stimulating polypeptide and/or a chimeric antigen receptor of interest e.g. using calcium phosphate, cationic polymers (such as PEI), magnetic beads, electroporation and commercial lipid-based reagents such as Lipofectamine™ and Fugene™. One result of the transduction/transfection step is that various donor T cells will now be genetically-modified T cells which can express the polypeptide(s) of interest.

Viral vectors encoding the desired proteins may be used in certain embodiments. In some embodiments, retroviral vectors that can provide a high copy number of proviral integrants per cell are used for transduction.

After transduction/transfection, cells can be separated from transduction/transfection materials and cultured again, to permit the genetically-modified T cells to expand. T cells can be expanded so that a desired minimum number of genetically-modified T cells is achieved.

Genetically-modified T cells can then be selected from the population of cells which has been obtained. The inducible chimeric stimulating polypeptide and/or the chimeric antigen receptor may not be suitable for positive selection of desired T cells, so in some embodiments, the genetically-modified T cells express a cell surface transgene marker of interest. Cells which express this surface marker can be selected e.g. using immunomagnetic techniques. For instance, paramagnetic beads conjugated to monoclonal antibodies which recognise the cell surface transgene marker of interest can be used, for example, using a CliniMACS system (available from Miltenyi Biotec). In some embodiments, a selection step is not performed and a mixture or genetically modified T cells and unmodified T cells are administered to a subject.

In an alternative procedure, genetically-modified T cells are selected after a step of transduction, are cultured, and are then fed. Thus the order of transduction, feeding, and selection can be varied.

The result of these procedures is a composition containing donor T cells which have been genetically modified and which can thus express, e.g. an inducible chimeric stimulating polypeptide and/or a chimeric antigen receptor of interest (and, sometimes, a cell surface transgene marker of interest). These genetically-modified T cells can be administered to a recipient, and may be cryopreserved (optionally after further expansion) before being administered.

Methods of Treatment

Modified cell populations provided herein may be used in methods for treating human subjects in need thereof, and may be used to prepare medicaments for treating such subjects. The cells will usually be delivered to the recipient subject by infusion.

The term “terms “patient” or “subject” are interchangeable, and, as used herein include, but are not limited to, an organism or animal; a mammal, including, e.g., a human, non-human primate (e.g., monkey), mouse, pig, cow, goat, rabbit, rat, guinea pig, hamster, horse, monkey, sheep, or other non-human mammal; a non-mammal, including, e.g., a non-mammalian vertebrate, such as a bird (e.g., a chicken or duck) or a fish, and a non-mammalian invertebrate. The subject may be, for example, human, for example, a patient that is immunocompromised, or is suffering from a hyperproliferative disease. The subject may be, for example, human, for example, a patient having cancer and/or solid tumors.

As used herein, the terms “treatment”, “treat”, “treated”, or “treating” refer to prophylaxis and/or therapy. When used with respect to a solid tumor, such as a cancerous solid tumor, for example, the term refers to prevention by prophylactic treatment, which increases the subject's resistance to solid tumors or cancer. In some examples, the subject may be treated to prevent cancer, where the cancer is familial, or is genetically associated. When used with respect to an infectious disease, for example, the term refers to a prophylactic treatment which increases the resistance of a subject to infection with a pathogen or, in other words, decreases the likelihood that the subject will become infected with the pathogen or will show signs of illness attributable to the infection, as well as a treatment after the subject has become infected in order to fight the infection, for example, reduce or eliminate the infection or prevent it from becoming worse.

A typical dose of T cells for a subject is between 10⁴-10⁷ cells/kg. In some embodiments, a dose of T cells for a subject is between about 0.1×10⁶ cells/kg to about 9×10⁶ cells/kg. In some embodiments, a dose of T cells for a subject is between about 0.1×10⁶ cells/kg to about 8×10⁶ cells/kg. In some embodiments, a dose of T cells for a subject is between about 0.1×10⁶ cells/kg to about 7.5×10⁶ cells/kg. In some embodiments, a dose of T cells for a subject is between about 0.1×10⁶ cells/kg to about 7×10⁶ cells/kg. In some embodiments, a dose of T cells for a subject is between about 0.1×10⁶ cells/kg to about 6×10⁶ cells/kg. In some embodiments, a dose of T cells for a subject is between about 0.3×10⁶ cells/kg to about 5×10⁶ cells/kg. In some embodiments, a dose of T cells for a subject is between about 1.25×10⁶ cells/kg to about 2.5×10⁶ cells/kg. In some embodiments, a dose of T cells for a subject is about 0.3×10⁶ cells/kg (±20%). In some embodiments, a dose of T cells for a subject is about 0.625×10⁶ cells/kg (±20%). In some embodiments, a dose of T cells for a subject is about 1.25×10⁶ cells/kg (±20%). In some embodiments, a dose of T cells for a subject is about 2.5×10⁶ cells/kg (±20%). In some embodiments, a dose of T cells for a subject is about 5×10⁶ cells/kg (±20%). In some embodiments, a dose of T cells for a subject is about 6×10⁶ cells/kg (±20%). In some embodiments, a dose of T cells for a subject is about 7×10⁶ cells/kg (±20%). In some embodiments, a dose of T cells for a subject is about 7.5×10⁶ cells/kg (±20%).

The recipient may undergo myeloablative conditioning prior to receiving the modified cell population comprising genetically-modified T cells. Thus, the recipient's own α/β T cells (and B cells) can be depleted prior to receiving the genetically-modified T cells. Similarly, haematopoietic (stem) cells which are administered to a recipient may be depleted for α/β cells. In contrast, genetically-modified donor T cells administered to the recipient are generally not depleted for α/β cells.

In some embodiments a lymphodepleting chemotherapy regimen is administered before the infusion of the modified T cells. In some embodiments, lymphodepleting chemotherapy regimen comprises cyclophosphamide 500 mg/m² intravenously and fludarabine 30 mg/m² intravenously is administered on the fifth, fourth, and third day before infusion of PSCA CAR T cells, unless the combination is not tolerated. If it is determined that combination-based lymphodepletion presents an unfavorable safety risk to a subject, then lymphodepletion may proceed with cyclophosphamide alone.

The recipient can be a child e.g. a child aged from 0-16 years old, or from 0-10 years old. In some embodiments, the recipient is an adult.

Subjects receiving the genetically-modified T cells may also receive other tissue from an allogeneic donor e.g. they can receive haematopoietic cells and/or haematopoietic stem cells (e.g. CD34+ cells). This allograft tissue and the genetically-modified T cells are ideally derived from the same donor, such that they will be genetically matched. In some embodiments, the donor and the recipient are a matched unrelated donor, or a suitable family member. For instance, the donor may be the recipient's parent or child. Where a subject is identified as being in need of genetically-modified T cells, therefore, a suitable donor can be identified as a T cell donor.

Where modified cell populations provided herein, for example, modified cell populations comprising modified T cells, are used in conjunction with haematopoietic cells and/or haematopoietic stem cells, the modified cell populations may, in some examples, be administered at a later timepoint e.g. between 20-100 days later.

In certain embodiments, the present methods utilize the technique of chemically induced dimerization (CID) to produce a conditionally provided costimulation to the modified cells. In addition to this technique being inducible, it also is reversible, due to the degradation of the labile dimerizing agent.

In some embodiments, the ligand is a small molecule. Often, the ligand is dimeric, sometimes, the ligand is a dimeric FK506 or a dimeric FK506 analog. In certain embodiments, the ligand is AP1903 (CAS Index Name: 2-Piperidinecarboxylic acid, 1-[(2S)-1-oxo-2-(3,4,5-trimethoxyphenyl)butyl]-, 1,2-ethanediylbis [imino(2-oxo-2,1-ethanediyl)oxy-3,1-phenylene[(1R)-3-(3,4-Dimethoxyphenyl)propylidene]]ester, [2S-[1(R*),2R*[S*[S*[1(R*),2R*]]]]]-(9Cl) CAS Registry Number: 195514-63-7; Molecular Formula: C78H98N4O20 Molecular Weight: 1411.65). In certain embodiments, the ligand is AP20187. In certain embodiments, the ligand is an AP20187 analog, such as, for example, AP1510. In some embodiments, certain analogs will be appropriate for the FKBP12, and certain analogs appropriate for the wobbled version of FKBP12. In certain embodiments, one ligand binding region is included in the chimeric protein. In other embodiments, two or more ligand binding regions are included. Where, for example, the ligand binding region is FKBP12, where two of these regions are included, one may, for example, be the wobbled version.

In some embodiments the dose of rimiducid is between 0.01 mg/kg-1.0 mg/kg. In some embodiments, a fixed dose of AP1903 for injection is used, for example, may be 0.4 mg/kg intravenously infused over 2 hours. The amount of AP1903 needed in vitro for effective signaling of cells is about 10-100 nM (MW: 1412 Da). This equates to 14-140 μg/L or ˜0.014-0.14 mg/kg (1.4-140 μg/kg). The dosage may vary according to the application, and may, in certain examples, be more in the range of 0.1-10 nM, or in the range of 50-150 nM, 10-200 nM, 75-125 nM, 100-500 nM, 100-600 nM, 100-700 nM, 100-800 nM, or 100-900 nM. The multimeric ligand or CID, such as, for example, AP1903 (rimiducid), may be delivered, for example at doses of about 0.01 to 1 mg/kg subject weight, of about 0.05 to 0.5 mg/kg subject weight, 0.1 to 2 mg/kg subject weight, of about 0.05 to 1.0 mg/kg subject weight, of about 0.1 to 5 mg/kg subject weight, of about 0.2 to 4 mg/kg subject weight, of about 0.3 to 3 mg/kg subject weight, of about 0.3 to 2 mg/kg subject weight, or about 0.3 to 1 mg/kg subject weight, for example, about 0.1, 0.2, 0.3, 0.4, 0.5, 0.6, 0.7, 0.8, 0.9, 1.0, 1.5, 2, 2.5, 3, 3.5, 4, 4.5, 5, 6, 7, 8, 9, or 10 mg/kg subject weight. In some embodiments, the ligand is provided at 0.4 mg/kg per dose, for example at a concentration of 5 mg/mL. Vials or other containers may be provided containing the ligand at, for example, a volume per vial of about 0.25 ml to about 10 ml, for example, about 0.25, 0.5, 1, 1.5, 2, 2.5, 3, 3.5, 4, 4.5, 5, 5.5, 6, 6.5, 7, 7.5, 8, 8.5, 9, 9.5, or 10 ml, for example, about 2 ml.

The administration schedule of the ligand (e.g. rimiducid) may be determined as appropriate for the patient and may, for example, comprise a dosing schedule where the modified T cells are administered at week 0, followed by induction by administration of the chemical inducer of dimerization, followed by administration of additional inducer when needed to obtain an effective therapeutic result or, for example, at 2, 4, 6, 8, 10, 12, 14, 16, 18, 20 intervals thereafter for a total of, for example, 2, 4, 6, 8, 10, 12, 14, 16, 18, 20, 22, 24, 26, 28, or 30, 40, 50, 60, 70, 80, 90, or 100 weeks.

The administration schedule of the ligand (e.g. rimiducid) may be determined as appropriate for the patient and may, for example, comprise a dosing schedule where the inducible MyD88/CD40 PSCA CAR-T cell is administered at week 0, followed by induction by administration of the chemical inducer of dimerization, followed by administration of additional inducer when needed to obtain an effective therapeutic result or, for example, at 2, 4, 6, 8, 10, 12, 14, 16, 18, 20 intervals thereafter for a total of, for example, 2, 4, 6, 8, 10, 12, 14, 16, 18, 20, 22, 24, 26, 28, or 30, 40, 50, 60, 70, 80, 90, or 100 weeks.

In some embodiments, for administration of transduced T cells, one dose of cells may be sufficient, followed by multiple doses of ligand. In some embodiments, T cells may be provided more than once. Therefore, for example, the administration schedule may be determined as appropriate for the patient and may, for example, comprise a dosing schedule where the T cells are administered at week 0, followed by induction by administration of the chemical inducer of dimerization, followed by administration of additional T cells and inducer at 2 week intervals thereafter for a total of, for example, 2, 4, 6, 8, 10, 12, 14, 16, 18, 20, 22, 24, 26, 28, or 30 weeks.

Other dosing schedules include, for example, a schedule where one dose of the cells and one dose of the inducer are administered. In another example, the schedule may comprise administering the cells and the inducer are administered at week 0, followed by the administration of additional cells and inducer at 4 week intervals, for a total of, for example, 4, 8, 12, 16, 20, 24, 28, or 32 weeks.

In another example, the schedule may comprise administering the cells one time (at week 0) and then administering the inducer (e.g., rimiducid) at week 0 together or after administrations of the cells. For example, the administration of the cells and inducer can be concurrent, or the inducer can be administered subsequently after the cells on the same day or the following day, or 2, 3, 4, 5, or days. In some embodiments, the first administration of the inducer is followed by the administration of additional inducer (e.g., rimiducid) at certain intervals (e.g., 1, 2, 3, or 4 week intervals). In some embodiments, the inducer (e.g., rimiducid) is administered to the subject once per day. In some embodiments, the inducer (e.g., rimiducid) is administered to the subject three times per week. In some embodiments, the inducer (e.g., rimiducid) is administered to the subject twice per week. In some embodiments, the inducer (e.g., rimiducid) is administered to the subject once per week. In some embodiments, the inducer (e.g., rimiducid) is administered to the subject once every other week. In some embodiments, the inducer (e.g., rimiducid) is administered to the subject twice per month. In some embodiments, the inducer (e.g., rimiducid) is administered to the subject once per month.

In another example, the schedule may comprise administering the cells one time (at week 0) and then administering the inducer (e.g., rimiducid) at week 1, followed by the administration of additional inducer (e.g., rimiducid) at certain intervals (e.g., 1, 2, 3, or 4 week intervals). In some embodiments, the inducer (e.g., rimiducid) is administered to the subject once per day. In some embodiments, the inducer (e.g., rimiducid) is administered to the subject three times per week. In some embodiments, the inducer (e.g., rimiducid) is administered to the subject twice per week. In some embodiments, the inducer (e.g., rimiducid) is administered to the subject once per week. In some embodiments, the inducer (e.g., rimiducid) is administered to the subject once every other week. In some embodiments, the inducer (e.g., rimiducid) is administered to the subject twice per month. In some embodiments, the inducer (e.g., rimiducid) is administered to the subject once per month.

In some embodiments, the administration schedule of the ligand (e.g. rimiducid) comprise a dosing schedule where the inducible MyD88/CD40 PSCA CAR-T cell is administered at week 0, followed by induction by administration of the chemical inducer of dimerization once per day.

In some embodiments, the administration schedule of the ligand (e.g. rimiducid) comprise a dosing schedule where the inducible MyD88/CD40 PSCA CAR-T cell is administered at week 0, followed by induction by administration of the chemical inducer of dimerization six times per week.

In some embodiments, the administration schedule of the ligand (e.g. rimiducid) comprise a dosing schedule where the inducible MyD88/CD40 PSCA CAR-T cell is administered at week 0, followed by induction by administration of the chemical inducer of dimerization five times per week.

In some embodiments, the administration schedule of the ligand (e.g. rimiducid) comprise a dosing schedule where the inducible MyD88/CD40 PSCA CAR-T cell is administered at week 0, followed by induction by administration of the chemical inducer of dimerization fourth times per week.

In some embodiments, the administration schedule of the ligand (e.g. rimiducid) comprise a dosing schedule where the inducible MyD88/CD40 PSCA CAR-T cell is administered at week 0, followed by induction by administration of the chemical inducer of dimerization three times per week.

In some embodiments, the administration schedule of the ligand (e.g. rimiducid) comprise a dosing schedule where the inducible MyD88/CD40 PSCA CAR-T cell is administered at week 0, followed by induction by administration of the chemical inducer of dimerization two times per week.

In some embodiments, the administration schedule of the ligand (e.g. rimiducid) comprise a dosing schedule where the inducible MyD88/CD40 PSCA CAR-T cell is administered at week 0, followed by induction by administration of the chemical inducer of dimerization once per week.

In some embodiments, the administration schedule of the ligand (e.g. rimiducid) comprise a dosing schedule where the inducible MyD88/CD40 PSCA CAR-T cell is administered at week 0, followed by induction by administration of the chemical inducer of dimerization twice per month.

In some embodiments, the administration schedule of the ligand (e.g. rimiducid) comprise a dosing schedule where the inducible MyD88/CD40 PSCA CAR-T cell is administered at week 0, followed by induction by administration of the chemical inducer of dimerization once per month.

In some embodiments, a subject is administered a dose of MyD88/CD40 PSCA CAR-T cells between about 0.1×10⁶ cells/kg to about 9×10⁶ cells/kg, followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week. In some embodiments, the PSCA CAR-T contains scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, a subject is administered a dose of MyD88/CD40 PSCA CAR-T cells between about 0.1×10⁶ cells/kg to about 8×10⁶ cells/kg, followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week. In some embodiments, the PSCA CAR-T contains scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, a subject is administered a dose of MyD88/CD40 PSCA CAR-T cells between about 0.1×10⁶ cells/kg to about 7.5×10⁶ cells/kg, followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week. In some embodiments, the PSCA CAR-T contains scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, a subject is administered a dose of MyD88/CD40 PSCA CAR-T cells between about 0.1×10⁶ cells/kg to about 7×10⁶ cells/kg, followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week. In some embodiments, the PSCA CAR-T contains scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, a subject is administered a dose of MyD88/CD40 PSCA CAR-T cells between about 0.1×10⁶ cells/kg to about 6×10⁶ cells/kg, followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week. In some embodiments, the PSCA CAR-T contains scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, a subject is administered a dose of MyD88/CD40 PSCA CAR-T cells between about 0.3×10⁶ cells/kg to about 5×10⁶ cells/kg, followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week. In some embodiments, the PSCA CAR-T contains scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, a subject is administered a dose of MyD88/CD40 PSCA CAR-T cells of about 1.25×10⁶ cells/kg to about 2.5×10⁶ cells/kg, followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week. In some embodiments, the PSCA CAR-T contains scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, a subject is administered a dose of MyD88/CD40 PSCA CAR-T cells of about 0.3×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week. In some embodiments, the PSCA CAR-T contains scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, a subject is administered a dose of MyD88/CD40 PSCA CAR-T cells of about 0.625×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week. In some embodiments, the PSCA CAR-T contains scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, a subject is administered a dose of MyD88/CD40 PSCA CAR-T cells of about 1.25×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month as long as necessary (e.g. cancer remission). In some embodiments, the dose of rimiducid is 0.4 mg/kg once a week. In some embodiments, the PSCA CAR-T contains scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, a subject is administered a dose of MyD88/CD40 PSCA CAR-T cells of about 2.5×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month as long as necessary (e.g. cancer remission). In some embodiments, the dose of rimiducid is 0.4 mg/kg once a week. In some embodiments, the PSCA CAR-T contains scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, a subject is administered a dose of MyD88/CD40 PSCA CAR-T cells of about 5×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week. In some embodiments, the PSCA CAR-T contains scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, a subject is administered a dose of MyD88/CD40 PSCA CAR-T cells of about 6×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week. In some embodiments, the PSCA CAR-T contains scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, a subject is administered a dose of MyD88/CD40 PSCA CAR-T cells of about 7×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week. In some embodiments, the PSCA CAR-T contains scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, a subject is administered a dose of MyD88/CD40 PSCA CAR-T cells of about 7.5×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week. In some embodiments, the PSCA

CAR-T contains scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, a subject is administered a dose of MyD88/CD40 PSCA CAR-T cells of about 8×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week. In some embodiments, the PSCA CAR-T contains scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, a subject is administered a dose of MyD88/CD40 PSCA CAR-T cells containing a scFV derived from A11 VL (SEQ ID NO: 49) and A11 VH (SEQ ID NO: 51) of about 0.3×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from A11 VL (SEQ ID NO: 49) and A11 VH (SEQ ID NO: 51) of about 0.625×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from A11 VL (SEQ ID NO: 49) and A11 VH (SEQ ID NO: 51) of about 1.25×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from A11 VL (SEQ ID NO: 49) and A11 VH (SEQ ID NO: 51) of about 2.5×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, or once a month as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from A11 VL (SEQ ID NO: 49) and A11 VH (SEQ ID NO: 51) of about 5×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from A11 VL (SEQ ID NO: 49) and A11 VH (SEQ ID NO: 51) of about 6×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from A11 VL (SEQ ID NO: 49) and A11 VH (SEQ ID NO: 51) of about 7×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from A11 VL (SEQ ID NO: 49) and A11 VH (SEQ ID NO: 51) of about 7.5×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from A11 VL (SEQ ID NO: 49) and A11 VH (SEQ ID NO: 51) of about 8×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from bm2B3 VL (SEQ ID NO:53) and bm2B3 VH (SEQ ID NO:55) of about 0.3×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from bm2B3 VL (SEQ ID NO:53) and bm2B3 VH (SEQ ID NO:55) of about 0.625×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from bm2B3 VL (SEQ ID NO:53) and bm2B3 VH (SEQ ID NO:55) of about 1.25×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from bm2B3 VL (SEQ ID NO:53) and bm2B3 VH (SEQ ID NO:55) of about 2.5×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from bm2B3 VL (SEQ ID NO:53) and bm2B3 VH (SEQ ID NO:55) of about 5×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg.

In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from bm2B3 VL (SEQ ID NO:53) and bm2B3 VH (SEQ ID NO:55) of about 6×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from bm2B3 VL (SEQ ID NO:53) and bm2B3 VH (SEQ ID NO:55) of about 7×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from bm2B3 VL (SEQ ID NO:53) and bm2B3 VH (SEQ ID NO:55) of about 7.5×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg.

In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from bm2B3 VL (SEQ ID NO:53) and bm2B3 VH (SEQ ID NO:55) of about 8×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from HA1-4.121 VH (SEQ ID NO: 57) and HLA1-4.121 VL (SEQ ID NO: 59) of about 0.3×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from HA1-4.121 VH (SEQ ID NO: 57) and HLA1-4.121 VL (SEQ ID NO: 59) of about 0.625×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from HA1-4.121 VH (SEQ ID NO: 57) and HLA1-4.121 VL (SEQ ID NO: 59) of about 1.25×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from HA1-4.121 VH (SEQ ID NO: 57) and HLA1-4.121 VL (SEQ ID NO: 59) of about 2.5×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from HA1-4.121 VH (SEQ ID NO: 57) and HLA1-4.121 VL (SEQ ID NO: 59) of about 5×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from HA1-4.121 VH (SEQ ID NO: 57) and HLA1-4.121 VL (SEQ ID NO: 59) of about 6×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from HA1-4.121 VH (SEQ ID NO: 57) and HLA1-4.121 VL (SEQ ID NO: 59) of about 7×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from HA1-4.121 VH (SEQ ID NO: 57) and HLA1-4.121 VL (SEQ ID NO: 59) of about 7.5×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from HA1-4.121 VH (SEQ ID NO: 57) and HLA1-4.121 VL (SEQ ID NO: 59) of about 8×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from HA1-4.121 VH H1-1.10 VH (SEQ ID NO: 61) and H1-1.10 VL (SEQ ID NO: 63) of about 0.3×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from H1-1.10 VH (SEQ ID NO: 61) and H1-1.10 VL (SEQ ID NO: 63) of about 0.625×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from H1-1.10 VH (SEQ ID NO: 61) and H1-1.10 VL (SEQ ID NO: 63) of about 1.25×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from H1-1.10 VH (SEQ ID NO: 61) and H1-1.10 VL (SEQ ID NO: 63) of about 2.5×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from H1-1.10 VH (SEQ ID NO: 61) and H1-1.10 VL (SEQ ID NO: 63) of about 5×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg.

In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from H1-1.10 VH (SEQ ID NO: 61) and H1-1.10 VL (SEQ ID NO: 63) of about 6×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from H1-1.10 VH (SEQ ID NO: 61) and H1-1.10 VL (SEQ ID NO: 63) of about 7×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from H1-1.10 VH (SEQ ID NO: 61) and H1-1.10 VL (SEQ ID NO: 63) of about 7.5×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from H1-1.10 VH (SEQ ID NO: 61) and H1-1.10 VL (SEQ ID NO: 63) of about 8×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from 1G8 (2B3) (SEQ ID NO: 65) and 1G8 (2B3-05) (SEQ ID NO: 67) of about 0.3×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from 1G8 (2B3) (SEQ ID NO: 65) and 1G8 (2B3-05) (SEQ ID NO: 67) of about 0.625×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from 1G8 (2B3) (SEQ ID NO: 65) and 1G8 (2B3-05) (SEQ ID NO: 67) of about 1.25×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from 1G8 (2B3) (SEQ ID NO: 65) and 1G8 (2B3-05) (SEQ ID NO: 67) of about 2.5×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from 1G8 (2B3) (SEQ ID NO: 65) and 1G8 (2B3-05) (SEQ ID NO: 67) of about 5×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from 1G8 (2B3) (SEQ ID NO: 65) and 1G8 (2B3-05) (SEQ ID NO: 67) of about 6×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from 1G8 (2B3) (SEQ ID NO: 65) and 1G8 (2B3-05) (SEQ ID NO: 67) of about 7×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from 1G8 (2B3) (SEQ ID NO: 65) and 1G8 (2B3-05) (SEQ ID NO: 67) of about 7.5×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from 1G8 (2B3) (SEQ ID NO: 65) and 1G8 (2B3-05) (SEQ ID NO: 67) of about 8×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from 1G8 (2B3-A2) (SEQ ID NO: 69) and 1G8 (2B3-A11) (SEQ ID NO: 71) of about 0.3×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from 1G8 (2B3-A2) (SEQ ID NO: 69) and 1G8 (2B3-A11) (SEQ ID NO: 71) of about 0.625×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from 1G8 (2B3-A2) (SEQ ID NO: 69) and 1G8 (2B3-A11) (SEQ ID NO: 71) of about 1.25×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from 1G8 (2B3-A2) (SEQ ID NO: 69) and 1G8 (2B3-A11) (SEQ ID NO: 71) of about 2.5×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from 1G8 (2B3-A2) (SEQ ID NO: 69) and 1G8 (2B3-A11) (SEQ ID NO: 71) of about 5×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from 1G8 (2B3-A2) (SEQ ID NO: 69) and 1G8 (2B3-A11) (SEQ ID NO: 71) of about 6×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from 1G8 (2B3-A2) (SEQ ID NO: 69) and 1G8 (2B3-A11) (SEQ ID NO: 71) of about 7×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from 1G8 (2B3-A2) (SEQ ID NO: 69) and 1G8 (2B3-A11) (SEQ ID NO: 71) of about 7.5×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from 1G8 (2B3-A2) (SEQ ID NO: 69) and 1G8 (2B3-A11) (SEQ ID NO: 71) of about 8×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, the modified T cells are administered with unmodified polyclonal T cells. The unmodified polyclonal T cells can be administered in an amount of about 0.1×10⁶ cells/kg to about 30×10⁶ cells/kg. The polyclonal T cells can be administered at 0.1×10⁶, 0.5×10⁶, 1×10⁶, 1.25×10⁶, 2×10⁶, 3×10⁶, 4×10⁶, 5×10⁶, 10×10⁶, 15×10⁶, 20×10⁶, 25×10⁶ or 30×10⁶ cells/kg.

In some embodiments, a subject is administered a dose of modified T cells between about 0.1×10⁶ cells/kg to about 9×10⁶ cells/kg in combination with unmodified polyclonal T cells in an amount between 0.1×10⁶ cells/kg to about 30×10⁶ cells/kg. The T cells administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week. In some embodiments, the modified T cells are MyD88/CD40 PSCA CAR-T cells containing a scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, a subject is administered a dose of modified T cells between about 0.1×10⁶ cells/kg to about 8×10⁶ cells/kg in combination with unmodified polyclonal T cells in an amount between 0.1×10⁶ cells/kg to about 30×10⁶ cells/kg. The T cells administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week. In some embodiments, the modified T cells are MyD88/CD40 PSCA CAR-T cells containing a scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, a subject is administered a dose of modified T cells between about 0.1×10⁶ cells/kg to about 7.5×10⁶ cells/kg in combination with unmodified polyclonal T cells in an amount between 0.1×10⁶ cells/kg to about 30×10⁶ cells/kg. The T cells administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week. In some embodiments, the modified T cells are MyD88/CD40 PSCA CAR-T cells containing a scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, a subject is administered a dose of modified T cells between about 0.1×10⁶ cells/kg to about 7×10⁶ cells/kg in combination with unmodified polyclonal T cells in an amount between 0.1×10⁶ cells/kg to about 30×10⁶ cells/kg. The T cells administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week. In some embodiments, the modified T cells are MyD88/CD40 PSCA CAR-T cells containing a scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, a subject is administered a dose of modified T cells between about 0.1×10⁶ cells/kg to about 6×10⁶ cells/kg in combination with unmodified polyclonal T cells in an amount between 0.1×10⁶ cells/kg to about 30×10⁶ cells/kg. The T cells administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week. In some embodiments, the modified T cells are MyD88/CD40 PSCA CAR-T cells containing a scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, a subject is administered a dose of modified T cells between about 0.3×10⁶ cells/kg to about 5×10⁶ cells/kg in combination with unmodified polyclonal T cells in an amount between 0.1×10⁶ cells/kg to 25×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week. In some embodiments, the modified T cells are MyD88/CD40 PSCA CAR-T cells containing a scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, a subject is administered a dose of modified T cells of about 1.25×10⁶ cells/kg to about 2.5×10⁶ cells/kg in combination with unmodified polyclonal T cells in an amount between 0.1×10⁶ cells/kg to 20×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week. In some embodiments, the modified T cells are MyD88/CD40 PSCA CAR-T cells containing a scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, a subject is administered a dose of modified T cells of about 1.25×10⁶ cells/kg (±20%) in combination with unmodified polyclonal T cells in an amount between 0.1×10⁶ cells/kg to 20×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week. In some embodiments, the modified T cells are MyD88/CD40 PSCA CAR-T cells containing a scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, a subject is administered a dose of modified T cells of about 2.5×10⁶ cells/kg (±20%) in combination with unmodified polyclonal T cells in an amount between 0.1×10⁶ cells/kg to 25×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week. In some embodiments, the modified T cells are MyD88/CD40 PSCA CAR-T cells containing a scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, a subject is administered a dose of modified T cells of about 5×10⁶ cells/kg (±20%) in combination with unmodified polyclonal T cells in an amount between 0.1×10⁶ cells/kg to 30×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week. In some embodiments, the modified T cells are MyD88/CD40 PSCA CAR-T cells containing a scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, a subject is administered a dose of modified T cells of about 6×10⁶ cells/kg (±20%) in combination with unmodified polyclonal T cells in an amount between 0.1×10⁶ cells/kg to 30×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week. In some embodiments, the modified T cells are MyD88/CD40 PSCA CAR-T cells containing a scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, a subject is administered a dose of modified T cells of about 7×10⁶ cells/kg (±20%) in combination with unmodified polyclonal T cells in an amount between 0.1×10⁶ cells/kg to 30×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week. In some embodiments, the modified T cells are MyD88/CD40 PSCA CAR-T cells containing a scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, a subject is administered a dose of modified T cells of about 7.5×10⁶ cells/kg (±20%) in combination with unmodified polyclonal T cells in an amount between 0.1×10⁶ cells/kg to 30×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week. In some embodiments, the modified T cells are MyD88/CD40 PSCA CAR-T cells containing a scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, a subject is administered a dose of modified T cells of about 8×10⁶ cells/kg (±20%) in combination with unmodified polyclonal T cells in an amount between 0.1×10⁶ cells/kg to 30×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week. In some embodiments, the modified T cells are MyD88/CD40 PSCA CAR-T cells containing a scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, a subject is administered a dose of MyD88/CD40 PSCA CAR-T cells containing a scFV derived from A11 VL (SEQ ID NO: 49) and A11 VH (SEQ ID NO: 51) of about 1.25×10⁶ cells/kg (±20%) in combination with unmodified polyclonal T cells in an amount between 0.1×10⁶ cells/kg to 20×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from A11 VL (SEQ ID NO: 49) and A11 VH (SEQ ID NO: 51) of about 2.5×10⁶ cells/kg (±20%) in combination with unmodified polyclonal T cells in an amount between 0.1×10⁶ cells/kg to 25×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from A11 VL (SEQ ID NO: 49) and A11 VH (SEQ ID NO: 51) of about 5×10⁶ cells/kg (±20%) in combination with unmodified polyclonal T cells in an amount between 0.1×10⁶ cells/kg to 30×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from A11 VL (SEQ ID NO: 49) and A11 VH (SEQ ID NO: 51) of about 6×10⁶ cells/kg (±20%) in combination with unmodified polyclonal T cells in an amount between 0.1×10⁶ cells/kg to 30×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from A11 VL (SEQ ID NO: 49) and A11 VH (SEQ ID NO: 51) of about 7×10⁶ cells/kg (±20%) in combination with unmodified polyclonal T cells in an amount between 0.1×10⁶ cells/kg to 30×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from A11 VL (SEQ ID NO: 49) and A11 VH (SEQ ID NO: 51) of about 7.5×10⁶ cells/kg (±20%) in combination with unmodified polyclonal T cells in an amount between 0.1×10⁶ cells/kg to 30×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from A11 VL (SEQ ID NO: 49) and A11 VH (SEQ ID NO: 51) of about 8×10⁶ cells/kg (±20%) in combination with unmodified polyclonal T cells in an amount between 0.1×10⁶ cells/kg to 30×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from bm2B3 VL (SEQ ID NO:53) and bm2B3 VH (SEQ ID NO:55) of about 1.25×10⁶ cells/kg (±20%) in combination with unmodified polyclonal T cells in an amount between 0.1×10⁶ cells/kg to 20×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from bm2B3 VL (SEQ ID NO:53) and bm2B3 VH (SEQ ID NO:55) of about 2.5×10⁶ cells/kg (±20%) in combination with unmodified polyclonal T cells in an amount between 0.1×10⁶ cells/kg to 25×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from bm2B3 VL (SEQ ID NO:53) and bm2B3 VH (SEQ ID NO:55) of about 5×10⁶ cells/kg (±20%) in combination with unmodified polyclonal T cells in an amount between 0.1×10⁶ cells/kg to 30×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from bm2B3 VL (SEQ ID NO:53) and bm2B3 VH (SEQ ID NO:55) of about 6×10⁶ cells/kg (±20%) in combination with unmodified polyclonal T cells in an amount between 0.1×10⁶ cells/kg to 30×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from bm2B3 VL (SEQ ID NO:53) and bm2B3 VH (SEQ ID NO:55) of about 7×10⁶ cells/kg (±20%) in combination with unmodified polyclonal T cells in an amount between 0.1×10⁶ cells/kg to 30×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from bm2B3 VL (SEQ ID NO:53) and bm2B3 VH (SEQ ID NO:55) of about 7.5×10⁶ cells/kg (±20%) in combination with unmodified polyclonal T cells in an amount between 0.1×10⁶ cells/kg to 30×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from bm2B3 VL (SEQ ID NO:53) and bm2B3 VH (SEQ ID NO:55) of about 8×10⁶ cells/kg (±20%) in combination with unmodified polyclonal T cells in an amount between 0.1×10⁶ cells/kg to 30×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from HA1-4.121 VH (SEQ ID NO: 57) and HLA1-4.121 VL (SEQ ID NO: 59) of about 1.25×10⁶ cells/kg (±20%) in combination with unmodified polyclonal T cells in an amount between 0.1×10⁶ cells/kg to 20×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from HA1-4.121 VH (SEQ ID NO: 57) and HLA1-4.121 VL (SEQ ID NO: 59) of about 2.5×10⁶ cells/kg (±20%) in combination with unmodified polyclonal T cells in an amount between 0.1×10⁶ cells/kg to 25×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from HA1-4.121 VH (SEQ ID NO: 57) and HLA1-4.121 VL (SEQ ID NO: 59) of about 5×10⁶ cells/kg (±20%) in combination with unmodified polyclonal T cells in an amount between 0.1×10⁶ cells/kg to 30×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from HA1-4.121 VH (SEQ ID NO: 57) and HLA1-4.121 VL (SEQ ID NO: 59) of about 6×10⁶ cells/kg (±20%) in combination with unmodified polyclonal T cells in an amount between 0.1×10⁶ cells/kg to 30×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from HA1-4.121 VH (SEQ ID NO: 57) and HLA1-4.121 VL (SEQ ID NO: 59) of about 7×10⁶ cells/kg (±20%) in combination with unmodified polyclonal T cells in an amount between 0.1×10⁶ cells/kg to 30×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from HA1-4.121 VH (SEQ ID NO: 57) and HLA1-4.121 VL (SEQ ID NO: 59) of about 7.5×10⁶ cells/kg (±20%) in combination with unmodified polyclonal T cells in an amount between 0.1×10⁶ cells/kg to 30×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from HA1-4.121 VH (SEQ ID NO: 57) and HLA1-4.121 VL (SEQ ID NO: 59) of about 8×10⁶ cells/kg (±20%) in combination with unmodified polyclonal T cells in an amount between 0.1×10⁶ cells/kg to 30×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from H1-1.10 VH (SEQ ID NO: 61) and H1-1.10 VL (SEQ ID NO: 63) of about 1.25×10⁶ cells/kg (±20%) in combination with unmodified polyclonal T cells in an amount between 0.1×10⁶ cells/kg to 20×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from H1-1.10 VH (SEQ ID NO: 61) and H1-1.10 VL (SEQ ID NO: 63) of about 2.5×10⁶ cells/kg (±20%) in combination with unmodified polyclonal T cells in an amount between 0.1×10⁶ cells/kg to 25×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from H1-1.10 VH (SEQ ID NO: 61) and H1-1.10 VL (SEQ ID NO: 63) of about 5×10⁶ cells/kg (±20%) in combination with unmodified polyclonal T cells in an amount between 0.1×10⁶ cells/kg to 30×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from H1-1.10 VH (SEQ ID NO: 61) and H1-1.10 VL (SEQ ID NO: 63) of about 6×10⁶ cells/kg (±20%) in combination with unmodified polyclonal T cells in an amount between 0.1×10⁶ cells/kg to 30×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from H1-1.10 VH (SEQ ID NO: 61) and H1-1.10 VL (SEQ ID NO: 63) of about 7×10⁶ cells/kg (±20%) in combination with unmodified polyclonal T cells in an amount between 0.1×10⁶ cells/kg to 30×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from H1-1.10 VH (SEQ ID NO: 61) and H1-1.10 VL (SEQ ID NO: 63) of about 7.5×10⁶ cells/kg (±20%) in combination with unmodified polyclonal T cells in an amount between 0.1×10⁶ cells/kg to 30×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from H1-1.10 VH (SEQ ID NO: 61) and H1-1.10 VL (SEQ ID NO: 63) of about 8×10⁶ cells/kg (±20%) in combination with unmodified polyclonal T cells in an amount between 0.1×10⁶ cells/kg to 30×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from 1G8 (2B3) (SEQ ID NO: 65) and 1G8 (2B3-05) (SEQ ID NO: 67) of about 1.25×10⁶ cells/kg (±20%) in combination with unmodified polyclonal T cells in an amount between 0.1×10⁶ cells/kg to 20×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from 1G8 (2B3) (SEQ ID NO: 65) and 1G8 (2B3-05) (SEQ ID NO: 67) of about 2.5×10⁶ cells/kg (±20%) in combination with unmodified polyclonal T cells in an amount between 0.1×10⁶ cells/kg to 25×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from 1G8 (2B3) (SEQ ID NO: 65) and 1G8 (2B3-05) (SEQ ID NO: 67) of about 5×10⁶ cells/kg (±20%) in combination with unmodified polyclonal T cells in an amount between 0.1×10⁶ cells/kg to 30×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from 1G8 (2B3) (SEQ ID NO: 65) and 1G8 (2B3-05) (SEQ ID NO: 67) of about 6×10⁶ cells/kg (±20%) in combination with unmodified polyclonal T cells in an amount between 0.1×10⁶ cells/kg to 30×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from 1G8 (2B3) (SEQ ID NO: 65) and 1G8 (2B3-05) (SEQ ID NO: 67) of about 7×10⁶ cells/kg (±20%) in combination with unmodified polyclonal T cells in an amount between 0.1×10⁶ cells/kg to 30×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from 1G8 (2B3) (SEQ ID NO: 65) and 1G8 (2B3-05) (SEQ ID NO: 67) of about 7.5×10⁶ cells/kg (±20%) in combination with unmodified polyclonal T cells in an amount between 0.1×10⁶ cells/kg to 30×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from 1G8 (2B3) (SEQ ID NO: 65) and 1G8 (2B3-05) (SEQ ID NO: 67) of about 8×10⁶ cells/kg (±20%) in combination with unmodified polyclonal T cells in an amount between 0.1×10⁶ cells/kg to 30×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from 1G8 (2B3-A2) (SEQ ID NO: 69) and 1G8 (2B3-A11) (SEQ ID NO: 71) of about 1.25×10⁶ cells/kg (±20%) in combination with unmodified polyclonal T cells in an amount between 0.1×10⁶ cells/kg to 20×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from 1G8 (2B3-A2) (SEQ ID NO: 69) and 1G8 (2B3-A11) (SEQ ID NO: 71) of about 2.5×10⁶ cells/kg (±20%) in combination with unmodified polyclonal T cells in an amount between 0.1×10⁶ cells/kg to 25×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from 1G8 (2B3-A2) (SEQ ID NO: 69) and 1G8 (2B3-A11) (SEQ ID NO: 71) of about 5×10⁶ cells/kg (±20%) in combination with unmodified polyclonal T cells in an amount between 0.1×10⁶ cells/kg to 30×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from 1G8 (2B3-A2) (SEQ ID NO: 69) and 1G8 (2B3-A11) (SEQ ID NO: 71) of about 6×10⁶ cells/kg (±20%) in combination with unmodified polyclonal T cells in an amount between 0.1×10⁶ cells/kg to 30×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from 1G8 (2B3-A2) (SEQ ID NO: 69) and 1G8 (2B3-A11) (SEQ ID NO: 71) of about 7×10⁶ cells/kg (±20%) in combination with unmodified polyclonal T cells in an amount between 0.1×10⁶ cells/kg to 30×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from 1G8 (2B3-A2) (SEQ ID NO: 69) and 1G8 (2B3-A11) (SEQ ID NO: 71) of about 7.5×10⁶ cells/kg (±20%) in combination with unmodified polyclonal T cells in an amount between 0.1×10⁶ cells/kg to 30×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

In some embodiments, a subject is administered a dose of MyD88/CD40 PSCA CAR-T cells containing a scFV derived from 1G8 (2B3-A2) (SEQ ID NO: 69) and 1G8 (2B3-A11) (SEQ ID NO: 71) of about 8×10⁶ cells/kg (±20%) in combination with unmodified polyclonal T cells in an amount between 0.1×10⁶ cells/kg to 30×10⁶ cells/kg. The T cell administration is followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week.

The present methods also encompass methods of treatment or prevention of a disease caused by a hyperproliferative disease.

Cancers, including solid tumors, which may be treated using the pharmaceutical composition include, but are not limited to prostate cancer, pancreatic cancer, gastric cancer, ovarian cancer, urinary bladder cancer, lung cancer, esophageal cancer, endometrial cancer, cervical cancer or breast cancer.

In some embodiments, the invention provides for the treatment or prevention of prostate cancer, pancreatic cancer and gastric cancer.

In some embodiments, the invention provides for the treatment or prevention of prostate cancer by administering to a subject a dose of modified T cells between about 0.1×10⁶ cells/kg to about 8×10⁶ cells/kg, followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week. In some embodiments, the modified T cells are administered in combination with unmodified polyclonal T cells in an amount between 0.1×10⁶ cells/kg to 30×10⁶ cells/kg. In some embodiments, the modified T cells are MyD88/CD40 PSCA CAR-T cells containing a scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, the invention provides for the treatment or prevention of prostate cancer by administering to a subject a dose of modified T cells of about 5×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week. In some embodiments, the modified T cells are administered in combination with unmodified polyclonal T cells in an amount between 0.1×10⁶ cells/kg to 30×10⁶ cells/kg. In some embodiments, the modified T cells are MyD88/CD40 PSCA CAR-T cells containing a scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, the invention provides for the treatment or prevention of prostate cancer by administering to a subject a dose of modified T cells of about 7×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week. In some embodiments, the modified T cells are administered in combination with unmodified polyclonal T cells in an amount between 0.1×10⁶ cells/kg to 30×10⁶ cells/kg. In some embodiments, the modified T cells are MyD88/CD40 PSCA CAR-T cells containing a scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, the invention provides for the treatment or prevention of pancreatic cancer by administering to a subject a dose of modified T cells between about 0.1×10⁶ cells/kg to about 8×10⁶ cells/kg, followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week. In some embodiments, the modified T cells are administered in combination with unmodified polyclonal T cells in an amount between 0.1×10⁶ cells/kg to 30×10⁶ cells/kg. In some embodiments, the modified T cells are MyD88/CD40 PSCA CAR-T cells containing a scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, the invention provides for the treatment or prevention of pancreatic cancer by administering to a subject a dose of modified T cells of about 7×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week. In some embodiments, the modified T cells are administered in combination with unmodified polyclonal T cells in an amount between 0.1×10⁶ cells/kg to 30×10⁶ cells/kg. In some embodiments, the modified T cells are MyD88/CD40 PSCA CAR-T cells containing a scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, the invention provides for the treatment or prevention of pancreatic cancer by administering to a subject a dose of modified T cells of about 5×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week. In some embodiments, the modified T cells are administered in combination with unmodified polyclonal T cells in an amount between 0.1×10⁶ cells/kg to 30×10⁶ cells/kg. In some embodiments, the modified T cells are MyD88/CD40 PSCA CAR-T cells containing a scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, the invention provides for the treatment or prevention of gastric cancer by administering to a subject a dose of modified T cells between about 0.1×10⁶ cells/kg to about 8×10⁶ cells/kg, followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week. In some embodiments, the modified T cells are administered in combination with unmodified polyclonal T cells in an amount between 0.1×10⁶ cells/kg to 30×10⁶ cells/kg. In some embodiments, the modified T cells are MyD88/CD40 PSCA CAR-T cells containing a scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, the invention provides for the treatment or prevention of gastric cancer by administering to a subject a dose of modified T cells of about 7×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week. In some embodiments, the modified T cells are administered in combination with unmodified polyclonal T cells in an amount between 0.1×10⁶ cells/kg to 30×10⁶ cells/kg. In some embodiments, the modified T cells are MyD88/CD40 PSCA CAR-T cells containing a scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, the invention provides for the treatment or prevention of gastric cancer by administering to a subject a dose of modified T cells of about 5×10⁶ cells/kg (±20%), followed by administration of rimiducid at intervals of daily, three times a week, two times a week, weekly, twice or once a month for as long as necessary (e.g. cancer remission). In some embodiments, the dose of rimiducid is 0.01 to 1.0 mg/kg. In some embodiments, the dose of rimiducid is 0.01 mg/kg, 0.05 mg/kg, 0.1 mg/kg, 0.5 mg/kg, or 1.0 mg/kg. In some embodiments, the dose of rimiducid is 0.4 mg/kg daily, three times a week, two times a week, weekly, twice or once a month for as long as necessary (e.g. cancer remission). In some embodiments, the dose of rimiducid is 0.4 mg/kg once a week. In some embodiments, the modified T cells are administered in combination with unmodified polyclonal T cells in an amount between 0.1×10⁶ cells/kg to 30×10⁶ cells/kg. In some embodiments, the modified T cells are MyD88/CD40 PSCA CAR-T cells containing a scFV derived from the variable heavy chain and variable light chain from the PSCA antibodies selected from the group consisting of A11 VL (SEQ ID NO: 49), A11 VH (SEQ ID NO: 51); bm2B3 VL (SEQ ID NO:53), bm2B3 VH (SEQ ID NO:55); HA1-4.121 VH (SEQ ID NO: 57), HLA1-4.121 VL (SEQ ID NO: 59); H1-1.10 VH (SEQ ID NO: 61), H1-1.10 VL (SEQ ID NO: 63); 1G8 (2B3) (SEQ ID NO: 65), 1G8 (2B3-05) (SEQ ID NO: 67); 1G8 (2B3-A2) (SEQ ID NO: 69), and 1G8 (2B3-A11) (SEQ ID NO: 71).

In some embodiments, copy number of a tumor marker is monitored in a subject. In some embodiments, a reduction in the copy number of a tumor marker is measured. For example, this may include a reduction in the in copy number of a tumor marker of about 5%, 10%, 20%, 30%, 40%, 50%, 60%, 70%, 80%, 90%, or 100%. In some embodiments, copy number of prostate stem cell antigen (PSCA) is monitored in a subject. In some embodiments, a reduction in the copy number of prostate stem cell antigen (PSCA) is measured. For example, this may include a reduction in the in copy number of prostate stem cell antigen (PSCA) of about 5%, 10%, 20%, 30%, 40%, 50%, 60%, 70%, 80%, 90%, or 100%.

In some embodiments, tumor burden is monitored in a subject. Tumor burden may refer to the number of cancer cells, the size of a tumor, and/or the amount of cancer in the body. Tumor burden may be referred to as tumor load. In some embodiments, a reduction in tumor burden is measured. For example, this may include a reduction in tumor burden of about 5%, 10%, 20%, 30%, 40%, 50%, 60%, 70%, 80%, 90%, or 100%.

By “reducing tumor size” or “inhibiting tumor growth” or “reduction in tumor burden” of a solid tumor is meant a response to treatment, or stabilization of disease, according to standard guidelines, such as, for example, the Response Evaluation Criteria in Solid Tumors (RECIST) criteria. For example, this may include a reduction in the diameter of a solid tumor of about 5%, 10%, 20%, 30%, 40%, 50%, 60%, 70%, 80%, 90%, or 100%, or the reduction in the number of tumors, circulating tumor cells, or tumor markers, of about 5%, 10%, 20%, 30%, 40%, 50%, 60%, 70%, 80%, 90%, or 100%. The size of tumors may be analyzed by any method, including, for example, CT scan, MRI, for example, CT-MRI, chest X-ray (for tumors of the lung), or molecular imaging, for example, PET scan, such as, for example, a PET scan after administering an iodine 123-labelled PSA, for example, PSMA ligand, such as, for example, where the inhibitor is TROFEX™/MIP-1072/1095, or molecular imaging, for example, SPECT, or a PET scan using PSA, for example, PSMA antibody, such as, for example, capromad pendetide (Prostascint), a 111-iridium labeled PSMA antibody.

By “reducing, slowing, or inhibiting tumor vascularization” is meant a reduction in tumor vascularization of about 5%, 10%, 20%, 30%, 40%, 50%, 60%, 70%, 80%, 90%, or 100%, or a reduction in the appearance of new vasculature of about 5%, 10%, 20%, 30%, 40%, 50%, 60%, 70%, 80%, 90%, or 100%, when compared to the amount of tumor vascularization before treatment. The reduction may refer to one tumor, or may be a sum or an average of the vascularization in more than one tumor. Methods of measuring tumor vascularization include, for example, CAT scan, MRI, for example, CT-MRI, or molecular imaging, for example, SPECT, or a PET scan, such as, for example, a PET scan after administering an iodine 123-labelled PSA, for example, PSMA ligand, such as, for example, where the inhibitor is TROFEX™/MIP-1072/1095, or a PET scan using PSA, for example, PSMA antibody, such as, for example, capromad pendetide (Prostascint), a 111-iridium labeled PSMA antibody.

In some embodiments, progression of cancer (e.g., prostate cancer, pancreatic cancer, gastric cancer, ovarian cancer) is prevented or delayed at least 6 months. In some embodiments, progression of cancer is prevented or delayed at least 12 months. In some embodiments, the cancer (e.g., prostate cancer) has a Gleason score of 7, 8, 9, 10, or greater. In some embodiments, the subject has a partial or complete response by 3 months after administration of the inducible MyD88/CD40 PSCA CAR-T cells. In some embodiments, the subject has a partial or complete response by 3 months after administration of the CID (e.g., rimiducid). In some embodiments, the subject has a partial or complete response by 6 months after administration of the inducible MyD88/CD40 PSCA CAR-T cells. In some embodiments, the subject has a partial or complete response by 6 months after administration of the CID (e.g., rimiducid). In some embodiments, the subject has a partial or complete response by 9 months after administration of the inducible MyD88/CD40 PSCA CAR-T cells. In some embodiments, the subject has a partial or complete response by 9 months after administration of the CID (e.g., rimiducid).

A tumor is classified, or named as part of an organ (e.g., prostate gland, pancreas, stomach, ovary), when the tumor is present in the organ, or has derived from or metastasized from a tumor in the organ, or produces one or more organ-specific tumor markers. A tumor has metastasized from a tumor in the organ, when, for example, it is determined that the tumor has chromosomal breakpoints that are the same as, or similar to, a tumor in the organ of the subject. In certain instances a tumor (e.g., a prostate tumor; a pancreatic tumor, a gastric tumor, an ovarian tumor) expresses prostate stem cell antigen (PSCA).

A tumor is classified, or named as part of an organ, such as a prostate cancer tumor when, for example, the tumor is present in the prostate gland, or has derived from or metastasized from a tumor in the prostate gland, or produces PSA. A tumor has metastasized from a tumor in the prostate gland, when, for example, it is determined that the tumor has chromosomal breakpoints that are the same as, or similar to, a tumor in the prostate gland of the subject.

An indication of adjusting or maintaining a subsequent drug dose, such as, for example, a subsequent dose of the modified T cells, and/or a subsequent CID (e.g., rimiducid) dosage, can be provided in any convenient manner. An indication may be provided in tabular form (e.g., in a physical or electronic medium) in some embodiments. For example, the size of the tumor cell, or the number or level of tumor cells in a sample may be provided in a table, and a clinician may compare the symptoms with a list or table of stages of the disease. The clinician then can identify from the table an indication for subsequent drug dose. In certain embodiments, an indication can be presented (e.g., displayed) by a computer, after the symptoms are provided to the computer (e.g., entered into memory on the computer). For example, this information can be provided to a computer (e.g., entered into computer memory by a user or transmitted to a computer via a remote device in a computer network), and software in the computer can generate an indication for adjusting or maintaining a subsequent drug dose, and/or provide the subsequent drug dose amount.

Once a subsequent dose is determined based on the indication, a clinician may administer the subsequent dose or provide instructions to adjust the dose to another person or entity. The term “clinician” as used herein refers to a decision maker, and a clinician is a medical professional in certain embodiments. A decision maker can be a computer or a displayed computer program output in some embodiments, and a health service provider may act on the indication or subsequent drug dose displayed by the computer. A decision maker may administer the subsequent dose directly (e.g., infuse the subsequent dose into the subject) or remotely (e.g., pump parameters may be changed remotely by a decision maker).

Treatment for solid tumor cancers, including, for example, prostate cancer, pancreatic cancer, gastric cancer, ovarian cancer) may be optimized by determining the concentration of a biomarker associated with the tumor (e.g., PSCA), during the course of treatment. Because patients may have different responses to the course of treatment, the response to treatment may be monitored by following biomarker concentrations or levels in various body fluids or tissues. The determination of the concentration, level, or amount of a biomarker polypeptide may include detection of the full length polypeptide, or a fragment or variant thereof. The fragment or variant may be sufficient to be detected by, for example, immunological methods, mass spectrometry, nucleic acid hybridization, and the like. Optimizing treatment for individual patients may help to avoid side effects as a result of overdosing, may help to determine when the treatment is ineffective and to change the course of treatment, or may help to determine when doses may be increased, or to determine the timing of treatment.

For example, it has been determined that amount or concentration of certain biomarkers (e.g., PSCA) changes during the course of treatment of solid tumors. Predetermined target levels of such biomarkers, or biomarker thresholds may be identified in normal subject, are provided, which allow a clinician to determine whether a subsequent dose of a drug administered to a subject in need thereof, such as a subject with a solid tumor, such as, for example, a prostate tumor, pancreatic tumor, gastric tumor, ovarian tumor, may be increased, decreased or maintained. A clinician can make such a determination based on whether the presence, absence or amount of a biomarker is below, above or about the same as a biomarker threshold, respectively, in certain embodiments.

Where clinical applications are contemplated, it will be necessary to prepare pharmaceutical compositions—expression constructs, expression vectors, fused proteins, transduced cells, activated T cells, transduced and loaded T cells—in a form appropriate for the intended application.

Combination Therapies

In order to increase the effectiveness of the expression vectors presented herein, it may be desirable to combine these compositions and methods with an agent effective in the treatment of the disease.

In certain embodiments, anti-cancer agents may be used in combination with the present methods. An “anti-cancer” agent is capable of negatively affecting cancer in a subject, for example, by killing one or more cancer cells, inducing apoptosis in one or more cancer cells, reducing the growth rate of one or more cancer cells, reducing the incidence or number of metastases, reducing a tumor's size, inhibiting a tumor's growth, reducing the blood supply to a tumor or one or more cancer cells, promoting an immune response against one or more cancer cells or a tumor, preventing or inhibiting the progression of a cancer, or increasing the lifespan of a subject with a cancer. Anti-cancer agents include, for example, chemotherapy agents (chemotherapy), radiotherapy agents (radiotherapy), a surgical procedure (surgery), immune therapy agents (immunotherapy), genetic therapy agents (gene therapy), hormonal therapy, other biological agents (biotherapy) and/or alternative therapies.

In some embodiments antibiotics can be used in combination with the pharmaceutical composition to treat and/or prevent an infectious disease. Such antibiotics include, but are not limited to, amikacin, aminoglycosides (e.g., gentamycin), amoxicillin, amphotericin B, ampicillin, antimonials, atovaquone sodium stibogluconate, azithromycin, capreomycin, cefotaxime, cefoxitin, ceftriaxone, chloramphenicol, clarithromycin, clindamycin, clofazimine, cycloserine, dapsone, doxycycline, ethambutol, ethionamide, fluconazole, fluoroquinolones, isoniazid, itraconazole, kanamycin, ketoconazole, minocycline, ofloxacin), para-aminosalicylic acid, pentamidine, polymixin definsins, prothionamide, pyrazinamide, pyrimethamine sulfadiazine, quinolones (e.g., ciprofloxacin), rifabutin, rifampin, sparfloxacin, streptomycin, sulfonamides, tetracyclines, thiacetazone, trimethaprim-sulfamethoxazole, viomycin or combinations thereof.

More generally, such an agent would be provided in a combined amount with the expression vector effective to kill or inhibit proliferation of a cancer cell and/or microorganism. This process may involve contacting the cell(s) with an agent(s) and the pharmaceutical composition at the same time or within a period of time wherein separate administration of the pharmaceutical composition and an agent to a cell, tissue or organism produces a desired therapeutic benefit. This may be achieved by contacting the cell, tissue or organism with a single composition or pharmacological formulation that includes both the pharmaceutical composition and one or more agents, or by contacting the cell with two or more distinct compositions or formulations, wherein one composition includes the pharmaceutical composition and the other includes one or more agents.

The terms “contacted” and “exposed,” when applied to a cell, tissue or organism, are used herein to describe the process by which the pharmaceutical composition and/or another agent, such as for example a chemotherapeutic or radiotherapeutic agent, are delivered to a target cell, tissue or organism or are placed in direct juxtaposition with the target cell, tissue or organism. To achieve cell killing or stasis, the pharmaceutical composition and/or additional agent(s) are delivered to one or more cells in a combined amount effective to kill the cell(s) or prevent them from dividing. In some embodiments, the chemotherapeutic agent is selected from the group consisting of carboplatin, estramustine phosphate (Emcyt), and thalidomide. In some embodiments, the chemotherapeutic agent is a taxane. The taxane may be, for example, selected from the group consisting of docetaxel (Taxotere), paclitaxel, and cabazitaxel. In some embodiments, the taxane is docetaxel. In some embodiments, the chemotherapeutic agent is administered at the same time or within one week after the administration of the modified cell or nucleic acid. In other embodiments, the chemotherapeutic agent is administered from 1 to 4 weeks or from 1 week to 1 month, 1 week to 2 months, 1 week to 3 months, 1 week to 6 months, 1 week to 9 months, or 1 week to 12 months after the administration of the modified cell or nucleic acid. In some embodiments, the chemotherapeutic agent is administered at least 1 month before administering the cell or nucleic acid.

The administration of the pharmaceutical composition may precede, be concurrent with and/or follow the other agent(s) by intervals ranging from minutes to weeks. In embodiments where the pharmaceutical composition and other agent(s) are applied separately to a cell, tissue or organism, one would generally ensure that a significant period of time did not expire between the times of each delivery, such that the pharmaceutical composition and agent(s) would still be able to exert an advantageously combined effect on the cell, tissue or organism. For example, in such instances, it is contemplated that one may contact the cell, tissue or organism with two, three, four or more modalities substantially simultaneously (i.e., within less than about a minute) with the pharmaceutical composition. In other aspects, one or more agents may be administered within from substantially simultaneously, about 1 minute, to about 24 hours to about 7 days to about 1 to about 8 weeks or more, and any range derivable therein, prior to and/or after administering the expression vector. Yet further, various combination regimens of the pharmaceutical composition presented herein and one or more agents may be employed.

In some embodiments, the chemotherapeutic agent may be a lymphodepleting chemotherapeutic. In other examples, the chemotherapeutic agent may be Taxotere (docetaxel), or another taxane, such as, for example, cabazitaxel. The chemotherapeutic may be administered before, during, or after treatment with the cells and inducer. For example, the chemotherapeutic may be administered about 1 year, 11, 10, 9, 8, 7, 6, 5, or 4 months, or 18, 17, 16, 15, 14, 13, 12, 11, 10, 9, 8, 7, 6, 5, 4, 3, 2, weeks or 1 week prior to administering the first dose of activated nucleic acid. Or, for example, the chemotherapeutic may be administered about 1 week or 2, 3, 4, 5, 6, 7, 8, 9, 10, 11, 12, 13, 14, 15, 16, 17, or 18 weeks or 4, 5, 6, 7, 8, 9, 10, or 11 months or 1 year after administering the first dose of cells or inducer.

Administration of a chemotherapeutic agent may comprise the administration of more than one chemotherapeutic agent. For example, cisplatin may be administered in addition to Taxotere or other taxane, such as, for example, cabazitaxel.

In some embodiments, the invention provides for combination therapies comprising the modified cell population described herein with cytokines or chemokines neutralizing agent, e.g. a neutralizing antibody. In some embodiments, the invention provides for combination therapies comprising the modified cell population described herein and a TNFα neutralizing agent, e.g., an anti-TNFα antibody.

Indication for Adjusting or Maintaining Subsequent Drug Dose

An indication for adjusting or maintaining a subsequent drug dose can be based on the presence or absence of a biomarker. For example, when (i) low sensitivity determinations of biomarker levels are available, (ii) biomarker levels shift sharply in response to a drug, (iii) low levels or high levels of biomarker are present, and/or (iv) a drug is not appreciably toxic at levels of administration, presence or absence of a biomarker can be sufficient for generating an indication of adjusting or maintaining a subsequent drug dose.

An indication for adjusting or maintaining a subsequent drug dose often is based on the amount or level of a biomarker (e.g., PSCA). An amount of a biomarker can be a mean, median, nominal, range, interval, maximum, minimum, or relative amount, in some embodiments. An amount of a biomarker can be expressed with or without a measurement error window in certain embodiments.

An amount of a biomarker in some embodiments can be expressed as a biomarker concentration, biomarker weight per unit weight, biomarker weight per unit volume, biomarker moles, biomarker moles per unit volume, biomarker moles per unit weight, biomarker weight per unit cells, biomarker volume per unit cells, biomarker moles per unit cells and the like. Weight can be expressed as femtograms, picograms, nanograms, micrograms, milligrams and grams, for example. Volume can be expressed as femtoliters, picoliters, nanoliters, microliters, milliliters and liters, for example. Moles can be expressed in picomoles, nanomoles, micromoles, millimoles and moles, for example. In some embodiments, unit weight can be weight of subject or weight of sample from subject, unit volume can be volume of sample from the subject (e.g., blood sample volume) and unit cells can be per one cell or per a certain number of cells (e.g., micrograms of biomarker per 1000 cells). In some embodiments, an amount of biomarker determined from one tissue or fluid can be correlated to an amount of biomarker in another fluid or tissue, as known in the art.

An indication for adjusting or maintaining a subsequent drug dose often is generated by comparing a determined level of biomarker in a subject to a predetermined level of biomarker. A predetermined level of biomarker sometimes is linked to a therapeutic or efficacious amount of drug in a subject, sometimes is linked to a toxic level of a drug, sometimes is linked to presence of a condition, sometimes is linked to a treatment midpoint and sometimes is linked to a treatment endpoint, in certain embodiments. A predetermined level of a biomarker sometimes includes time as an element, and in some embodiments, a threshold is a time-dependent signature.

Some treatment methods comprise (i) administering a drug to a subject in one or more administrations (e.g., 1, 2, 3, 4, 5, 6, 7, 8, 9 or 10 doses), (ii) determining the presence, absence or amount of a biomarker (e.g., PSCA) in or from the subject after (i), (iii) providing an indication of increasing, decreasing or maintaining a subsequent dose of the drug for administration to the subject, and (iv) optionally administering the subsequent dose to the subject, where the subsequent dose is increased, decreased or maintained relative to the earlier dose(s) in (i). In some embodiments, presence, absence or amount of a biomarker is determined after each dose of drug has been administered to the subject, and sometimes presence, absence or amount of a biomarker is not determined after each dose of the drug has been administered (e.g., a biomarker is assessed after one or more of the first, second, third, fourth, fifth, sixth, seventh, eighth, ninth or tenth dose, but not assessed every time after each dose is administered).

An indication for adjusting a subsequent drug dose can be considered a need to increase or a need to decrease a subsequent drug dose. An indication for adjusting or maintaining a subsequent drug dose can be considered by a clinician, and the clinician may act on the indication in certain embodiments. In some embodiments, a clinician may opt not to act on an indication. Thus, a clinician can opt to adjust or not adjust a subsequent drug dose based on the indication provided.

An indication of adjusting or maintaining a subsequent drug dose, and/or the subsequent drug dosage, can be provided in any convenient manner. An indication may be provided in tabular form (e.g., in a physical or electronic medium) in some embodiments. For example, a biomarker threshold may be provided in a table, and a clinician may compare the presence, absence or amount of the biomarker determined for a subject to the threshold. The clinician then can identify from the table an indication for subsequent drug dose. In certain embodiments, an indication can be presented (e.g., displayed) by a computer after the presence, absence or amount of a biomarker is provided to computer (e.g., entered into memory on the computer). For example, presence, absence or amount of a biomarker determined for a subject can be provided to a computer (e.g., entered into computer memory by a user or transmitted to a computer via a remote device in a computer network), and software in the computer can generate an indication for adjusting or maintaining a subsequent drug dose, and/or provide the subsequent drug dose amount. A subsequent dose can be determined based on certain factors other than biomarker presence, absence or amount, such as weight of the subject, one or more metabolite levels for the subject (e.g., metabolite levels pertaining to liver function) and the like, for example.

Once a subsequent dose is determined based on the indication, a clinician may administer the subsequent dose or provide instructions to adjust the dose to another person or entity. The term “clinician” as used herein refers to a decision maker, and a clinician is a medical professional in certain embodiments. A decision maker can be a computer or a displayed computer program output in some embodiments, and a health service provider may act on the indication or subsequent drug dose displayed by the computer. A decision maker may administer the subsequent dose directly (e.g., infuse the subsequent dose into the subject) or remotely (e.g., pump parameters may be changed remotely by a decision maker).

A subject can be prescreened to determine whether or not the presence, absence or amount of a particular biomarker (e.g., PSCA) may be determined. Non-limiting examples of prescreens include identifying the presence or absence of a genetic marker (e.g., polymorphism, particular nucleotide sequence); identifying the presence, absence or amount of a particular metabolite. A prescreen result can be used by a clinician in combination with the presence, absence or amount of a biomarker to determine whether a subsequent drug dose may be adjusted or maintained.

### EXAMPLES

The examples set forth below illustrate certain embodiments and do not limit the technology.

### Example 1: PSCA-Specific CAR-T Cell Clinical Study

The PSCA-specific CAR-T cells of this example are PSCA-directed, genetically modified, autologous product candidate that binds to PSCA-expressing cells. PSCA-specific CAR-T cells target PSCA with a first-generation CAR construct containing a traditional CD3ζ cytoplasmic signaling domain together with a single-chain variable region domain (scFV) of a humanized PSCA antibody. In addition to the anti-PSCA CAR, the PSCA-specific CAR-T cells were engineered to express an inducible dual co-stimulatory domain comprised of two FK binding proteins (FKBP) in-frame with the signaling domains from MyD88 and CD40 (inducible MyD88/CD40 or iMC).

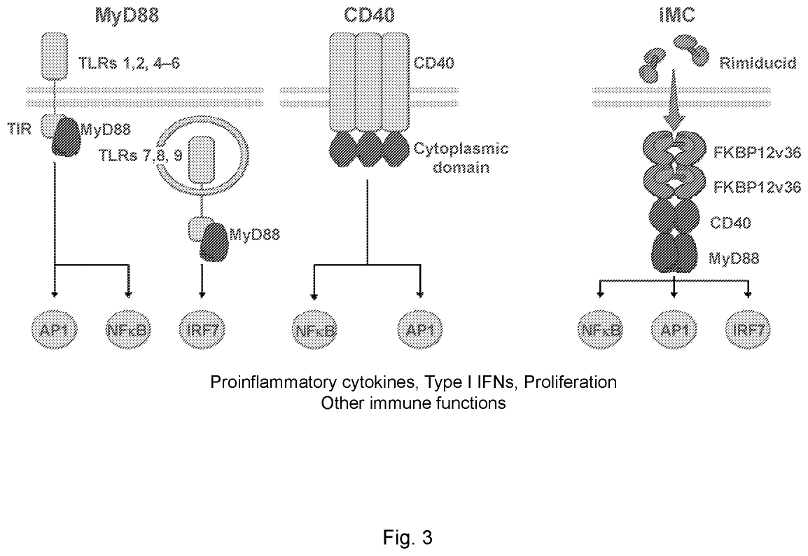

iMC functions as a molecular switch to enhance and control the activation and proliferation of T cells in the presence of the small molecule dimerizer rimiducid (FIG. 3). The tandem FKBP domains provide a ligand dimerization scaffold, which induces iMC in a rimiducid-dependent manner to initiate downstream signaling pathways that upregulate proinflammatory cytokines and type I interferons as well as promote cell proliferation and survival.

The inducible MyD88/CD40 PSCA CAR retroviral construct contains, in the 5′ to 3′ direction, a polynucleotide encoding:

(1) a fusion protein containing a truncated human MyD88 polypeptide (the amino terminal 172 amino acids of MyD88 containing the DD domain and intermediary domain; SEQ ID NO: 21 encoded by SEQ ID NO: 22) fused to a portion of a human CD40 polypeptide (the carboxy terminal 62 amino acids, i.e., amino acids 216-277 of CD40; SEQ ID NO: 23 encoded by SEQ ID NO: 24), (the entire fusion protein is termed MC), which is fused to two copies of a mutant human FKBP12 protein (FKBP12(F36V)) also known as FKBP12v36, FV36, FKBPv, or Fv; SEQ ID NOs: 41 and 43, encoded by SEQ ID NOs: 42 and 44) in which the phenylalanine at amino acid position 36 (or 37 if the initial methionine of the protein is counted) is substituted by a valine (the entire fusion protein may be named iMC),

(2) a T2A polypeptide (SEQ ID NO: 5 encoded by SEQ ID NO: 6), and

(3) a first generation CAR polypeptide including, in the 5′ to 3′ direction, an scFv sequence (comprising a membrane signal peptide (SEQ ID NO: 7 encoded by SEQ ID NO: 8) fused to light and heavy chain variable regions of an anti-PSCA monoclonal antibody with an intervening 8-amino acid flexible glycine-serine linker, i.e., flex peptide (SEQ ID NO: 9 encoded by SEQ ID NO: 10) between the chains) fused to a human CD34 epitope polypeptide (amino acids 30-45 of CD34; SEQ ID NO: 11 encoded by SEQ ID NO: 12) which is fused to an alpha stalk region of human CD8 (amino acids 141-182 of CD8; SEQ ID NO: 13 encoded by SEQ ID NO: 14) which is fused to the transmembrane domain of human CD8 (amino acids 183-219 of CD8; SEQ ID NO: 15 encoded by SEQ ID NO: 16) which is fused to a portion of human CD3ζ (amino acids 83-194 of CD3ζ isoform X2; SEQ ID NO: 17 encoded by SEQ ID NO: 18).

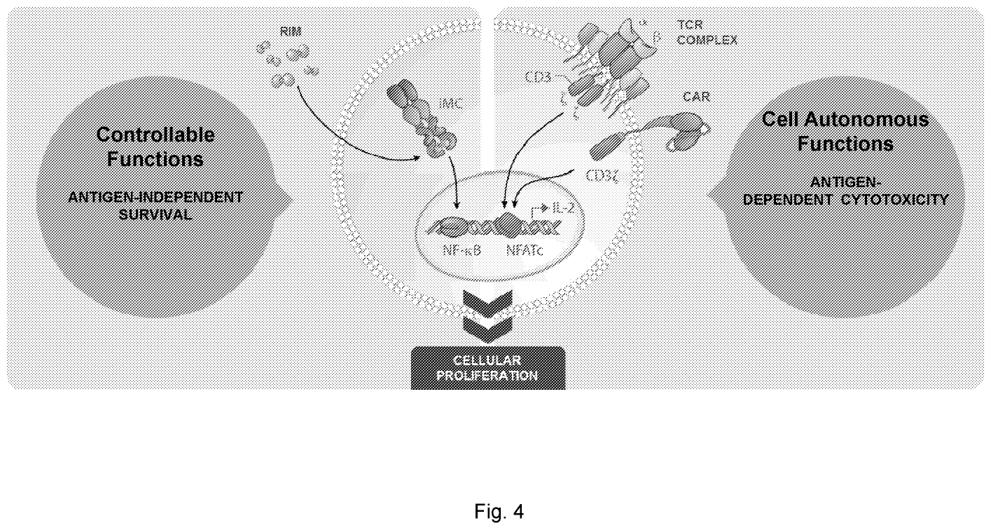

Inducible MyD88/CD40 PSCA CAR-T cells were rationally designed to proliferate in response two distinct signals (FIG. 4): 1) antigen-specific activation through PSCA recognition and CD3ζ signaling domain (Signal 1); and, 2) rimiducid-dependent costimulation through iMC (Signal 2). This design may optimize inducible MyD88/CD40 PSCA CAR-T cells for both antigen-directed as well as antigen-independent T cell activation, proliferation, and persistence and thereby may afford enhanced clinical activity relative to traditional CARs for the treatment of solid tumor malignancies (Signal 3; FIG. 4).

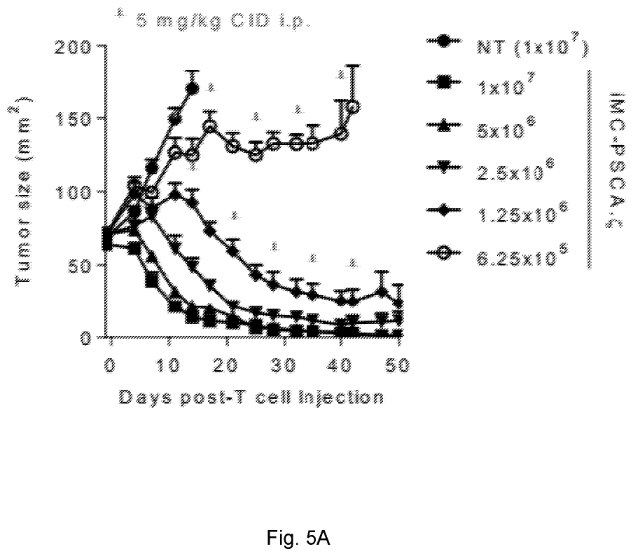

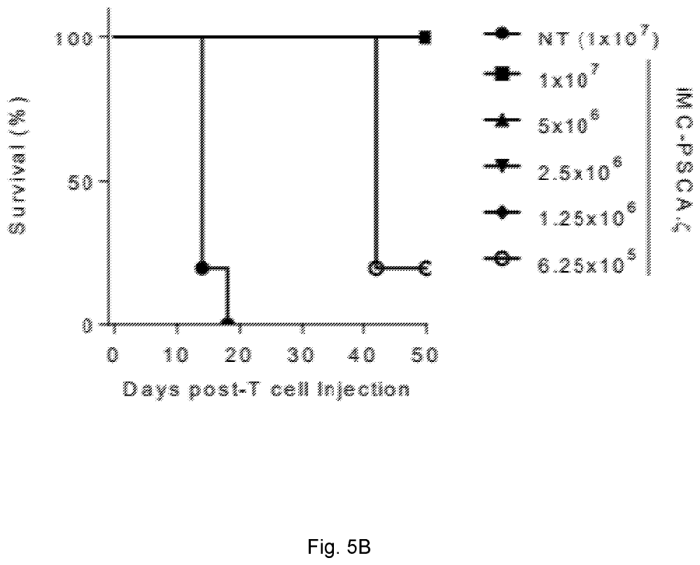

To determine if a dose-response relationship exists for antigen-dependent cytotoxicity as well as to assess whether iMC may influence this, tumor-bearing NSG mice were treated with single, ascending doses of inducible MyD88/CD40 PSCA CAR-T cells (0.625×10⁶ cells, 1.25×10⁶ cells, 2.5×10⁶ cells, 5.0×10⁶ cells, and 10.0×10⁶ cells) were administered by intravenous injection on Day 0 (FIG. 5A, FIG. 5B). If tumor measurements showed growth on two consecutive measurements following T cell injection, weekly intraperitoneal rimiducid 5 mg/kg was initiated. By Day 14, doses ≥1.25×10⁶ cells resulted in rapid control and eventual elimination of HPAC tumors. Doses ≤1.25×10⁶ cells were insufficient to reduce tumor volume in the absence of rimiducid. Following iMC activation, however, tumor growth was either stabilized or reduced in mice receiving 0.625×10⁶ or 1.25×10⁶ cells, respectively. Together, these data not only support that activation of iMC with rimiducid in HPAC tumor-bearing mice can facilitate antitumor function but also define 1.0×10⁶ cells as a minimum dose level to affect tumor growth in vivo. Furthermore, all inducible MyD88/CD40 PSCA CAR-T cell-treated mice maintained or gained weight over the course of the experiment regardless of T cell dose level with or without rimiducid treatment, suggesting that therapy was well-tolerated with no signs of overt toxicity.

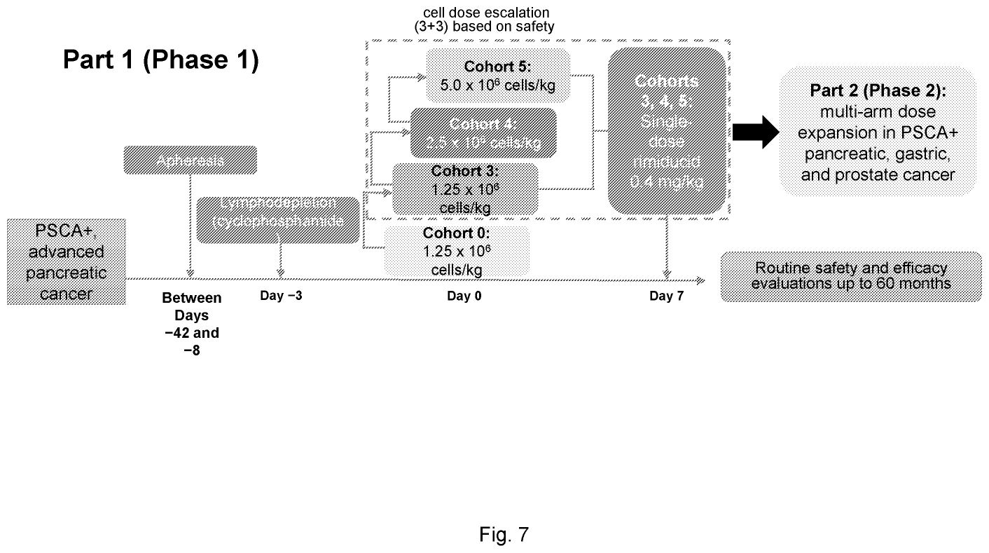

To determine the feasibility, safety, and clinical activity of PSCA-specific CAR-T cells, administered with rimiducid to subjects with previously treated, PSCA-positive advanced solid tumors (pancreatic, gastric/GEJ, prostate cancers), the study depicted in FIG. 7 was designed.

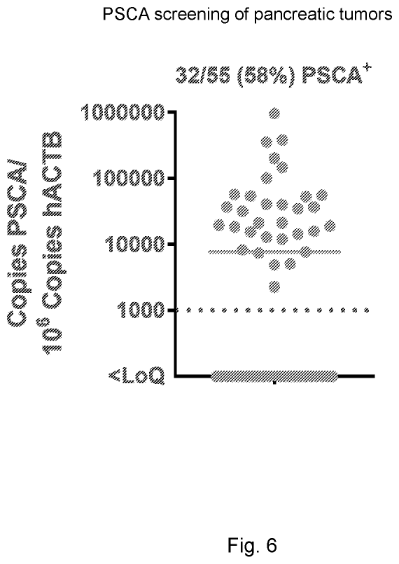

FIG. 6 shows PSCA screening of pancreatic tumors.

Part 1 (Phase 1): Cell dose escalation to identify the maximum dose of inducible MyD88/CD40 PSCA CAR-T cells (escalating doses from 1.25×10⁶ cells/kg up to 5.0×10⁶ cells/kg) administered with rimiducid (fixed single-dose at 0.4 mg/kg).

Parts 2 (Phase 2): Indication-specific dose expansion to assess the safety, pharmacodynamics (including inducible MyD88/CD40 PSCA CAR-T cell persistence), and clinical activity at a dose identified in Part 1 in various PSCA-expressing solid tumors.

The initial starting dose for inducible MyD88/CD40 PSCA CAR-T cells was 1.25×10⁶ cells/kg. Rimiducid was administered at a fixed dose of 0.4 mg/kg.

Cohort 0 was completed as follows: three subjects were enrolled and received PSCA CAR-T cells (1.25×10⁶ cells/kg) on Day 0 in the absence of subsequent rimiducid. No dose limiting toxicity events were observed and there were no adverse events (AE), elevated cytokine levels, or other signs of toxicity associated with PSCA CAR-T cell infusion. Quantitative polymerase chain reaction (qPCR) analysis of peripheral blood samples revealed PSCA CAR-T cell counts increasing approximately between Days 0 to 15, with maximum expansion observed from Days 5 to 15, followed by an eventual decrease below the level of assay detection by Day 21. Based on the favorable safety profile and early clearance of PSCA CAR-T cells demonstrated in Cohort 0, the addition of a fixed, single dose of rimiducid (0.4 mg/kg) on Day 7 to the investigational treatment regimen was performed.

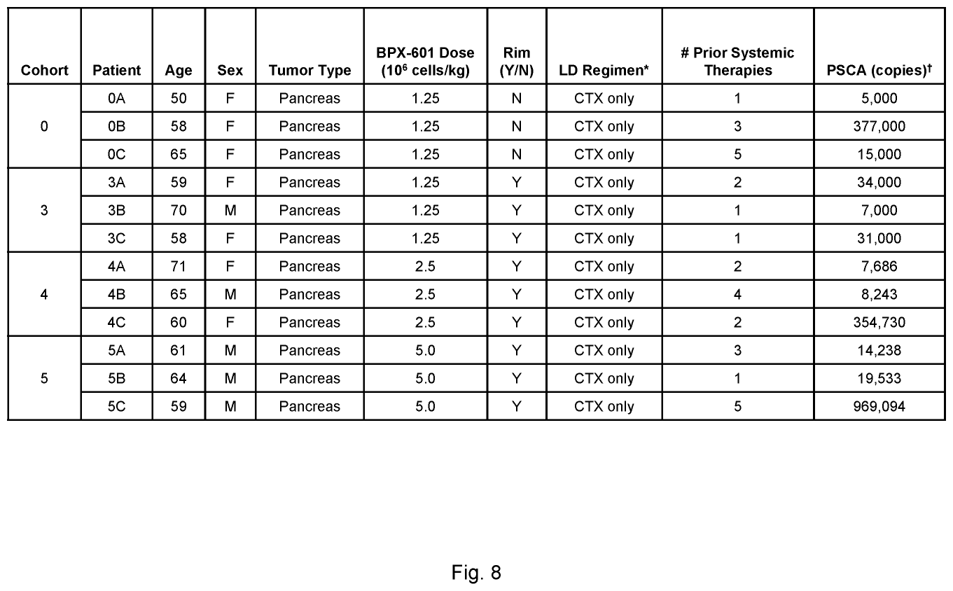

Cohort 3 (1.25×10⁶ cells/kg) was the initial starting cohort for cell dose escalation decisions in Part 1 when PSCA CAR-T cells are administered with rimiducid. Cohorts of 3 subjects were treated with escalating doses of PSCA CAR-T cells on Day 0 with a fixed, single dose of rimiducid (0.4 mg/kg) on Day 7. The doses of PSCA CAR-T cells are detailed in FIG. 8.

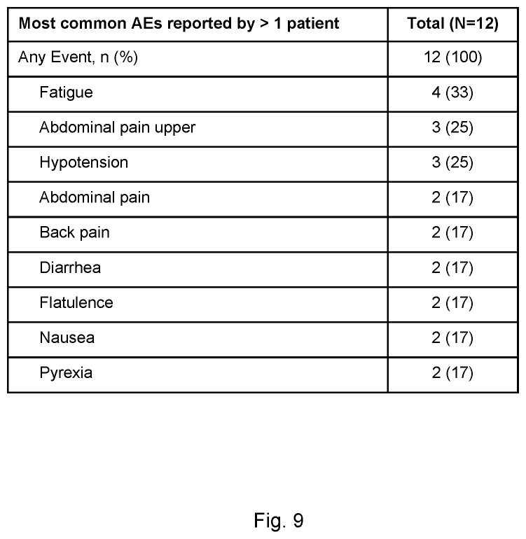

Results are presented in FIGS. 9-14. A safety summary is provided in FIG. 9. No DLTs, neurotoxicity, or events of cytokine release syndrome were observed. Pyrexia was the only treatment-related AE reported by reported by 2 or more patients. Both events resolved within 24-36 hours with supportive care. Specifically, one patient in Cohort 4 experienced Grade 1 pyrexia following infusion of inducible MyD88/CD40 PSCA CAR-T cells on Day 0; the event recovered within 24 hours with paracetamol. One patient in Cohort 5 experienced Grade 2 pyrexia following infusion of inducible MyD88/CD40 PSCA CAR-T cells on Day 0. Although the event recovered within 36 hours with paracetamol, this was reported as an SAE as the fever prolonged the patient's planned infusion-related admission. No other treatment-related SAEs were reported. Both pyrexia events were associated with time-matched, transient increases in peripheral cytokines. The Grade 1 event was accompanied by moderate hypotension that recovered within 2 hours with IV fluids; the Grade 2 event was isolated and occurred in the absence of other systemic clinical symptoms. 6 patients reported 14 SAEs, all but one (Grade 2 pyrexia) were related to the disease under study.

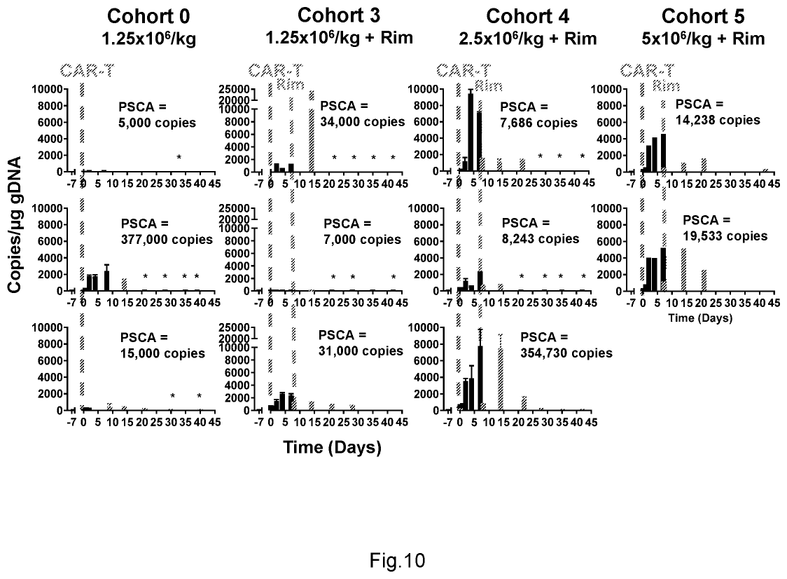

Inducible MyD88/CD40 PSCA CAR-T cell expansion and persistence is shown in FIG. 10. Limited evidence of lymphodepletion (LD) with cyclophosphamide (CTX)-only regimen (79%±25% of cells remained) was observed. Rapid cell expansion by Day 4 was observed, but no persistence without rimiducid. Rapid cell engraftment by Day 4 was observed in the majority of patients despite an ineffective lymphodepletion in all but 1 subject. The mean percentage of lymphocytes remaining after lymphodepletion with cyclophosphamide was 79% (SD 25%). Of 8 patients that received a single dose of rimiducid, 4 had cell expansion at least 3- and up to 20-fold within 7 days and 3 had cell persistence >3 weeks following rimiducid infusion. Cell persistence >4 months was observed in one patient who also had the highest PSCA expression at screening. In Cohort 0, limited peripheral expansion was observed when inducible MyD88/CD40 PSCA CAR-T cell were infused alone.

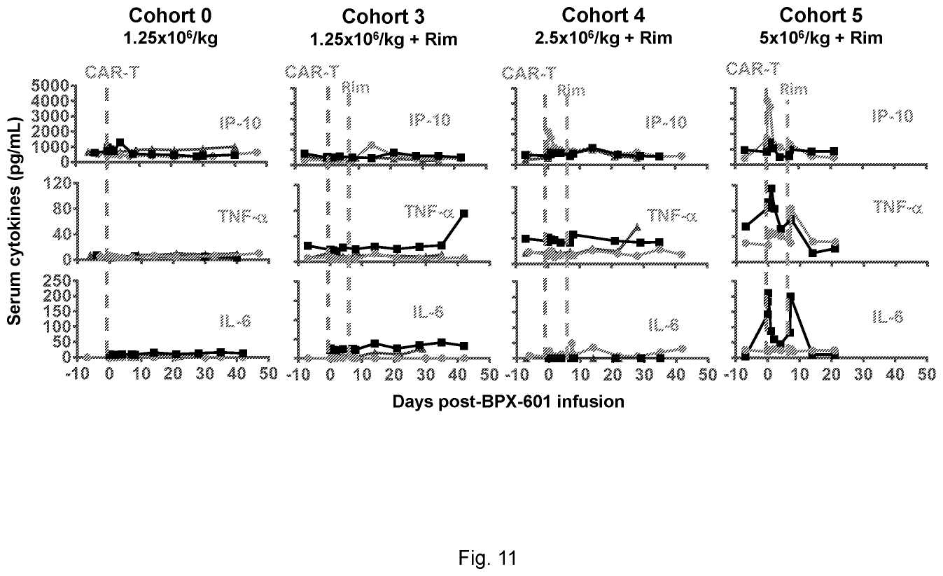

Peripheral cytokine profiles over time are shown in FIG. 11. Of the 8 patients that received rimiducid, all had elevated cytokines (IP-10, TNF-α) correlated with cell expansion. Increased serum IP-10 is indicative of prior IFNγ production and together with increased serum TNF-α supports T cell activation in response to activation of iMC by rimiducid. Increased cell dose is associated with overall increased serum cytokine and chemokine levels. Increased chemokine levels were observed in many patients after rim infusion including MCP-1, MIP-1α and MIP-1β, which are produced by activated lymphocytes and macrophages and have important chemotactic and pro-inflammatory function. A reduction in peripheral lymphocytes (associated with an increase in serum IL-7 and IL-15 levels) was observed in 1 patient where increased IL-7 and IL-15 levels after lymphodepletion were associated with a reduction in the peripheral lymphocyte count to 31% of the starting level. Overall, lymphocytes were reduced to an average of 78.7+/−25% of the starting level.

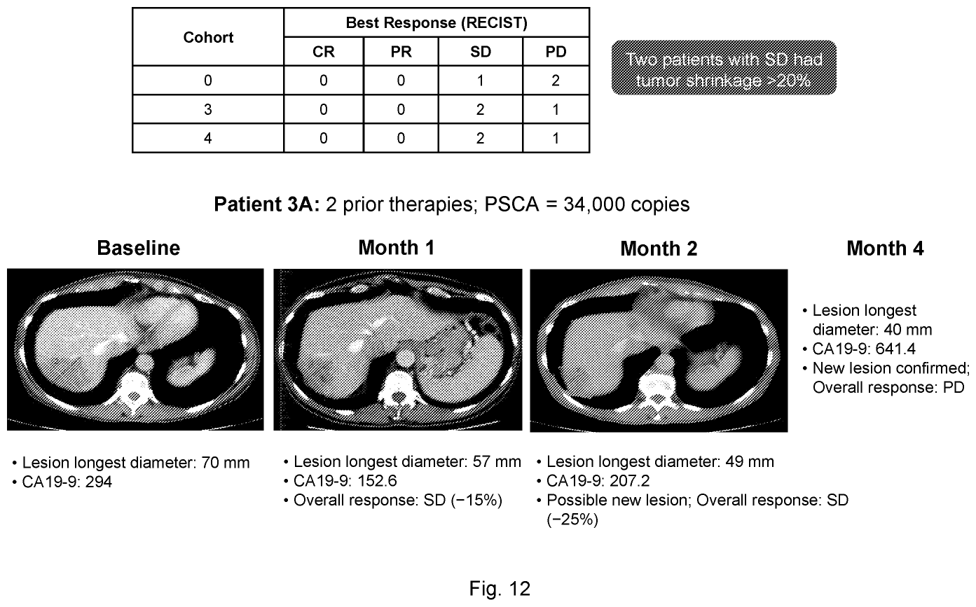

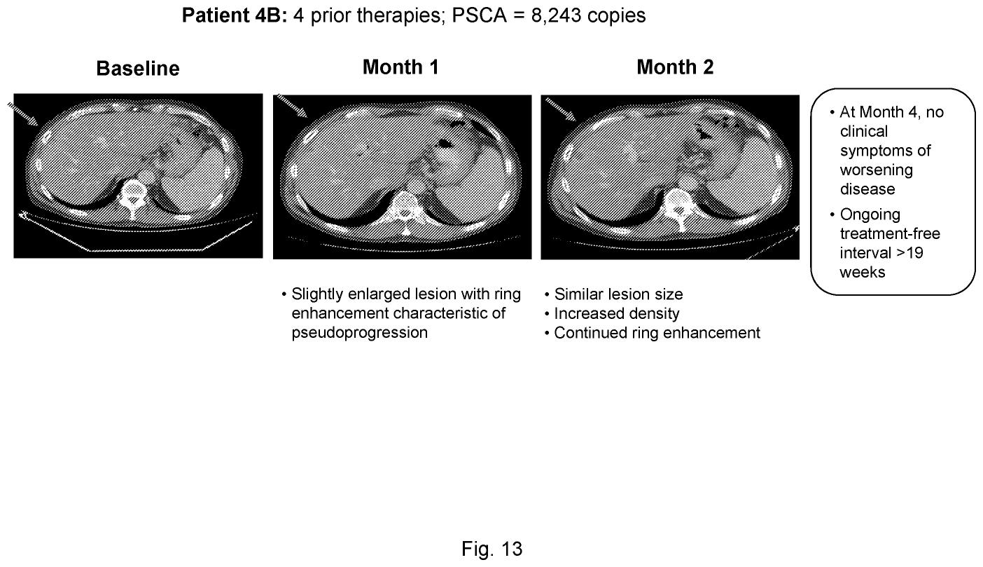

Evidence of anti-tumor activity in patients treated with inducible MyD88/CD40 PSCA CAR-T cells is shown in FIG. 12 and FIG. 13. Scans were performed at week 4 (month 1), week 8 (month 2), and every 8 weeks (every 2 months) thereafter until progression. Two patients had tumor shrinkage >20%.

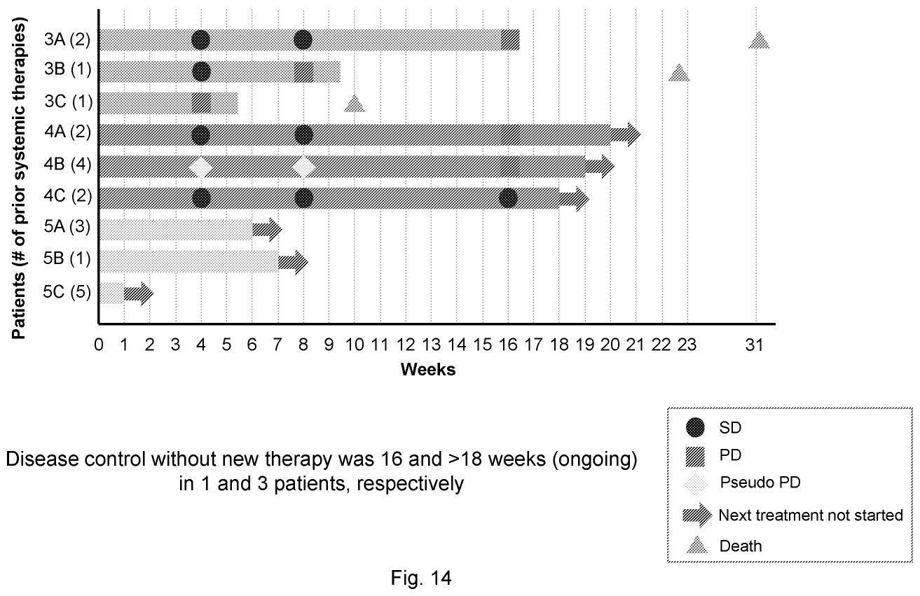

FIG. 14 show time to next treatment after PSCA CAR-T cells administration.

Thus, together these results show that administration of PSCA CAR-T cell with single-dose rimiducid was well tolerated. No observed cytokine release syndrome or neurotoxicity of any grade. Most frequent AEs were consistent with those experienced by advanced cancer patients undergoing cytotoxic chemotherapy or other cancer immunotherapies.

Treatment-related AEs were limited, mild to moderate in intensity, and resolved with supportive care. In addition, despite lack of lymphodepletion with cyclophosphamide alone, PSCA CAR-T cells displayed enhanced expansion and prolonged persistence in some patients. Thus, evidence of biological activity/disease control have been observed in this heavily pre-treated patient population

Methods

Manufacturing Process Description: PSCA CAR-T cells are prepared from a patient's peripheral blood mononuclear cells (PBMCs), which are obtained via a standard apheresis procedure described below. PBMCs are enriched for T cells which are then washed and expanded in cell culture. Once a target number of cells are available, the expanded T cells are transduced with a replication incompetent retroviral vector containing the anti-PSCA CAR and rimiducid-inducible iMC transgenes. The transduced T cells are formulated into a suspension, and cryopreserved. The final PSCA CAR-T cells product must pass release testing, including a sterility test, before shipping as a frozen suspension in a patient-specific infusion bag.

Packaging and Formulation: PSCA CAR-T cells are cryopreserved in 10-15 mL freezing medium and are stored frozen in cryostorage bags in the vapor phase of liquid nitrogen.

Apheresis

Subjects underwent peripheral blood mononuclear cell (PBMC) collection according to local institutional standards. Prior to apheresis, the subject's white blood cell count and differential measured and recorded. Results must demonstrate that the subject has adequate bone marrow function that is transfusion independent and without growth factor support:
White blood cell count ≥2×10³/μL with absolute lymphocyte count ≥1×10²/μL
Absolute neutrophil count ≥1×10³/μL without granulocyte colony stimulating factor support
Platelets ≥100×10³/μL
Hemoglobin ≥9 g/dL or as per institutional guidelines for apheresis

1-2 blood volumes were collected and processed using standardized continuous flow centrifugation, as per institutional standard procedures. The volume processed, and the duration of apheresis was recorded. During and after the apheresis procedure, subjects underwent infectious disease monitoring per established regulatory guidelines. Evaluation prior to apheresis, venous access, monitoring and treatment of apheresis complications were conducted according to institutional guidelines.

Lymphodepletion Prior to PSCA CAR T Cell Infusion

A lymphodepleting chemotherapy regimen of cyclophosphamide 500 mg/m² intravenously and fludarabine 30 mg/m² intravenously was administering on the fifth, fourth, and third day before infusion of PSCA CAR T cells, unless the combination was not tolerated. If it was determined that combination-based lymphodepletion presented an unfavorable safety risk to a subject, then lymphodepletion proceeded with cyclophosphamide alone.

### Example 2: Nucleic Acid and Amino Acid Sequences

TABLE 1
amino acid sequences

| SEQ ID | | |
|---|---|---|
| NO: | PROTEIN | AMINO ACID SEQUENCE |
| 1 | Fᵥ | GVQVETISPGDGRTFPKRGQTCVVHYTGMLEDGKKVDSSRDRNKPFKFMLGKQEVIRGWEEG |
| | Human | VAQMSVGQRAKLTISPDYAYGATGHPGIIPPHATLVFDVELLKLE |
| | FKBP12v36 | |
| 3 | 8-amino acid | SGGGSGVD |
| | linker | |
| 5 | T2A | EGRGSLLTCGDVEENPGP |
| | polypeptide | |
| 7 | Signal | MEFGLSWLFLVAILKGVQCSR |
| | peptide | |
| 9 | Glycine- | GGGSGGGG |
| | serine linker | |
| 11 | Human CD34 | ELPTQGTFSNVSTNVS |
| | epitope | |
| 13 | Human CD8 | PAPRPPTPAPTIASQPLSLRPEACRPAAGGAVHTRGLDFACD |
| | alpha stalk | |
| 15 | Human CD8 | IYIWAPLAGTCGVLLLSLVITLYCNHRNRRRVCKCPR |
| | trans- | |
| | membrane | |
| | region | |
| | between | |
| | FKBP12-1 | |
| | and FKBP12- | |
| | 2 in pM004 | |
| 17 | Portion of | RVKFSRSADAPAYQQGQNQLYNELNLGRREEYDVLDKRRGRDPEMGGKPRRKNPQEGLYNE |
| | human CD3ζ | LQKDKMAEAYSEIGMKGERRRGKGHDGLYQGLSTATKDTYDALHMQALPPR |
| | encoded by | |
| | poly- | |
| | nucleotide in | |
| | vector | |
| 19 | P2A | ATNFSLLKQAGDVEENPGP |
| | polypeptide | |
| 21 | Portion of | MAAGGPGAGSAAPVSSTSSLPLAALNMRVRRRLSLFLNVRTQVAADVVTALAEEMDFEYLEIRQ |
| | human | LETQADPTGRLLDAWQGRPGASVGRLLDLLTKLGRDDVLLELGPSIEEDCQKYILKQQQEEAEK |
| | MyD88 | PLQVAAVDSSVPRTAELAGITTLDDPLGHMPERFDAFICYCPSDI |
| | polypeptide | |
| | encoded by | |
| | poly- | |
| | nucleotide in | |
| | vector | |
| 23 | Portion of | KKVAKKPTNKAPHPKQEPQEINFPDDLPGSNTAAPVQETLHGCQPVTQEDGKESRISVQERQ |
| | human CD40 | |
| | polypeptide | |
| | encoded by | |
| | poly- | |
| | nucleotide in | |
| | vector | |
| 25 | MLEMLE | MLEMLE |
| | linker | |
| | encoded 5′ of | |
| | FKBP12v36 | |
| | by poly- | |
| | nucleotide in | |
| | vector | |
| 27 | Human | MGVQVETISPGDGRTFPKRGQTCVVHYTGMLEDGKKFDSSRDRNKPFKFMLGKQEVIRGWEE |
| | FKBP12 | GVAQMSVGQRAKLTISPDYAYGATGHPGIIPPHATLVFDVELLKLE |
| | (GenBank no | |
| | AAA58476) | |
| 29 | Human | MAAGGPGAGSAAPVSSTSSLPLAALNMRVRRRLSLFLNVRTQVAADVVTALAEEMDFEYLEIRQ |
| | MyD88 | LETQADPTGRLLDAWQGRPGASVGRLLELLTKLGRDDVLLELGPSIEEDCQKYILKQQQEEAEK |
| | (Genbank no. | PLQVAAVDSSVPRTAELAGITTLDDPLGHMPERFDAFICYCPSDIQFVQEMIRQLEQTNYRLKLC |
| | AAC50954) | VSDRDVLPGTCVWSIASELIEKRCRRMVVVVSDDYLQSKECDFQTKFALSLSPGAHQKRLIPIKY |
| | | KAMKKEFPSILRFITVCDYTNPCTKSWFWTRLAKALSLP |
| 31 | Human CD40 | MVRLPLQCVLWGCLLTAVHPEPPTACREKQYLINSQCCSLCQPGQKLVSDCTEFTETECLPCG |
| | (Genbank no. | ESEFLDTWNRETHCHQHKYCDPNLGLRVQQKGTSETDTICTCEEGWHCTSEACESCVLHRSC |
| | AAH12419) | SPGFGVKQIATGVSDTICEPCPVGFFSNVSSAFEKCHPWTSCETKDLVVQQAGTNKTDVVCGP |
| | | QDRLRALVVIPIIFGILFAILLVLVFIKKVAKKPTNKAPHPKQEPQEINFPDDLPGSNTAAPVQETLH |
| | | GCQPVTQEDGKESRISVQERQ |
| 33 | Human CD3ζ | MKWKALFTAAILQAQLPITASSLPHPTQQSPEKKVLGPGGCTCRHNRFCNEAQSFGLLDPKLCY |
| | (GenBank no. | LLDGILFIYGVILTALFLRVKFSRSADAPAYQQGQNQLYNELNLGRREEYDVLDKRRGRDPEMG |
| | XP_0168582 | GKPRRKNPQEGLYNELQKDKMAEAYSEIGMKGERRRGKGHDGLYQGLSTATKDTYDALHMQA |
| | 90) | LPPR |
| 35 | MLE linker | MLE |
| 37 | Linker | GGGGSGGGGSGGGGS |
| 39 | ΔCD19 | MPPPRLLFFLLFLTPMEVRPEEPLVVKVEEGDNAVLQCLKGTSDGPTQQLTWSRESPLKPFLKL |
| | marker | SLGLPGLGIHMRPLAIWLFIFNVSQQMGGFYLCQPGPPSEKAWQPGWTVNVEGSGELFRWNV |
| | polypeptide | SDLGGLGCGLKNRSSEGPSSPSGKLMSPKLYVWAKDRPEIWEGEPPCLPPRDSLNQSLSQDL |
| | | TMAPGSTLWLSCGVPPDSVSRGPLSWTHVHPKGPKSLLSLELKDDRPARDMWVMETGLLLPR |
| | | ATAQDAGKYYCHRGNLTMSFHLEITARPVLWHWLLRTGGWKVSAVTLAYLIFCLCSLVGILHLQ |
| | | RALVLRRKRKRMTDPTRRF |
| 41 | Fv | GVQVETISPGDGRTFPKRGQTCVVHYTGMLEDGKKVDSSRDRNKPFKFMLGKQEVIRGWEEG |
| | | VAQMSVGQRAKLTISPDYAYGATGHPGIIPPHATLVFDVELLKLE |
| 43 | Fv′ | GVQVETISPGDGRTFPKRGQTCVVHYTGMLEDGKKVDSSRDRNKPFKFMLGKQEVIRGWEEG |
| | | VAQMSVGQRAKLTISPDYAYGATGHPGIIPPHATLVFDVELLKL |
| 45 | FKBP12 | GVQVETISPGDGRTFPKRGQTCVVHYTGMLEDGKKFDSSRDRNKPFKFMLGKQEVIRGWEEG |
| | Wild type | VAQMSVGQRAKLTISPDYAYGATGHPGIIPPHATLVFDVELLKLE |
| 47 | MyD88 | MAAGGPGAGSAAPVSSTSSLPLAALNMRVRRRLSLFLNVRTQVAADVVTALAEEMDFEYLEIRQ |
| | Full length | LETQADPTGRLLDAWQGRPGASVGRLLELLTKLGRDDVLLELGPSIEEDCQKYILKQQQEEAEK |
| | | PLQVAAVDSSVPRTAELAGITTLDDPLGHMPERFDAFICYCPSDIQFVQEMIRQLEQTNYRLKLC |
| | | VSDRDVLPGTCVWSIASELIEKRCRRMVVVVSDDYLQSKECDFQTKFALSLSPGAHQKRLIPIKY |
| | | KAMKKEFPSILRFITVCDYTNPCTKSWFWTRLAKALSLP |
| 49 | All VL | DIQLTQSPSTLSASMGDRVTITCSASSSVRFIHWYQQKPGKAPKRLIYDTSKLASGVPSRFSGS |
| | (anti-PSCA) | GSGTDFTLTISSLQPEDFATYYCQQWGSSPFTFGQGTKVEIK |
| 51 | All VH | EVQLVEYGGGLVQPGGSLRLSCAASGFNIKDYYIHWVRQAPGKGLEWVAWIDPENGDTEFVPK |
| | (anti-PSCA) | FQGRATMSADTSKNTAYLQMNSLRAEDTAVYYCKTGGFWGQGTLVTVSS |
| 53 | bm2B3 VL | DIQLTQSPSSLSASVGDRVTITCSASSSVRFIHWYQQKPGKAPKRLIYDTSKLASGVPSRFSGSG |
| | (anti-PSCA) | SGTSYTLTISSLQPEDFATYYCQQWSSSPFTFGQGTKVEIK |
| 55 | bm2B3 VH | EVQLVESGGGLVQPGGSLRLSCAASGFNIKDYYIHWVRQAPGKGLEWIGWIDPENGDTEFVPK |
| | (anti-PSCA) | FQGKATMSADTSKNTAYLQMNSLRAEDTAVYYCKTGGFWGQGTLVTVSS |
| 57 | HA1-4.121 | GLQCVAAPRWVLSQVQLQQWGAGLLKPSETLSLTCAVYGGSFSGNYWSWIRQPPGKGLEW |
| | VH | IGEINHSGSTNYNPSLKSRVTISVDTSKNQFSLKLSSVTAADTAVYYCARGGSYNYFDYW |
| | (anti-PSCA) | GQGTLVTVSSASTKGPSVK |
| 59 | HLA1-4.121 | MRLPAQLLGLLMLWVSGSSGDIVMTQSPLSLPVTPGEPASISCRSSQSLLHSNGYNYLVW |
| | VL | YLQKPGQSPQLLIYLGSIRASGVPDRFSGSGSGTDFTLKISRVEAEDVGVYYCMQPLQTP |
| | (anti-PSCA) | ITFGQGTRLEIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVTKSFNRGEC |
| 61 | H1-1.10 VH | QLQESGPGLVKPSQTLSLTCTVSGGSISSGGYYWSWIRQHPGKGLEWIGYIYYSGSTYYN |
| | (anti-PSCA) | PSLKSRVTISVDTSKNQFSLKLSSGTAADTAVYYCARDHITMVRGVPKGMDVWGQGTTVT |
| | | VSSASTKGPSVFPLAPSSKSTSGGTAALG |
| 63 | H1-1.10 VL | QLTQSPSSLSASVGDRVTITCRASQSISRHLNWYQQKPGKAPKFLIYVASSLQSGVPSRF |
| | (anti-PSCA) | SGSGSGTDFTLTISSLQPEDFATYFCQQSYSIPRTFGQGTKVEIKRTVAAPSVFIFPPSD |
| | | EQLKSGTASVVCLLNNFYPREAKVQWK |
| 65 | 1G8 (2B3) | DIQLTQSPSSLSASVGDRVTITCSASSSVRFIHWYQQKPGKAPKRLIYDTSKLASGVPSR |
| | (anti-PSCA | FSGSGSGTDFTLTISSLQPEDFATYYCQQWSSSPFTFGQGTKVEIKGSTSGGGSGGGSGG |
| | scFv) | GGSSEVQLVESGGGLVQPGGSLRLSCAASGFNIKDYYIHWVRQAPGKGLEWVAWIDPENG |
| | | DTEFVPKFQGRATISADTSKNTAYLQMNSLRAEDTAVYYCKTGGFWGQGTLVTVSSAAG |
| 67 | 1G8 (2B3- | DIQLIQSPSSLSASVGDRVTITCSASSSVRFIHWYQQKPGKAPKRLIYDTSKLASGVPSR |
| | C5) | FSGSGSGTDFTLTISSLQPEDFATYYCQQWSSSPFTFGQGTKVEIKGSTSGGGSGGGSGG |
| | (anti-PSCA | GGSSEVQLVESGGGLVQPGGSLRLSCAASGFNIKDYYIHWVRQAPGKGLEWVAWIDPENG |
| | scFv) | DTEFVPKFQGRATISADTSKNTVYLQMNSLRAKDTAVYYCKTGGFWGQGTLVTVSSAAG |
| 69 | 1G8 (2B3-A2) | DIQLTQSPSSLSASVGDRVTITCSASSSVRFIHWYQQKPGKAPKRLIYDTSKLASGVPSR |
| | (anti-PSCA | FSGSGSGTDFTLTISSLQPEDFATYYCQQWGSSPFTFGQGTKVEIKGSTSGGGSGGGSGG |
| | scFv) | GGSEVQLVESGGGLVQPGGSLRLSCAASGFNIKDYYIHWVRQAPGKGLEWVAWIDPEYGD |
| | | SEFVPKFQGRATMSADTSKNTAYLQMNSLRAEDTAVYYCKTGGFWGRGTLVTVSSAAG |
| 71 | 1G8 (2B3- | DIQLTQSPSTLSASMGDRVTITCSASSSVRFIHWYQQKPGKAPKRLIYDTSKLASGVPSR |
| | A11) | FSGSGSGTDFTLTISSLQPEDFATYYCQQWGSSPFTFGQGTKVEIKGSTSGGGSGGGSGG |
| | (anti-PSCA | GGSSEVQLVEYGGGLVQPGGSLRLSCAASGFNIKDYYIHWVRQAPGKGLEWVAWIDPENG |
| | scFv) | DTEFVPKFQGRATMSADTSKNTAYLQMNSLRAEDTAVYYCKTGGFWGQGTLVTVSSAAG |

TABLE 2
nucleic acid sequences

| SEQ ID | ENCODED | |
|---|---|---|
| NO: | PROTEIN | NUCLEIC ACID SEQUENCE |
| 2 | Fv | ATGGGAGTGCAGGTGGAGACTATTAGCCCCGGAGATGGCAGAACATTCCCCAAAAGAGGAC |
| | Human | AGACTTGCGTCGTGCATTATACTGGAATGCTGGAAGACGGCAAGAAGGTGGACAGCAGCCG |
| | FKBP12v36 | GGACCGAAACAAGCCCTTCAAGTTCATGCTGGGGAAGCAGGAAGTGATCCGGGGCTGGGA |
| | | GGAAGGAGTCGCACAGATGTCAGTGGGACAGAGGGCCAAACTGACTATTAGCCCAGACTAC |
| | | GCTTATGGAGCAACCGGCCACCCCGGGATCATTCCCCCTCATGCTACACTGGTCTTCGATG |
| | | TGGAGCTGCTGAAGCTGGAA |
| 4 | 8-amino acid | AGCGGAGGAGGATCCGGAGTGGAC |
| | linker | |
| 6 | T2A | GAAGGCCGAGGGAGCCTGCTGACATGTGGCGATGTGGAGGAAAACCCAGGACCA |
| | polypeptide | |
| 8 | Signal | ATGGAGTTTGGACTTTCTTGGTTGTTTTTGGTGGCAATTCTGAAGGGTGTCCAGTGTAGCAG |
| | peptide | G |
| 10 | Glycine- | GGCGGAGGAAGCGGAGGTGGGGGC |
| | serine linker | |
| 12 | Human CD34 | GAACTTCCTACTCAGGGGACTTTCTCAAACGTTAGCACAAACGTAAGT |
| | epitope | |
| 14 | Human CD8 | CCCGCCCCAAGACCCCCCACACCTGCGCCGACCATTGCTTCTCAACCCCTGAGTTTGAGAC |
| | alpha stalk | CCGAGGCCTGCCGGCCAGCTGCCGGCGGGGCCGTGCATACAAGAGGACTCGATTTCGCTT |
| | | GCGAC |
| 16 | Human CD8 | ATCTATATCTGGGCACCTCTCGCTGGCACCTGTGGAGTCCTTCTGCTCAGCCTGGTTATTAC |
| | trans- | TCTGTACTGTAATCACCGGAATCGCCGCCGCGTTTGTAAGTGTCCCAGG |
| | membrane | |
| | region | |
| | between | |
| | FKBP12-1 | |
| | and FKBP12-2 | |
| 18 | Portion of | AGAGTGAAGTTCAGCAGGAGCGCAGACGCCCCCGCGTACCAGCAGGGCCAGAACCAGCTC |
| | human CD3ζ | TATAACGAGCTCAATCTAGGACGAAGAGAGGAGTACGATGTTTTGGACAAGAGACGTGGCC |
| | in vector | GGGACCCTGAGATGGGGGGAAAGCCGAGAAGGAAGAACCCTCAGGAAGGCCTGTACAATG |
| | | AACTGCAGAAAGATAAGATGGCGGAGGCCTACAGTGAGATTGGGATGAAAGGCGAGCGCC |
| | | GGAGGGGCAAGGGGCACGATGGCCTTTACCAGGGTCTCAGTACAGCCACCAAGGACACCT |
| | | ACGACGCCCTTCACATGCAAGCTCTTCCACCTCGT |
| 20 | P2A | GCAACGAATTTTTCCCTGCTGAAACAGGCAGGGGACGTAGAGGAAAATCCTGGTCCT |
| | polypeptide | |
| 22 | truncated | atggctgcaggaggtcccggcgcggggtctgcggccccggtctcctccacatcctccctt |
| | MyD88 in | cccctggctgctctcaacatgcgagtgcggcgccgcctgtctctgttcttgaacgtgcgg |
| | vector | acacaggtggcggccgactggaccgcgctggcggaggagatggactttgagtacttggag |
| | | atccggcaactggagacacaagcggaccccactggcaggctgctggacgcctggcaggga |
| | | cgccctggcgcctctgtaggccgactgctcgatctgcttaccaagctgggccgcgacgac |
| | | gtgctgctggagctgggacccagcattgaggaggattgccaaaagtatatcttgaagcag |
| | | cagcaggaggaggctgagaagcctttacaggtggccgctgtagacagcagtgtcccacgg |
| | | acagcagagctggcgggcatcaccacacttgatgaccccctggggcatatgcctgagcgt |
| | | ttcgatgccttcatctgctattgccccagcgacatc |
| 24 | Portion of | aaaaaggtggccaagaagccaaccaataaggccccccaccccaagcaggagccccaggag |
| | human CD40 | atcaattttcccgacgatcttcctggctccaacactgctgctccagtgcaggagacttta |
| | in vector | catggatgccaaccggtcacccaggaggatggcaaagagagtcgcatctcagtgcaggag |
| | | agacag |
| 26 | MLEMLE | ATGCTCGAGATGCTGGAG |
| | linker | |
| | encoded 5′ of | |
| | FKBP12v36 | |
| | in vector | |
| 28 | Human | ATGGGAGTGCAGGTGGAAACCATCTCCCCAGGAGACGGGCGCACCTTCCCCAAGCGCGGC |
| | FKBP12 | CAGACCTGCGTGGTGCACTACACCGGGATGCTTGAAGATGGAAAGAAATTTGATTCCTCCC |
| | (Genbank no | GGGACAGAAACAAGCCCTTTAAGTTTATGCTAGGCAAGCAGGAGGTGATCCGAGGCTGGGA |
| | AH002818) | AGAAGGGGTTGCCCAGATGAGTGTGGGTCAGAGAGCCAAACTGACTATATCTCCAGATTAT |
| | | GCCTATGGTGCCACTGGGCACCCAGGCATCATCCCACCACATGCCACTCTCGTCTTCGATG |
| | | TGGAGCTTCTAAAACTGGAATGA |
| 30 | Huma | ATGGCTGCAGGAGGTCCCGGCGCGGGGTCTGCGGCCCCGGTCTCCTCCACATCCTCCCTT |
| | MyD88- | CCCCTGGCTGCTCTCAACATGCGAGTGCGGCGCCGCCTGTCTCTGTTCTTGAACGTGCGGA |
| | encoding | CACAGGTGGCGGCCGACTGGACCGCGCTGGCGGAGGAGATGGACTTTGAGTACTTGGAGA |
| | DNA | TCCGGCAACTGGAGACACAAGCGGACCCCACTGGCAGGCTGCTGGACGCCTGGCAGGGAC |
| | (Genbank no. | GCCCTGGCGCCTCTGTAGGCCGACTGCTCGAGCTGCTTACCAAGCTGGGCCGCGACGACG |
| | U84408) | TGCTGCTGGAGCTGGGACCCAGCATTGAGGAGGATTGCCAAAAGTATATCTTGAAGCAGCA |
| | | GCAGGAGGAGGCTGAGAAGCCTTTACAGGTGGCCGCTGTAGACAGCAGTGTCCCACGGAC |
| | | AGCAGAGCTGGCGGGCATCACCACACTTGATGACCCCCTGGGGCATATGCCTGAGCGTTTC |
| | | GATGCCTTCATCTGCTATTGCCCCAGCGACATCCAGTTTGTGCAGGAGATGATCCGGCAACT |
| | | GGAACAGACAAACTATCGACTGAAGTTGTGTGTGTCTGACCGCGATGTCCTGCCTGGCACC |
| | | TGTGTCTGGTCTATTGCTAGTGAGCTCATCGAAAAGAGGTGCCGCCGGATGGTGGTGGTTG |
| | | TCTCTGATGATTACCTGCAGAGCAAGGAATGTGACTTCCAGACCAAATTTGCACTCAGCCTC |
| | | TCTCCAGGTGCCCATCAGAAGCGACTGATCCCCATCAAGTACAAGGCAATGAAGAAAGAGTT |
| | | CCCCAGCATCCTGAGGTTCATCACTGTCTGCGACTACACCAACCCCTGCACCAAATCTTGGT |
| | | TCTGGACTCGCCTTGCCAAGGCCTTGTCCCTGCCCTGA |
| 32 | Human CD40 | ATGGTTCGTCTGCCTCTGCAGTGCGTCCTCTGGGGCTGCTTGCTGACCGCTGTCCATCCAG |
| | (Genbank no. | AACCACCCACTGCATGCAGAGAAAAACAGTACCTAATAAACAGTCAGTGCTGTTCTTTGTGC |
| | BC012419) | CAGCCAGGACAGAAACTGGTGAGTGACTGCACAGAGTTCACTGAAACGGAATGCCTTCCTT |
| | | GCGGTGAAAGCGAATTCCTAGACACCTGGAACAGAGAGACACACTGCCACCAGCACAAATA |
| | | CTGCGACCCCAACCTAGGGCTTCGGGTCCAGCAGAAGGGCACCTCAGAAACAGACACCATC |
| | | TGCACCTGTGAAGAAGGCTGGCACTGTACGAGTGAGGCCTGTGAGAGCTGTGTCCTGCACC |
| | | GCTCATGCTCGCCCGGCTTTGGGGTCAAGCAGATTGCTACAGGGGTTTCTGATACCATCTG |
| | | CGAGCCCTGCCCAGTCGGCTTCTTCTCCAATGTGTCATCTGCTTTCGAAAAATGTCACCCTT |
| | | GGACAAGCTGTGAGACCAAAGACCTGGTTGTGCAACAGGCAGGCACAAACAAGACTGATGT |
| | | TGTCTGTGGTCCCCAGGATCGGCTGAGAGCCCTGGTGGTGATCCCCATCATCTTCGGGATC |
| | | CTGTTTGCCATCCTCTTGGTGCTGGTCTTTATCAAAAAGGTGGCCAAGAAGCCAACCAATAA |
| | | GGCCCCCCACCCCAAGCAGGAACCCCAGGAGATCAATTTTCCCGACGATCTTCCTGGCTCC |
| | | AACACTGCTGCTCCAGTGCAGGAGACTTTACATGGATGCCAACCGGTCACCCAGGAGGATG |
| | | GCAAAGAGAGTCGCATCTCAGTGCAGGAGAGACAGTGA |
| 34 | Human CD3ζ | ATGAAGTGGAAGGCGCTTTTCACCGCGGCCATCCTGCAGGCACAGTTGCCGATTACAGCCT |
| | (GenBank no. | CCAGCCTCCCCCACCCAACTCAGCAGAGCCCTGAGAAGAAAGTCCTGGGTCCCGGAGGCT |
| | XM_0170028 | GCACCTGCAGACACAACAGATTCTGCAATGAGGCACAGAGCTTTGGCCTGCTGGATCCCAA |
| | 01) | ACTCTGCTACCTGCTGGATGGAATCCTCTTCATCTATGGTGTCATTCTCACTGCCTTGTTCC |
| | | TGAGAGTGAAGTTCAGCAGGAGCGCAGACGCCCCCGCGTACCAGCAGGGCCAGAACCAGCT |
| | | CTATAACGAGCTCAATCTAGGACGAAGAGAGGAGTACGATGTTTTGGACAAGAGACGTGGC |
| | | CGGGACCCTGAGATGGGGGGAAAGCCGAGAAGGAAGAACCCTCAGGAAGGCCTGTACAAT |
| | | GAACTGCAGAAAGATAAGATGGCGGAGGCCTACAGTGAGATTGGGATGAAAGGCGAGCGC |
| | | CGGAGGGGCAAGGGGCACGATGGCCTTTACCAGGGTCTCAGTACAGCCACCAAGGACACC |
| | | TACGACGCCCTTCACATGCAGGCCCTGCCCCCTCGCTAA |
| 36 | MLE linker | ATGCTCGAG |
| 38 | Linker | GGCGGTGGAGGCTCCGGTGGAGGCGGCTCTGGAGGAGGAGGTTCA |
| 40 | ΔCD19 | ATGCCCCCTCCTAGACTGCTGTTTTTCCTGCTCTTTCTCACCCCAATGGAAGTTAGACCTGA |
| | marker | GGAACCACTGGTCGTTAAAGTGGAAGAAGGTGATAATGCTGTCCTCCAATGCCTTAAAGGGA |
| | polypeptide | CCAGCGACGGACCAACGCAGCAACTGACTTGGAGCCGGGAGTCCCCTCTCAAGCCGTTTCT |
| | | CAAGCTGTCACTTGGCCTGCCAGGTCTTGGTATTCACATGCGCCCCCTTGCCATTTGGCTCT |
| | | TCATATTCAATGTGTCTCAACAAATGGGTGGATTCTACCTTTGCCAGCCCGGCCCCCCTTCT |
| | | GAGAAAGCTTGGCAGCCTGGATGGACCGTCAATGTTGAAGGCTCCGGTGAGCTGTTTAGAT |
| | | GGAATGTGAGCGACCTTGGCGGACTCGGTTGCGGACTGAAAAATAGGAGCTCTGAAGGACC |
| | | CTCTTCTCCCTCCGGTAAGTTGATGTCACCTAAGCTGTACGTGTGGGCCAAGGACCGCCCC |
| | | GAAATCTGGGAGGGCGAGCCTCCATGCCTGCCGCCTCGCGATTCACTGAACCAGTCTCTGT |
| | | CCCAGGATCTCACTATGGCGCCCGGATCTACTCTTTGGCTGTCTTGCGGCGTTCCCCCAGA |
| | | TAGCGTGTCAAGAGGACCTCTGAGCTGGACCCACGTACACCCTAAGGGCCCTAAGAGCTTG |
| | | TTGAGCCTGGAACTGAAGGACGACAGACCCGCACGCGATATGTGGGTAATGGAGACCGGC |
| | | CTTCTGCTCCCTCGCGCTACCGCACAGGATGCAGGGAAATACTACTGTCATAGAGGGAATC |
| | | TGACTATGAGCTTTCATCTCGAAATTACAGCACGGCCCGTTCTTTGGCATTGGCTCCTCCGG |
| | | ACTGGAGGCTGGAAGGTGTCTGCCGTAACACTCGCTTACTTGATTTTTTGCCTGTGTAGCCT |
| | | GGTTGGGATCCTGCATCTTCAGCGAGCCCTTGTATTGCGCCGAAAAAGAAAACGAATGACT |
| | | GACCCTACACGACGATTCTGA |
| 42 | Fv | ggagtgcaggtggagactatctccccaggagacgggcgcaccttccccaagcgcggccagac |
| | | ctgcgtggtgcactacaccgggatgcttgaagatggaaagaaagttgattcctcccgggaca |
| | | gaaacaagccctttaagtttatgctaggcaagcaggaggtgatccgaggctgggaagaaggg |
| | | gttgcccagatgagtgtgggtcagagagccaaactgactatatctccagattatgcctatgg |
| | | tgccactgggcacccaggcatcatcccaccacatgccactctcgtcttcgatgtggagcttc |
| | | taaaactggaa |
| 44 | Fv′ | GGcGTcCAaGTcGAaACcATtagtCCcGGcGAtGGcaGaACaTTtCCtAAaaGgGGaCAaACa |
| | | TGtGTcGTcCAtTAtACaGGcATGtTgGAgGAcGGcAAaAAgGTgGAcagtagtaGaGAtcGc |
| | | AAtAAaCCtTTcAAaTTcATGtTgGGaAAaCAaGAaGTcATtaGgGGaTGGGAgGAgGGcGTg |
| | | GCtCAaATGtccGTcGGcCAacGcGCtAAgCTcACcATcagcCCcGAcTAcGCaTAcGGcGCt |
| | | ACcGGaCAtCCcGGaATtATtCCcCCtCAcGCtACctTgGTgTTtGAcGTcGAaCTgtTgAAg |
| | | CTc |
| 46 | FKBP12 | GGcGTGCAaGTGGAaACTATaAGCCCgGGAGAcGGCcGcACATTtCCCAAgAGAGGcCAGACcT |
| | Wild type | GCGTgGTGCAcTATACaGGAATGCTGGAgGACGGgAAGAAaTTCGAtAGCtcCCGGGAtCGAAAt |
| | | AAGCCtTTCAAaTTCATGCTGGGcAAGCAaGAAGTcATCaGaGGCTGGGAaGAAGGcGTCGCc |
| | | CAGATGTCcGTGGGtCAGcGcGCCAAgCTGACaATTAGtCCAGAtTACGCcTATGGcGCAACaG |
| | | GCCAtCCCGGcATCATcCCCCCaCATGCcACACTcGTCTTtGATGTcGAGCTcCTGAAaCTGGAg |
| 48 | MyD88 | atggctgcaggaggtcccggcgcggggtctgcggccccggtctcctccacatcctcccttccc |
| | Full length | ctggctgctctcaacatgcgagtgcggcgccgcctgtctctgttcttgaacgtgcggacacag |
| | | gtggcggccgactggaccgcgctggcggaggagatggactttgagtacttggagatccggcaa |
| | | ctggagacacaagcggaccccactggcaggctgctggacgcctggcagggacgccctggcgcc |
| | | tctgtaggccgactgctcgagctgcttaccaagctgggccgcgacgacgtgctgctggagctg |
| | | ggacccagcattgaggaggattgccaaaagtatatcttgaagcagcagcaggaggaggctgag |
| | | aagcctttacaggtggccgctgtagacagcagtgtcccacggacagcagagctggcgggcatc |
| | | accacacttgatgaccccctggggcatatgcctgagcgtttcgatgccttcatctgctattgc |
| | | cccagcgacatccagtttgtgcaggagatgatccggcaactggaacagacaaactatcgactg |
| | | aagttgtgtgtgtctgaccgcgatgtcctgcctggcacctgtgtctggtctattgctagtgag |
| | | ctcatcgaaaagaggtgccgccggatggtggtggttgtctctgatgattacctgcagagcaag |
| | | gaatgtgacttccagaccaaatttgcactcagcctctctccaggtgcccatcagaagcgactg |
| | | atccccatcaagtacaaggcaatgaagaaagagttccccagcatcctgaggttcatcactgtc |
| | | tgcgactacaccaacccctgcaccaaatcttggttctggactcgccttgccaaggccttgtcc |
| | | ctgccc |
| 50 | A11 VL | GACATCCAACTGACGCAAAGCCCATCTACACTCAGCGCTAGCATGGGGGACAGGGTCACAA |
| | (anti-PSCA) | TCACGTGCTCTGCCTCAAGTTCCGTTAGGTTTATCCATTGGTATCAGCAGAAACCTGGAAAG |
| | | GCCCCAAAAAGACTGATCTATGATACCAGCAAGCTGGCTTCCGGAGTGCCCTCAAGGTTCT |
| | | CAGGATCTGGCAGTGGGACCGATTTCACCCTGACAATTAGCAGCCTTCAGCCAGAGGATTT |
| | | CGCAACCTATTACTGTCAGCAATGGGGGTCCAGCCCATTCACTTTCGGCCAAGGAACAAAG |
| | | GTGGAGATAAAA |
| 52 | A11 VH | GAGGTGCAGCTCGTGGAGTATGGCGGGGGCCTGGTGCAGCCTGGGGGTAGTCTGAGGCTC |
| | (anti-PSCA) | TCCTGCGCTGCCTCTGGCTTTAACATTAAAGACTACTACATACATTGGGTGCGGCAGGCCCC |
| | | AGGCAAAGGGCTCGAATGGGTGGCCTGGATTGACCCTGAGAATGGTGACACTGAGTTTGTC |
| | | CCCAAGTTTCAGGGCAGAGCCACCATGAGCGCTGACACAAGCAAAAACACTGCTTATCTCC |
| | | AAATGAATAGCCTGCGAGCTGAAGATACAGCAGTCTATTACTGCAAGACGGGAGGATTCTG |
| | | GGGCCAGGGAACTCTGGTGACAGTTAGTTCC |
| 54 | bm2B3 VL | GACATCCAGCTGACACAAAGTCCCAGTAGCCTGTCAGCCAGTGTCGGCGATAGGGTGACAA |
| | (anti-PSCA) | TTACATGCTCCGCAAGTAGTAGCGTCAGATTCATACACTGGTACCAGCAGAAGCCTGGGAA |
| | | GGCCCCAAAGAGGCTTATCTACGATACCAGTAAACTCGCCTCTGGAGTTCCTAGCCGGTTTT |
| | | CTGGATCTGGCAGCGGAACTAGCTACACCCTCACAATCTCCAGTCTGCAACCAGAGGACTTT |
| | | GCAACCTACTACTGCCAGCAATGGAGCAGCTCCCCTTTCACCTTTGGGCAGGGTACTAAGG |
| | | TGGAGATCAAG |
| 56 | bm2B3 VH | GAGGTGCAGCTTGTAGAGAGCGGGGGAGGCCTCGTACAGCCAGGGGGCTCTCTGCGCCTG |
| | (anti-PSCA) | TCATGTGCAGCTTCAGGATTCAATATAAAGGACTATTACATTCACTGGGTACGGCAAGCTCC |
| | | CGGTAAGGGCCTGGAATGGATCGGTTGGATCGACCCTGAAAACGGAGATACAGAATTTGTG |
| | | CCCAAGTTCCAGGGAAAGGCTACCATGTCTGCCGATACTTCTAAGAATACAGCATACCTTCA |
| | | GATGAATTCTCTCCGCGCCGAGGACACAGCCGTGTATTATTGTAAAACGGGAGGGTTCTGG |
| | | GGTCAGGGTACCCTTGTGACTGTGTCTTCC |
| 58 | HA1-4.121 | ggtctgcagt gtgtggcagc acccagatgg gtcctgtccc aggtgcagct |
| | VH (anti- | acagcagtgg ggcgcaggac tgttgaagcc ttcggagacc ctgtccctca |
| | PSCA) | cctgcgctgt ctatggtggg tccttcagtg gtaactactg gagctggatc |
| | | cgccagcccc cagggaaggg gctggagtgg attggggaaa tcaatcatag |
| | | tggaagcacc aactacaacc cgtccctcaa gagtcgagtc accatatcag |
| | | tagacacgtc caagaaccag ttctccctga agctgagctc tgtgaccgcc |
| | | gcggacacgg ctgtgtatta ctgtgcgaga ggggggagct acaactactt |
| | | tgactactgg ggccagggaa ccctggtcac cgtctcctca gcctccacca |
| | | agggcccatc ggtcaag |
| 60 | HLA1-4.121 | aaagatcagg actcctcagt tcaccttctc acaatgaggc tccctgctca |
| | VL (anti- | gctcctgggg ctgctaatgc tctgggtctc tggatccagt ggggatattg |
| | PSCA) | tgatgactca gtctccactc tccctgcccg tcacccctgg agagccggcc |
| | | tccatctcct gcaggtctag tcagagcctc ctacatagta atggatacaa |
| | | ctatttggtt tggtacctgc agaagccagg acagtctcca cagctcctga |
| | | tctatttggg ttctattcgg gcctccgggg tccctgacag gttcagtggc |
| | | agtggatcag gcacagattt tacactgaaa atcagcagag tggaggctga |
| | | ggatgttggg gtttattact gcatgcaacc tctacaaact ccgatcacct |
| | | tcggccaagg gacacgactg gagattaaac gaactgtggc tgcaccatct |
| | | gtcttcatct tcccgccatc tgatgagcag ttgaaatctg gaactgcctc |
| | | tgttgtgtgc ctgctgaata acttctatcc cagagaggcc aaagtacagt |
| | | ggaaggtgga taacgccctc caatcgggta actcccagga gagtgtcaca |
| | | gagcaggaca gcaaggacag cacctacagc ctcagcagca ccctgacgct |
| | | gagcaaagca gactacgaga aacacaaagt ctacgcctgc gaagtcaccc |
| | | atcagggcct gagctcgccc gtcacaaaga gcttcaacag gggagagtgt |
| | | tagagggaga agtgccccca cctgctcctc agttccagcc tgaccccctc |
| | | ccatcctttg gcctctgacc ctttttccac aggggaccta cccctattgc |
| | | ggtcctccag ctcatctttc acctcacccc cctcctcctc cttggcttta |
| | | attatgctaa tgttggagga gaatgaataa ataaagtgaa tctttgcaaa |
| | | aaaaaaaaaa aaaaaaaa |
| 62 | H1-1.10 VH | cagctgcagg agtcgggccc aggactggtg aagccttcac agaccctgtc |
| | (anti-PSCA) | cctcacctgc actgtctctg gtggctccat cagcagtggt ggttactact |
| | | ggagctggat ccgccagcac ccagggaagg gcctggagtg gattgggtac |
| | | atctattaca gtgggagcac ctactacaac ccgtccctca agagtcgagt |
| | | taccatatca gtagacacgt ctaagaacca gttctccctg aagctgagct |
| | | ctgggactgc cgcggacacg gccgtgtatt actgtgcgag agaccacatt |
| | | actatggttc ggggagtccc caagggcatg gacgtctggg gccaagggac |
| | | cacggtcacc gtctcctcag cctccaccaa gggcccatcg gtcttccccc |
| | | tggcaccctc ctccaagagc acctctgggg gcacagcggc cctgggc |
| 64 | H1-1.10 VL | cagctgactc agtctccatc ctccctgtct gcatctgtag gagacagagt |
| | (anti-PSCA) | caccatcact tgccgggcaa gtcagagcat tagcaggcat ttaaattggt |
| | | atcagcagaa accagggaaa gcccctaagt tcctgatcta tgttgcatcc |
| | | agtttgcaaa gtggggtccc atcaagattc agtggcagtg gatctgggac |
| | | agatttcact ctcaccatca gcagtctgca acctgaagat tttgcaactt |
| | | acttctgtca acagagttac agtatccccc ggacgttcgg ccaagggacc |
| | | aaggtggaaa tcaaacgaac tgtggctgca ccatctgtct tcatcttccc |
| | | gccatctgat gagcagttga aatctggaac tgcctctgtt gtgtgcctgc |
| | | tgaataactt ctatcccaga gaggccaaag tacagtggaa g |

### Example 3: Examples of Embodiments

The examples set forth below illustrate certain embodiments and do not limit the technology.

A1. A heterogeneous T cell population comprising modified and unmodified T cells, wherein the modified T cells comprise a) a first polynucleotide encoding an inducible MyD88/CD40 polypeptide and b) a second polynucleotide encoding a prostate stem cell antigen chimeric antigen receptor (PSCA-CAR) polypeptide.

A2. The T cell population of embodiment A1, wherein about 20% to about 90% of the T cells are modified T cells.

A3. The T cell population of embodiment A1, wherein about 20% to about 80% of the T cells are modified T cells.

A4. The T cell population of embodiment A1, wherein about 20% to about 70% of the T cells are modified T cells.

A5. The T cell population of embodiment A1, wherein about 20% to about 60% of the T cells are modified T cells.

A6. The T cell population of embodiment A1, wherein about 20% to about 50% of the T cells are modified T cells.

A7. The T cell population of any one of embodiments A1 to A6, suspended in freezing medium.

A8. A cryopreserved T cell population of any one of embodiments A1 to A6.

A9. Reserved

A10. The T cell population of any one of embodiments A1 to A9, comprising about 20×10⁶ to 120×10⁶ cells per milliliter in a diluent suitable for infusion into a subject.

A11. The T cell population of any one of embodiments A1 to A10, wherein the inducible MyD88/CD40 polypeptide comprises
i) two multimeric ligand binding regions;
ii) a MyD88 polypeptide or a truncated MyD88 polypeptide lacking a TIR domain; and
iii) a CD40 cytoplasmic polypeptide region lacking a CD40 extracellular domain.

A12. The T cell population of embodiment A11, wherein each multimeric ligand binding region comprises an FKBP12 variant polypeptide.

A13. The T cell population of embodiment A12, wherein each FKBP12 variant polypeptide binds with higher affinity to the multimeric ligand than the wild type FKBP12 polypeptide.

A14. The T cell population of any one of embodiments A12 or A13, wherein each FKBP12 variant polypeptide comprises an amino acid substitution at position 36 that binds with higher affinity to the multimeric ligand than the wild type FKBP12 polypeptide.

A15. The T cell population of embodiment A14, wherein the amino acid substitution at position 36 is selected from the group consisting of valine, isoleucine, leucine, and alanine.

A16. The T cell population of embodiment A15, wherein each multimeric ligand binding region is an FKB12v36 region.

A17. The T cell population of any one of embodiments A1 to A16, wherein the truncated MyD88 polypeptide has the amino acid sequence of SEQ ID NO: 21, or a functional fragment thereof.

A18. The T cell population of any one of embodiments A1 to A17, wherein the truncated MyD88 polypeptide does not comprise contiguous amino acid residues 156 to the C-terminus of the full length MyD88 polypeptide.

A19. The T cell population of any one of embodiments A1 to A17, wherein the truncated MyD88 polypeptide does not comprise contiguous amino acid residues 152 to the C-terminus of the full length MyD88 polypeptide.

A20. The T cell population of any one of embodiments A1 to A17, wherein the truncated MyD88 polypeptide does not comprise contiguous amino acid residues 173 to the C-terminus of the full length MyD88 polypeptide.

A21. The T cell population of any one of embodiments A18 to A20, wherein the full length MyD88 polypeptide comprises the amino acid sequence of SEQ ID NO: 47.

A22. The T cell population of any one of embodiments A1 to A17, wherein the truncated MyD88 polypeptide consists of the amino acid sequence of SEQ ID NO: 21.

A23. The T cell population of any one of embodiments A1 to A22, wherein the cytoplasmic CD40 polypeptide comprises the amino acid sequence of SEQ ID NO: 23, or a functional fragment thereof.

A24. The T cell population of any one of embodiments A1 to A22, wherein the cytoplasmic CD40 polypeptide consists of the amino acid sequence of SEQ ID NO: 23, or a functional fragment thereof.

A25. The T cell population of any one of embodiments A1 to A24, wherein the chimeric antigen receptor comprises an antigen recognition moiety, a transmembrane region, and a T cell activation molecule.

A26. The T cell population of embodiment A25, wherein the antigen recognition moiety is derived from a PSCA antibody selected from the group consisting of 1G8, 2A2, 2H9, 3C5, 3E6, 3G3, 4A10, and A11, wherein 1G8, 2A2, 2H9, 3C5, 3E6, 3G3, and 4A10 are produced by the hybridomas designated HB-12612, HB-12613, HB-12614, HB-12616, HB-12618, HB-12615, and HB-12617, respectively, as deposited with the American Type Culture Collection.

A27. The T cell population of any one of embodiments A25 or A26, wherein the antigen recognition moiety comprises the complementarity determining regions (CDRs) of the heavy chain variable domain and the light chain variable domain of an amino acid sequence selected from the group consisting of SEQ ID NOs: 65, 67, 69, and 71.

A28. The T cell population of embodiment A25, wherein the antigen recognition moiety comprises a variable heavy chain amino acid sequence and a variable light chain amino acid sequence selected from the group consisting of SEQ ID NOs: 51 and 49; SEQ ID NOs: 55 and 53; SEQ ID NOs: 57 and 59; and SEQ ID NOs: 61 and 63.

B1. A method for treating a subject, wherein the subject has been diagnosed with a disease associated with the presence of one or more solid tumors expressing prostate stem cell antigen (PSCA), comprising administering to the subject a pharmacological agent, wherein the subject has previously received an infusion of a T cell population of any one of embodiments A1 to A28.

B2. The method of embodiment B1, wherein a plurality of doses of the pharmacological agent is administered to the subject.

B3. The method of embodiment B1 or B2, wherein the pharmacological agent is administered to the subject once per day.

B4. The method of embodiment B1 or B2, wherein the pharmacological agent is administered to the subject three times per week.

B5. The method of embodiment B1 or B2, wherein the pharmacological agent is administered to the subject twice per week.

B6. The method of embodiment B1 or B2, wherein the pharmacological agent is administered to the subject once per week.

B7. The method of embodiment B1 or B2, wherein the pharmacological agent is administered to the subject once every other week.

B8. The method of embodiment B1 or B2, wherein the pharmacological agent is administered to the subject twice per month.

B9. The method of embodiment B1 or B2, wherein the pharmacological agent is administered to the subject once per month.

B10. The method of any one of embodiments B1 to B9, further comprising monitoring tumor burden in the subject.

B11. The method of any one of embodiments B1 to B9, further comprising monitoring PSCA copy number in the subject.

B12. The method of any one of embodiments B1 to B11, further comprising monitoring the number or concentration of modified T cells in the subject's peripheral blood.

B13. The method of any one of embodiments B1 to B12, wherein the first dose of the pharmacological composition is administered four or more days following infusion of the T cell population.

B14. The method of any one of embodiments B1 to B12, wherein the first dose of the pharmacological composition is administered seven or more days following infusion of the T cell population.

B15. The method of any one of embodiments B1 to B14, comprising increasing the frequency of administration of the pharmacological agent after determining that the tumor burden or the PSCA copy number in the subject is not sufficiently reduced, or that the disease is progressing.

B16. The method of any one of embodiments B1 to B14, comprising increasing the frequency of administration of the pharmacological agent after determining that the tumor burden or the PSCA copy number in the subject is reduced less than 50%.

B17. The method of any one of embodiments B1 to B14, comprising increasing the frequency of administration of the pharmacological agent after determining that the tumor burden or the PSCA copy number in the subject is reduced less than 20%.

B18. The method of any one of embodiments B10 to B16, comprising reducing the frequency of administration of the pharmacological agent after determining that the tumor burden or the PSCA copy number in the subject is reduced 50 percent or more.

B19. The method of any one of embodiments B10 to B16, comprising reducing the frequency of administration of the pharmacological agent after determining that the tumor burden or the PSCA copy number in the subject is reduced 90 percent or more.

B20. The method of any one of embodiments B10 to B16, comprising reducing the frequency of administration of the pharmacological agent after determining that the subject has reached the status of stable disease or remission.

B21. The method of any one of embodiments B18 to B20, comprising reducing the frequency of administration of the pharmacological agent to one time per month.

B22. The method of any one of embodiments B18 to B20, comprising reducing the frequency of administration of the pharmacological agent to one time every two months.

B23. The method of any one of embodiments B18 to B20, comprising reducing the frequency of administration of the pharmacological agent to one time every four months.

B24. The method of any one of embodiments B18 to B20, comprising reducing the frequency of administration of the pharmacological agent to one time every six months.

B25. The method of any one of embodiments B18 to B20, comprising reducing the frequency of administration of the pharmacological agent to one time per month.

B26. The method of any one of embodiments B18 to B20, comprising reducing the frequency of administration of the pharmacological agent to one time every year.

B28. The method of any one of embodiments B18 to B20, comprising ceasing administration of the pharmacological agent.

B29. The method of any one of embodiments B21 to B26, comprising determining that the subject has a need for increased CAR-T cell activity and increasing the frequency of administration of the pharmacological agent.

B30. The method of any one of embodiments B21 to B26, comprising
i) determining that the tumor burden or the PSCA copy number in the subject is increased compared to the tumor burden or the PSCA copy number in the subject before reducing the frequency of administration of the pharmacological agent; and
ii) resuming a schedule of administration, or increasing the frequency of administration of the pharmacological agent.

B31. The method of any one of embodiments B1 to B30, wherein the pharmacological agent is rimiducid.

B32. The method of embodiment B31, wherein each administration of rimiducid to the subject is about 0.4 mg/kg subject body weight.

C1. The method of any one of embodiments B1 to B32, wherein the subject is not treated with IL-2 following infusion of the T cell population.

C2. The method of any one of embodiments B1 to C1, wherein the subject has been diagnosed with a tumor in the pancreas, prostate, stomach, ovary, or bladder.

C3. The method of any one of embodiments B1 to C1, wherein the subject has been diagnosed with pancreatic, prostate, gastric, ovarian, or bladder cancer.

C4. The method of any one of embodiments B1 to C1, wherein the subject has been diagnosed with hormone refractory prostate cancer, prostate adenocarcinoma, or metastatic prostate cancer.

C5. The method of any one of embodiments B1 to C1, wherein the subject has been diagnosed with gastroesophogeal junction cancer, diffuse gastric cancer, intestinal gastric cancer, or gastric adenocarcinoma.

C6. The method of any one of embodiments B1 to C1, wherein the subject has been diagnosed with pancreatic adenocarcinoma, resectable pancreatic cancer, or borderline resectable pancreatic cancer.

C7. The method of any one of embodiments B1 to C1, wherein a sample obtained from the subject before administration of the pharmacological agent comprises 5,000 or more copies of prostate stem cell antigen (PSCA) per 10⁴ copies of human beta-actin (hACTB).

C8. The method of embodiment C7, wherein the sample comprises 10,000 or more copies of PSCA per 10⁴ copies of hACTB.

C9. The method of embodiment C7, wherein the sample comprises 20,000 or more copies of PSCA per 10⁴ copies of hACTB.

010. The method of embodiment C7, wherein the sample comprises 30,000 or more copies of PSCA per 10⁴ copies of hACTB.

C11. The method of embodiment C7, wherein the sample comprises 100,000 or more copies of PSCA per 10⁴ copies of hACTB.

C12. The method of embodiment C7, wherein the sample comprises 200,000 or more copies of PSCA per 10⁴ copies of hACTB.

C13. The method of embodiment C7, wherein the sample comprises 300,000 or more copies of PSCA per 10⁴ copies of hACTB.

C14. The method of any one of embodiments B1 to C13, wherein following administration of the pharmacological agent, modified T cells persist in the subject for over 1 month.

C15. The method of any one of embodiments B1 to C13, wherein following administration of the pharmacological agent, modified T cells persist in the subject for over 2 months.

C16. The method of any one of embodiments B1 to C13, wherein following administration of the pharmacological agent, modified T cells persist in the subject for over 4 months.

C17. The method of any one of embodiments B1 to C13, wherein following administration of the pharmacological agent, modified T cells persist in the subject for over 6 months.

C18. The method of any one of embodiments B1 to C17, wherein following administration of the pharmacological agent, the modified cells expand 3 or more fold in the peripheral blood of the subject.

C19. The method of any one of embodiments B1 to C17, wherein following administration of the pharmacological agent, the modified cells expand 5 or more fold in the peripheral blood of the subject.

C20. The method of any one of embodiments B1 to C17, wherein following administration of the pharmacological agent, the modified cells expand 10 or more fold in the peripheral blood of the subject.

C21. The method of any one of embodiments B1 to C17, wherein following administration of the pharmacological agent, the modified cells expand 15 or more fold in the peripheral blood of the subject.

C22. The method of any one of embodiments B1 to C17, wherein following administration of the pharmacological agent, the modified cell expand 20 or more fold in the peripheral blood of the subject.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of this technology.

Certain embodiments of the technology are set forth in the claim(s) that follow(s).

What is claimed is:

1. A method for treating a human subject, wherein the subject has been diagnosed with a disease associated with the presence of one or more solid tumors expressing prostate stem cell antigen (PSCA), comprising:
administering to the subject 0.3×10⁶ cells/kg to about 9×10⁶ cells/kg of modified T cells comprising:
(i) a first polynucleotide encoding an inducible MyD88/CD40 polypeptide and
(ii) a second polynucleotide encoding a prostate stem cell antigen chimeric antigen receptor (PSCA-CAR) polypeptide.

2. The method of claim 1 further comprising administering an unmodified polyclonal T cells in an amount between 0.1×10⁶ cells/kg to 30×10⁶ cells/kg.

3. The method of claim 2, wherein the subject is administered an infusion of a heterogeneous T cell population comprising the modified and the unmodified T cells, wherein about 20% to about 90% of the T cells are modified T cells.

4. The method of claim 2, wherein the subject is administered an infusion of a heterogeneous T cell population comprising the modified and the unmodified T cells, wherein about 20% to about 80% of the T cells are modified T cells.

5. The method of claim 2, wherein the subject is administered an infusion of a heterogeneous T cell population comprising the modified and the unmodified T cells, wherein about 20% to about 70% of the T cells are modified T cells.

6. The method of claim 2, wherein the subject is administered an infusion of a heterogeneous T cell population comprising the modified and the unmodified T cells, wherein about 20% to about 60% of the T cells are modified T cells.

7. The method of claim 2, wherein the subject is administered an infusion of a heterogeneous T cell population comprising the modified and the unmodified T cells, wherein about 20% to about 50% of the T cells are modified T cells.

8. The method of any of the claims 1 to 7, wherein the T cells are administered to the subject in a diluent suitable for infusion into the subject comprising about 20×10⁶ to 120×10⁶ cells per milliliter.

9. The method of anyone of claims 1-8, further comprising administering to the subject a plurality of doses of rimiducid.

10. The method of claim 9, wherein rimiducid is administered to the subject once per day.

11. The method of claim 9, wherein rimiducid is administered to the subject three times per week.

12. The method of claim 9, wherein rimiducid is administered to the subject twice per week.

13. The method of claim 9, wherein rimiducid is administered to the subject once per week.

14. The method of claim 9, wherein rimiducid is administered to the subject once every other week.

15. The method of claim 9, wherein rimiducid is administered to the subject twice per month.

16. The method of anyone of claims 9-15, wherein each administration of rimiducid to the subject is about 0.4 mg/kg subject body weight.

17. The method of claim 9, wherein rimiducid is administered to the subject once per week, wherein each administration of rimiducid to the subject is about 0.4 mg/kg subject body weight.

18. The method of claim 9, wherein rimiducid is administered to the subject once per day, wherein each administration of rimiducid to the subject is about 0.4 mg/kg subject body weight.

19. The method of claim 9, wherein rimiducid is administered to the subject three times per week, wherein each administration of rimiducid to the subject is about 0.4 mg/kg subject body weight.

20. The method of claim 9, wherein rimiducid is administered to the subject twice per week, wherein each administration of rimiducid to the subject is about 0.4 mg/kg subject body weight.

21. The method of claim 9, wherein rimiducid is administered to the subject once every other week, wherein each administration of rimiducid to the subject is about 0.4 mg/kg subject body weight.

22. The method of any preceding claim wherein the solid tumor is pancreatic cancer, gastric cancer or prostate cancer.

23. The method of any preceding claim, wherein the inducible MyD88/CD40 polypeptide comprises
i) two multimeric ligand binding regions;
ii) a MyD88 polypeptide or a truncated MyD88 polypeptide lacking a TIR domain; and
iii) a CD40 cytoplasmic polypeptide region lacking a CD40 extracellular domain.

24. The method of claim 23, wherein each multimeric ligand binding region comprises an FKBP12 variant polypeptide.

25. The method of claim 24, wherein each FKBP12 variant polypeptide comprises an amino acid substitution at position 36 that binds with higher affinity to the multimeric ligand than the wild type FKBP12 polypeptide.

26. The method of claim 25, wherein the amino acid substitution at position 36 is selected from the group consisting of valine, isoleucine, leucine, and alanine.

27. The method of claim 26, wherein each multimeric ligand binding region is an FKB12v36 region.

28. The method of any one of claims 23-27, wherein the truncated MyD88 polypeptide has the amino acid sequence of SEQ ID NO: 21, or a functional fragment thereof.

29. The method of any one of claims 23-27, wherein the CD40 cytoplasmic polypeptide comprises the amino acid sequence of SEQ ID NO: 23, or a functional fragment thereof.

30. The method of any preceding claims, wherein the PSCA-CAR polypeptide comprises an antigen recognition moiety, a transmembrane region, and a T cell activation molecule.

31. The method of claim 30, wherein the antigen recognition moiety is derived from a PSCA antibody selected from the group consisting of 1G8, 2A2, 2H9, 3C5, 3E6, 3G3, 4A10, and A11, wherein 1G8, 2A2, 2H9, 3C5, 3E6, 3G3, and 4A10 are produced by the hybridomas designated HB-12612, HB-12613, HB-12614, HB-12616, HB-12618, HB-12615, and HB-12617, respectively, as deposited with the American Type Culture Collection.

32. The method of claim 30, wherein the antigen recognition moiety comprises the complementarity determining regions (CDRs) of the heavy chain variable domain and the light chain variable domain of an amino acid sequence selected from the group consisting of SEQ ID NOs: 65, 67, 69, and 71.

33. The method of claim 30, wherein the antigen recognition moiety comprises a variable heavy chain amino acid sequence and a variable light chain amino acid sequence selected from the group consisting of SEQ ID NOs: 51 and 49; SEQ ID NOs: 55 and 53; SEQ ID NOs: 57 and 59; and SEQ ID NOs: 61 and 63.

34. The method of claim 30, wherein the antigen recognition moiety is a single-chain variable (scFV) comprising a variable light chain amino acid sequence of SEQ ID NO: 49 and a variable heavy chain amino acid sequence of SEQ ID NO: 51.

35. The method of claim 30, wherein the antigen recognition moiety is a scFV comprising a variable light chain amino acid sequence of SEQ ID NO:53 and a variable heavy chain amino acid sequence of SEQ ID NO:55.

36. The method of claim 30, wherein the antigen recognition moiety is a scFV comprising a variable light chain amino acid sequence of SEQ ID NO: 57 and a variable heavy chain amino acid sequence of SEQ ID NO: 59.

37. The method of claim 30, wherein the antigen recognition moiety is a scFV comprising a variable light chain amino acid sequence of SEQ ID NO: 61 and a variable heavy chain amino acid sequence of SEQ ID NO: 63.

38. The method of claim 30, wherein the antigen recognition moiety is a scFV comprising a variable light chain amino acid sequence of SEQ ID NO: 65 and a variable heavy chain amino acid sequence of SEQ ID NO: 67.

39. The method of claim 30, wherein the antigen recognition moiety is a scFV comprising a variable light chain amino acid sequence of SEQ ID NO: 69 and a variable heavy chain amino acid sequence of SEQ ID NO: 71.

40. A heterogeneous T cell population comprising modified and unmodified T cells, wherein the modified T cells comprise a) a first polynucleotide encoding an inducible MyD88/CD40 polypeptide and b) a second polynucleotide encoding a PSCA-CAR polypeptide.

41. The T cell population of claim 40, wherein about 20% to about 90% of the T cells are modified T cells.

42. The T cell population of claim 40, wherein about 20% to about 80% of the T cells are modified T cells.

43. The T cell population of claim 40, wherein about 20% to about 70% of the T cells are modified T cells.

44. The T cell population of claim 40, wherein about 20% to about 60% of the T cells are modified T cells.

45. The T cell population of claim 40, wherein about 20% to about 50% of the T cells are modified T cells.

46. The T cell population of any one of claims 40 to 45, suspended in freezing medium.

47. A cryopreserved T cell population of any one of claims 40 to 45.

48. The T cell population of any one of claims 40 to 47, comprising about 20×10⁶ to 150×10⁶ cells per milliliter in a diluent suitable for infusion into a subject.

49. The T cell population of any one of claims 40 to 47, comprising about 20×10⁶ to 120×10⁶ cells per milliliter in a diluent suitable for infusion into a subject.

50. The T cell population of any one of claims 40 to 49, wherein the inducible MyD88/CD40 polypeptide comprises
i) two multimeric ligand binding regions;
ii) a MyD88 polypeptide or a truncated MyD88 polypeptide lacking a TIR domain; and
iii) a CD40 cytoplasmic polypeptide region lacking a CD40 extracellular domain.

51. The T cell population of claim 50, wherein each multimeric ligand binding region comprises an FKBP12 variant polypeptide.

52. The T cell population of claim 51, wherein each FKBP12 variant polypeptide binds with higher affinity to the multimeric ligand than the wild type FKBP12 polypeptide.

53. The T cell population of claim 51 or 52, wherein each FKBP12 variant polypeptide comprises an amino acid substitution at position 36 that binds with higher affinity to the multimeric ligand than the wild type FKBP12 polypeptide.

54. The T cell population of claim 53, wherein the amino acid substitution at position 36 is selected from the group consisting of valine, isoleucine, leucine, and alanine.

55. The T cell population of claim 54, wherein each multimeric ligand binding region is an FKB12v36 region.

56. The T cell population of any one of claims 40 to 55, wherein the truncated MyD88 polypeptide has the amino acid sequence of SEQ ID NO: 21, or a functional fragment thereof.

57. The T cell population of any one of claims 40 to 56, wherein the truncated MyD88 polypeptide consists of the amino acid sequence of SEQ ID NO: 21.

58. The T cell population of any one of claims 40 to 57, wherein the CD40 cytoplasmic polypeptide comprises the amino acid sequence of SEQ ID NO: 23, or a functional fragment thereof.

59. The T cell population of any one of claims 40 to 57, wherein the CD40 cytoplasmic polypeptide consists of the amino acid sequence of SEQ ID NO: 23, or a functional fragment thereof.

60. The T cell population of any one of claims 40 to 59, wherein the PSCA-CAR polypeptide comprises an antigen recognition moiety, a transmembrane region, and a T cell activation molecule.

61. The T cell population of claim 60, wherein the antigen recognition moiety is derived from a PSCA antibody selected from the group consisting of 1G8, 2A2, 2H9, 3C5, 3E6, 3G3, 4A10, and A11, wherein 1G8, 2A2, 2H9, 3C5, 3E6, 3G3, and 4A10 are produced by the hybridomas designated HB-12612, HB-12613, HB-12614, HB-12616, HB-12618, HB-12615, and HB-12617, respectively, as deposited with the American Type Culture Collection.

62. The T cell population of any one of claim 60 or 61, wherein the antigen recognition moiety comprises the complementarity determining regions (CDRs) of the heavy chain variable domain and the light chain variable domain of an amino acid sequence selected from the group consisting of SEQ ID NOs: 65, 67, 69, and 71.

63. The T cell population of claim 60, wherein the antigen recognition moiety comprises a variable heavy chain amino acid sequence and a variable light chain amino acid sequence selected from the group consisting of SEQ ID NOs: 51 and 49; SEQ ID NOs: 55 and 53; SEQ ID NOs: 57 and 59; and SEQ ID NOs: 61 and 63.

64. The T cell population of claim 60, wherein the antigen recognition moiety is a scFV comprising a variable light chain amino acid sequence of SEQ ID NO: 49 and a variable heavy chain amino acid sequence of SEQ ID NO: 51.

65. The T cell population of claim 60, wherein the antigen recognition moiety is a scFV comprising a variable light chain amino acid sequence of SEQ ID NO:53 and a variable heavy chain amino acid sequence of SEQ ID NO:55.

66. The T cell population of claim 60, wherein the antigen recognition moiety is a scFV comprising a variable light chain amino acid sequence of SEQ ID NO: 57 and a variable heavy chain amino acid sequence of SEQ ID NO: 59.

67. The T cell population of claim 60, wherein the antigen recognition moiety is a scFV comprising a variable light chain amino acid sequence of SEQ ID NO: 61 and a variable heavy chain amino acid sequence of SEQ ID NO: 63.

68. The T cell population of claim 60, wherein the antigen recognition moiety is a scFV comprising a variable light chain amino acid sequence of SEQ ID NO: 65 and a variable heavy chain amino acid sequence of SEQ ID NO: 67.

69. The T cell population of claim 60, wherein the antigen recognition moiety is a scFV comprising a variable light chain amino acid sequence of SEQ ID NO: 69 and a variable heavy chain amino acid sequence of SEQ ID NO: 71.
